(19) United States
(12) Patent Application Publication
(10) Pub. No.: US 2023/0139045 A1
(43) Pub. Date: May 4, 2023

(54) COMPOUND FOR ORGANIC OPTOELECTRONIC DEVICE, COMPOSITION FOR ORGANIC OPTOELECTRONIC DEVICE, ORGANIC OPTOELECTRONIC DEVICE AND DISPLAY DEVICE

(71) Applicant: SAMSUNG SDI CO., LTD., Yongin-si (KR)

(72) Inventors: Wook KIM, Suwon-si (KR); Seonyeong GWAK, Suwon-si (KR); Jaehoon KIM, Suwon-si (KR); Hyung Sun KIM, Suwon-si (KR); Mijin LEE, Suwon-si (KR); Yoonman LEE, Suwon-si (KR); Hanill LEE, Suwon-si (KR); Suheon IM, Suwon-si (KR); Sung-Hyun JUNG, Suwon-si (KR)

(21) Appl. No.: 17/903,190

(22) Filed: Sep. 6, 2022

(30) Foreign Application Priority Data
Sep. 3, 2021 (KR) 10-2021-0117780
Aug. 31, 2022 (KR) 10-2022-0110105

(51) Int. Cl. C07D 403/10; C07D 405/14; C07D 409/14; C07F 7/08; C07D 403/04; C07D 491/048; C07D 495/04; C07D 487/04; H01L 51/00; H01L 51/50

(52) U.S. Cl. CPC C07D 403/10; C07D 403/04; C07D 405/14; C07D 409/14; C07D 487/04; C07D 491/048; C07D 495/04; C07F 7/0816; H01L 51/0067; H01L 51/0071; H01L 51/0072; H01L 51/0073; H01L 51/0074; H01L 51/0091; H01L 51/5012; C07B 2200/05

(57) ABSTRACT

Provided a compound for an organic optoelectronic device represented by Chemical Formula 1, a composition for an organic optoelectronic device including the same, an organic optoelectronic device, and a display device. Details of Chemical Formula 1 are as defined in the specification.

### CROSS-REFERENCE TO RELATED APPLICATION

This application claims priority to and the benefit of Korean Patent Application No. 10-2021-0117780 filed in the Korean Intellectual Property Office on Sep. 3, 2021, and Korean Patent Application No. 10-2022-0110105 filed in the Korean Intellectual Property Office on Aug. 31, 2022, the entire contents of which are incorporated herein by reference.

### BACKGROUND

### 1. Field

Embodiments relate to a compound for an organic optoelectronic device, a composition for an organic optoelectronic device, an organic optoelectronic device, and a display device.

### 2. Description of the Related Art

An organic optoelectronic device (e.g., an organic optoelectronic diode) is a device capable of converting electrical energy to optical energy, optical energy to electrical energy, or electrical energy and optical energy to each other.

An organic optoelectronic device may be classified as follows in accordance with its driving principles. One is a photoelectric device that generates electrical energy by separating excitons formed by light energy into electrons and holes, and transferring the electrons and holes to different electrodes, respectively. Another is a light emitting device that generates light energy from electrical energy by supplying voltage or current to the electrodes.

Examples of the organic optoelectronic device include an organic photoelectric element, an organic light emitting diode, an organic solar cell, and an organic photo conductor drum.

Of these, an organic light emitting diode (OLED) has recently drawn attention due to an increase in demand for flat panel displays. The organic light emitting diode is a device that converts electrical energy into light, and the performance of the organic light emitting diode is greatly influenced by an organic material between electrodes.

### SUMMARY

An embodiment is directed to a compound for an organic optoelectronic device, the compound being represented by Chemical Formula 1:

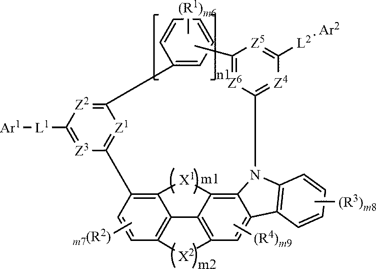

In Chemical Formula 1,
m1 and m2 may each independently be an integer of 0 or 1,
X¹ and X² may each independently be NR^{g}, O, S, CR^{a}R^{b}, or SiR^{c}R^{d},
n1 may be an integer of 1 to 5,
Z¹ to Z⁶ may each independently be N or CR^{e},
at least two of Z¹ to Z³ may be N; at least two of Z⁴ to Z⁶ may be N; or at least two of Z¹ to Z³ may be N and at least two of Z⁴ to Z⁶ may be N,
R^{a}, R^{b}, R^{c}, R^{d} and R^{g} may each independently be a substituted or unsubstituted C1 to C30 alkyl group, or a substituted or unsubstituted C6 to C30 aryl group,
R^{e} and R¹ toR⁴ may each independently be hydrogen, deuterium, a halogen, a substituted or unsubstituted C1 to C30 alkyl group, or a substituted or unsubstituted C6 to C30 aryl group,
m6 and m8 may each independently be an integer of 1 to 4,
R^{g} may be phenyl,
m7 may be an integer of 1 to 4,
m9 may be an integer of 1 to 3,
Ar¹ and Ar² may each independently be a substituted or unsubstituted C6 to C30 aryl group or a substituted or unsubstituted C2 to C30 heterocyclic group, and
L¹ and L² may each independently be a single bond, a substituted or unsubstituted C6 to C30 arylene group, or a substituted or unsubstituted C2 to C30 heterocyclic group.

An embodiment is directed to a composition for an organic optoelectronic device including the first compound and the second compound.

The first compound may be the same as described above, and the second compound may be a compound for an organic optoelectronic device represented by Chemical Formula 2; or a compound for an organic optoelectronic device represented by a combination of Chemical Formula 3 and Chemical Formula 4:

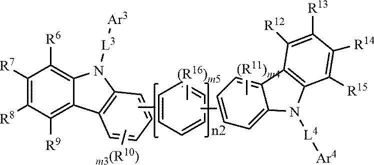

In Chemical Formula 2,
Ar³ and Ar⁴ may each independently be a substituted or unsubstituted C6 to C20 aryl group or a substituted or unsubstituted C2 to C30 heterocyclic group,
L³ and L⁴ may each independently be a single bond or a substituted or unsubstituted C6 to C20 arylene group,
R⁶ to R¹⁶ may each independently be hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
m3 and m4 may each independently be an integer from 1 to 3,
m5 may be an integer from 1 to 4, and
n2 may be an integer from 0 to 2;

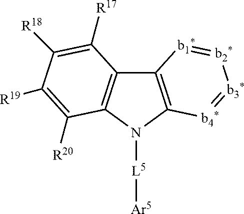

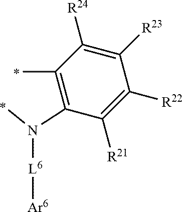

In Chemical Formulas 3 and 4,
Ar⁵ and Ar⁶ may each independently be a substituted or unsubstituted C6 to C20 aryl group or a substituted or unsubstituted C2 to C30 heterocyclic group,
adjacent two of b₁* to b₄* of Chemical Formula 3 may each be a carbon (C) linked to * in Chemical Formula 4,
the other two not linked to * of Chemical Formula 4 of b₁* to b₄* in Chemical Formula 3 may each independently be C- L^{d}-R^{f},
L^{d}, L⁵, and L⁶ may each independently be a single bond or a substituted or unsubstituted C6 to C20 arylene group, and
R^{f} and R¹⁷ to R²⁴ may each independently be hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

An embodiment is directed to an organic optoelectronic device that includes an anode and a cathode facing each other, and at least one organic layer between the anode and the cathode, wherein the organic layer includes the compound for the organic optoelectronic device or the composition for the organic optoelectronic device.

An embodiment is directed to a display device including the organic optoelectronic device.

### BRIEF DESCRIPTION OF THE DRAWING

Features will become apparent to those of skill in the art by describing in detail example embodiments with reference to the attached drawing in which:

The FIGURE is a cross-sectional view illustrating an organic light emitting diode according to an example embodiment.

### DETAILED DESCRIPTION

Example embodiments will now be described more fully hereinafter with reference to the accompanying drawing; however, they may be embodied in different forms and should not be construed as limited to the embodiments set forth herein. Rather, these embodiments are provided so that this disclosure will be thorough and complete, and will fully convey example implementations to those skilled in the art. In the drawing figure, the dimensions of layers and regions may be exaggerated for clarity of illustration. Like reference numerals refer to like elements throughout.

In one example of the present disclosure, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In specific example of the present disclosure, the “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In specific example of the present disclosure, the “substituted” refers to replacement of at least one hydrogen of a substituent or a compound by deuterium, a C1 to C20 alkyl group, a C6 to C30 aryl group, or a cyano group. In specific example of the present disclosure, the “substituted” refers to replacement of at least one hydrogen of a substituent or a compound by deuterium, a C1 to C5 alkyl group, a C6 to C18 aryl group, or a cyano group. In specific example of the present disclosure, the “substituted” refers to replacement of at least one hydrogen of a substituent or a compound by deuterium, a cyano group, a methyl group, an ethyl group, a propyl group, a butyl group, a phenyl group, a biphenyl group, a terphenyl group, or a naphthyl group.

“Unsubstituted” refers to non-replacement of a hydrogen atom by another substituent and remaining of the hydrogen atom.

As used herein, “hydrogen substitution (-H)” may include deuterium substitution (-D) or tritium substitution (-T).

As used herein, when a definition is not otherwise provided, “hetero” refers to one including one to three heteroatoms selected from N, O, S, P, and Si, and remaining carbons in one functional group.

As used herein, “an aryl group” refers to a group including at least one hydrocarbon aromatic moiety, and all elements of the hydrocarbon aromatic moiety have p-orbitals which form conjugation, for example a phenyl group, a naphthyl group, and the like, two or more hydrocarbon aromatic moieties may be linked by a sigma bond and may be, for example a biphenyl group, a terphenyl group, a quarterphenyl group, and the like, and two or more hydrocarbon aromatic moieties are fused directly or indirectly to provide a non-aromatic fused ring, for example a fluorenyl group.

The aryl group may include a monocyclic, polycyclic, or fused ring polycyclic (i.e., rings sharing adjacent pairs of carbon atoms) functional group.

As used herein, “a heterocyclic group” is a generic concept of a heteroaryl group, and may include at least one heteroatom selected from N, O, S, P, and Si instead of carbon (C) in a cyclic compound such as an aryl group, a cycloalkyl group, a fused ring thereof, or a combination thereof. When the heterocyclic group is a fused ring, the entire ring or each ring of the heterocyclic group may include one or more heteroatoms.

For example, “a heteroaryl group” may refer to an aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted triphenylene group, a substituted or unsubstituted perylenyl group, a substituted or unsubstituted fluorenyl group, a substituted or unsubstituted indenyl group, or a combination thereof.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzothiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, or a substituted or unsubstituted dibenzothiophenyl group, or a combination thereof.

In the present specification, hole characteristics refer to an ability to donate an electron to form a hole when an electric field is applied and that a hole formed in the anode may be easily injected into the light emitting layer and transported in the light emitting layer due to conductive characteristics according to a highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to a lowest unoccupied molecular orbital (LUMO) level.

Hereinafter, a compound for an organic optoelectronic device according to an example embodiment is described.

A compound for an organic optoelectronic device according to an example embodiment is represented by Chemical Formula 1.

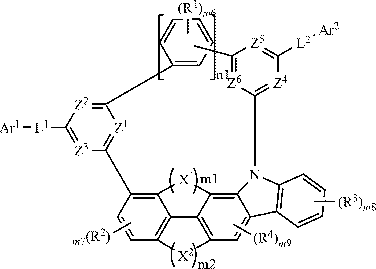

In an example embodiment of Chemical Formula 1,
m1 and m2 are each independently an integer of 0 or 1,
X¹ and X² are each independently NR^{g}, O, S, CR^{a}R^{b}, or SiR^{c}R^{d},
n1 is an integer of 1 to 5,
Z¹ to Z⁶ are each independently N or CR^{e},
at least two of Z¹ to Z³ are N; at least two of Z⁴ to Z⁶ are N; or at least two of Z¹ to Z³ are N and at least two of Z⁴ to Z⁶ are N,
R^{a}, R^{b}, R^{c}, R^{d} and R^{g} are each independently a substituted or unsubstituted C1 to C30 alkyl group, or a substituted or unsubstituted C6 to C30 aryl group,
R^{e} and R¹ toR⁴ are each independently hydrogen, deuterium, a halogen, a substituted or unsubstituted C1 to C30 alkyl group, or a substituted or unsubstituted C6 to C30 aryl group,
m6 and m8 are each independently an integer of 1 to 4,
m7 is an integer of 1 to 4,
m9 is an integer of 1 to 3,
Ar¹ and Ar² are each independently a substituted or unsubstituted C6 to C30 aryl group or a substituted or unsubstituted C2 to C30 heterocyclic group, and
L¹ and L² are each independently a single bond, a substituted or unsubstituted C6 to C30 arylene group, or a substituted or unsubstituted C2 to C30 heterocyclic group.

The compound represented by Chemical Formula 1 is a macro cycle-type bipolar compound. Without being bound by theory, it is believed that the compound represented by Chemical Formula 1 has a steric structural disorder compared to a structure that does not include a macro cycle skeleton, so that a degree of molecular freedom is reduced in the compound represented by Chemical Formula 1, and thus, molecules are arranged in a certain direction, an intermolecular orientation is increased to increase intermolecular electron and hole mobility and to exhibit a relatively high efficiency. In addition, the degree of molecular freedom is suppressed by the macrocycle skeleton, which shows a small reorganization with a small difference in molecular structure and energy between an excited state and a ground state, and exhibits a longer life-span compared to the non-macro cycle.

In an example embodiment, Chemical Formula 1 may be represented by, e.g., any one of Chemical Formula 1A to Chemical Formula 1D.

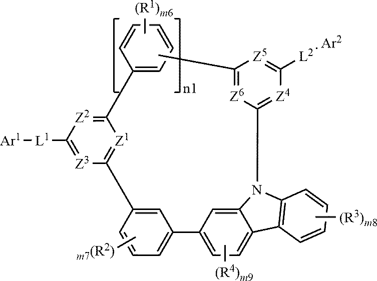

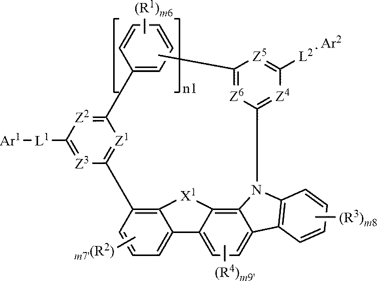

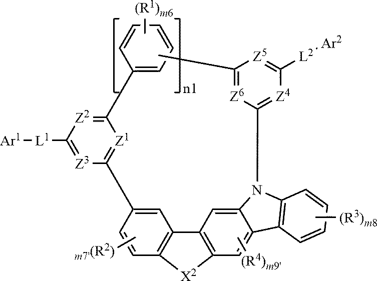

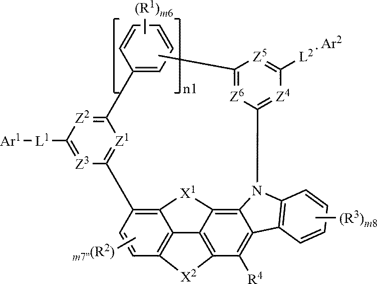

In Chemical Formula 1A to Chemical Formula 1D,
Ar¹, Ar², L¹, L², X¹, X², Z¹ toZ⁶, n1, m6 to m9, and R¹ to R⁴ are the same as described above,
m7′ is an integer of 1 to 3,
m7″ is an integer of 1 or 2, and
m9′ is an integer of 1 or 2.

In an example embodiment, Chemical Formula 1 may be represented by any one of Chemical Formula 1-I, Chemical Formula 1-II, and Chemical Formula 1-III.

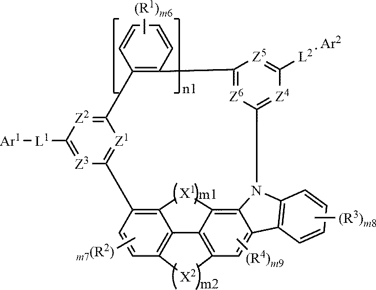

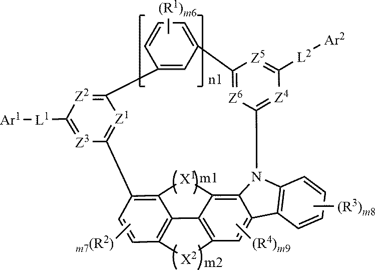

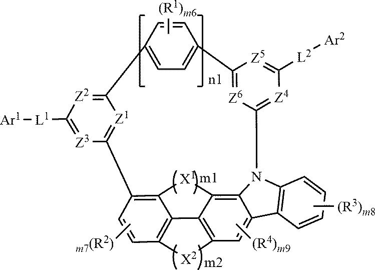

In an example embodiment, in Chemical Formula 1-I, Chemical Formula 1-II, and Chemical Formula 1-III,

Ar¹, Ar², L¹, L², X¹, X², m1, m2, m6 to m9, Z¹ toZ⁶, n1, and R¹ to R⁴ are the same as described above.

In an example embodiment, n1 may be one of an integer of 1 to 3, and for example, Chemical Formula 1 may be represented by any one of Chemical Formula 1-IIa, Chemical Formula 1-IIb, and Chemical Formula 1-IIc.

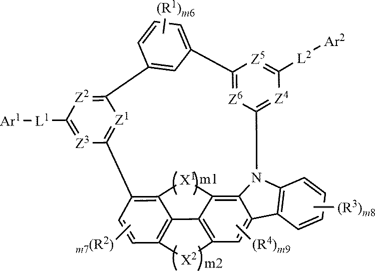

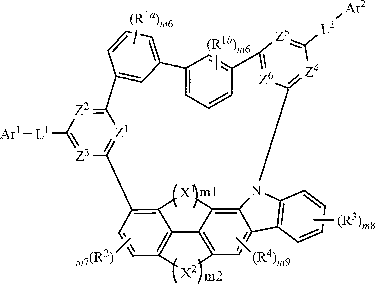

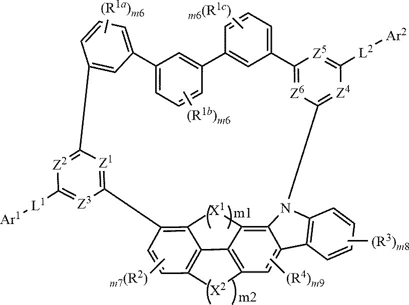

In an example embodiment, in Chemical Formula 1-IIa, Chemical Formula 1-IIb, and Chemical Formula 1-IIc,
Ar¹, Ar², L¹, L², X¹, X², m1, m2, m6 to m9, Z¹ toZ⁶, and R¹ to R⁴ are the same as described above, and
R^{1a}, R^{1b}, and R^{1c} have the same definitions as R¹ described above.

In an example embodiment, m1 and m2 may each be 0.

In an example embodiment, m1 may be 0 and m2 may be 1. In this case, X² may be N R^{g}, O, S, CR^{a}R^{b}, or SiR^{c}R^{d}, and R^{a}, R^{b}, R^{c}, R^{d} and R^{g} may each independently be a substituted or unsubstituted C1 to C10 alkyl group or a substituted or unsubstituted C6 to C12 aryl group.

In an example embodiment, m2 may be 0 and m1 may be 1. In this case, X¹ may be N R^{g}, O, S, CR^{a}R^{b}, or SiR^{c}R^{d}, and R^{a}, R^{b}, R^{c}, R^{d} and R^{g} may each independently be a substituted or unsubstituted C1 to C10 alkyl group or a substituted or unsubstituted C6 to C12 aryl group.

In an example embodiment, m1 and m2 may each be 1. In this case, X¹ and X² may each independently be NR^{g}, O, S, CR^{a}R^{b}, or SiR^{c}R^{d}, and R^{a}, R^{b}, R^{c}, R^{d} and R^{g} may each independently be a substituted or unsubstituted C1 to C10 alkyl group or a substituted or unsubstituted C6 to C12 aryl group. For example, the R^{g} may be a phenyl group.

In an example embodiment, X¹ and X² may each independently be O or S.

In an example embodiment, Z¹ to Z³ may each be N, and Z⁴ to Z⁶ may each independently be CR^{e}. Each R^{e} is the same as described above, and may be the same or different from each other.

In an example embodiment, Z⁴ to Z⁶ may each be N, and Z¹ to Z³ may each independently be CR^{e}. Each R^{e} is the same as described above, and may be the same or different from each other.

In an example embodiment, Z¹ to Z⁶ may each be N.

In an example embodiment, R^{e} and R¹ to R⁴ may each independently be hydrogen, deuterium, a halogen group, a substituted or unsubstituted C1 to C10 alkyl group, or a substituted or unsubstituted C6 to C12 aryl group. For example, R^{e} and R¹ toR⁴ may each independently be hydrogen, a substituted or unsubstituted C1 to C5 alkyl group, or a substituted or unsubstituted phenyl group.

In an example embodiment, Ar¹ and Ar² may each independently be a substituted or unsubstituted phenyl group, a substituted or unsubstituted biphenyl group, a substituted or unsubstituted terphenyl group, a substituted or unsubstituted naphthyl group, a substituted or unsubstituted fluorenyl group, a substituted or unsubstituted triphenylene group, a substituted or unsubstituted dibenzofuranyl group, a substituted or unsubstituted dibenzothiophenyl group, or a substituted or unsubstituted dibenzosilolyl group.

In an example embodiment, L¹ and L² may each independently be a single bond, or a substituted or unsubstituted C6 to C18 arylene group.

In an example embodiment, *-L¹-Ar¹ and *-L²-Ar² may be each independently selected from the groups of Group I.

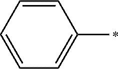

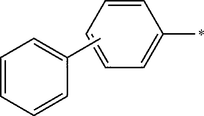

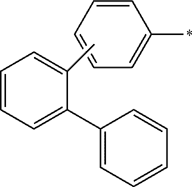

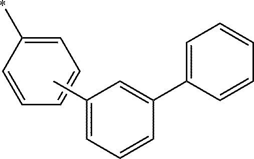

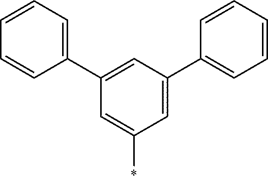 [SMILES: *c1cc(-c2ccccc2)cc(-c2ccccc2)c1]

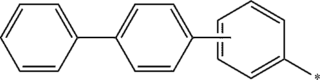

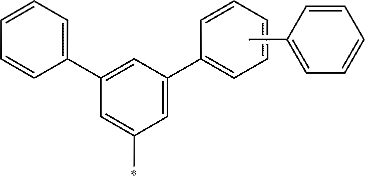

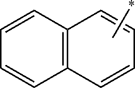

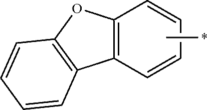

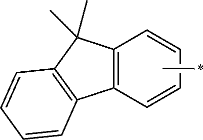

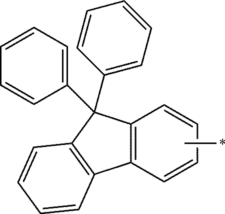

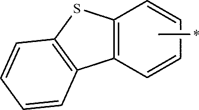

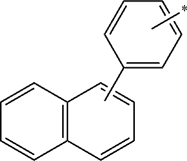

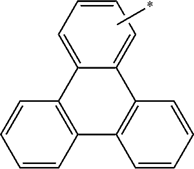

In Group I,
is a linking point, and
the groups of Group I may be unsubstituted or substituted with other substituents. The substituents are the same as described above. For example, the substituents may be selected from deuterium, a C1 to C10 alkyl group, and a C6 to C12 aryl group.

Examples of the compound for the organic optoelectronic device represented by Chemical Formula 1 may include the compounds of Group 1.

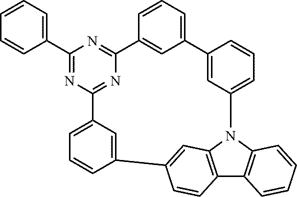 [SMILES: c1ccc(-c2nc3nc(n2)-c2cccc(c2)-c2ccc4c5ccccc5n(c4c2)-c2cccc(c2)-c2cccc-3c2)cc1]

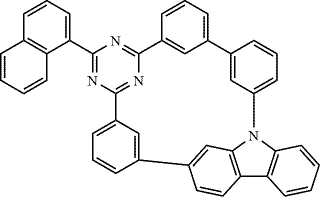 [SMILES: c1cc2cc(c1)-c1nc(nc(-c3cccc4ccccc34)n1)-c1cccc(c1)-c1ccc3c4ccccc4n(c3c1)-c1cccc-2c1]

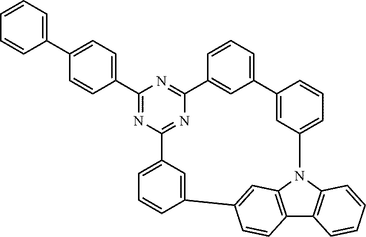 [SMILES: c1ccc(-c2ccc(-c3nc4nc(n3)-c3cccc(c3)-c3ccc5c6ccccc6n(c5c3)-c3cccc(c3)-c3cccc-4c3)cc2)cc1]

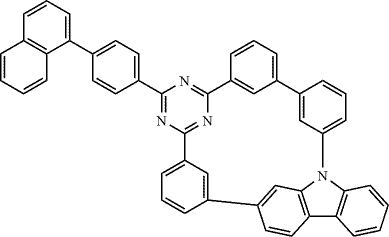 [SMILES: c1cc2cc(c1)-c1nc(-c3ccc(-c4cccc5ccccc45)cc3)nc(n1)-c1cccc(c1)-c1ccc3c4ccccc4n(c3c1)-c1cccc-2c1]

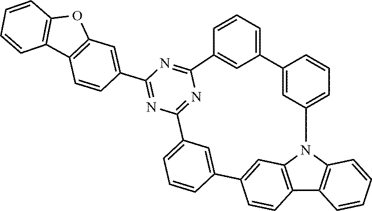 [SMILES: c1cc2cc(c1)-c1nc(nc(-c3ccc4c(c3)oc3ccccc34)n1)-c1cccc(c1)-c1ccc3c4ccccc4n(c3c1)-c1cccc-2c1]

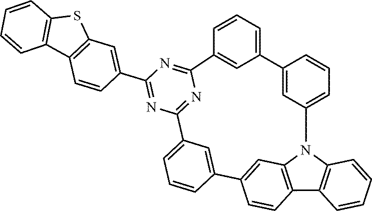 [SMILES: c1cc2cc(c1)-c1nc(nc(-c3ccc4c(c3)sc3ccccc34)n1)-c1cccc(c1)-c1ccc3c4ccccc4n(c3c1)-c1cccc-2c1]

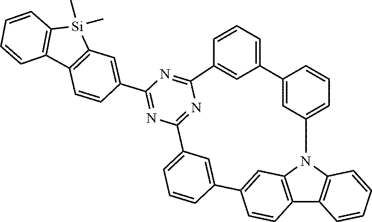 [SMILES: C[Si]1(C)c2ccccc2-c2ccc(-c3nc4nc(n3)-c3cccc(c3)-c3ccc5c6ccccc6n(c5c3)-c3cccc(c3)-c3cccc-4c3)cc21]

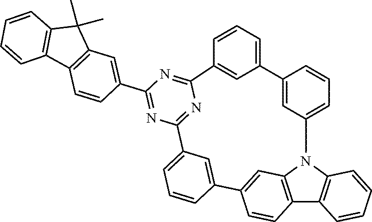 [SMILES: CC1(C)c2ccccc2-c2ccc(-c3nc4nc(n3)-c3cccc(c3)-c3ccc5c6ccccc6n(c5c3)-c3cccc(c3)-c3cccc-4c3)cc21]

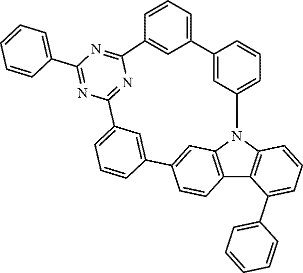 [SMILES: c1ccc(-c2nc3nc(n2)-c2cccc(c2)-c2ccc4c5c(-c6ccccc6)cccc5n(c4c2)-c2cccc(c2)-c2cccc-3c2)cc1]

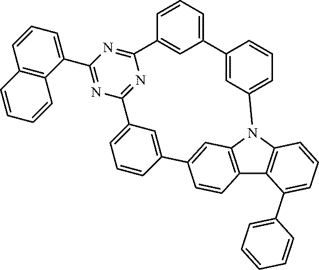 [SMILES: c1ccc(-c2cccc3c2c2ccc4cc2n3-c2cccc(c2)-c2cccc(c2)-c2nc(nc(-c3cccc5ccccc35)n2)-c2cccc-4c2)cc1]

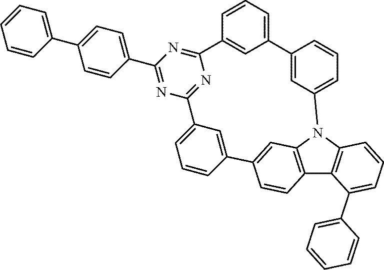 [SMILES: c1ccc(-c2ccc(-c3nc4nc(n3)-c3cccc(c3)-c3ccc5c6c(-c7ccccc7)cccc6n(c5c3)-c3cccc(c3)-c3cccc-4c3)cc2)cc1]

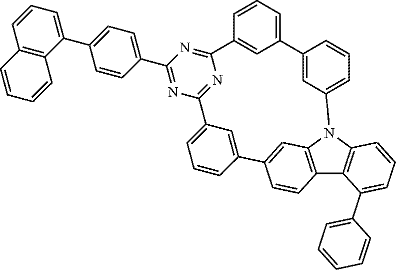 [SMILES: c1ccc(-c2cccc3c2c2ccc4cc2n3-c2cccc(c2)-c2cccc(c2)-c2nc(-c3ccc(-c5cccc6ccccc56)cc3)nc(n2)-c2cccc-4c2)cc1]

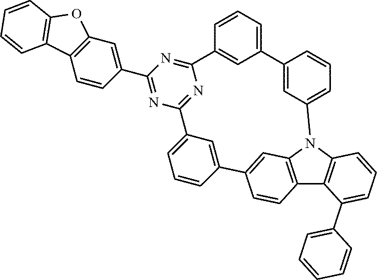 [SMILES: c1ccc(-c2cccc3c2c2ccc4cc2n3-c2cccc(c2)-c2cccc(c2)-c2nc(nc(-c3ccc5c(c3)oc3ccccc35)n2)-c2cccc-4c2)cc1]

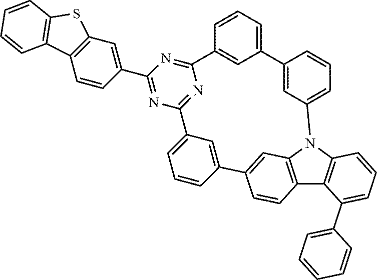 [SMILES: c1ccc(-c2cccc3c2c2ccc4cc2n3-c2cccc(c2)-c2cccc(c2)-c2nc(nc(-c3ccc5c(c3)sc3ccccc35)n2)-c2cccc-4c2)cc1]

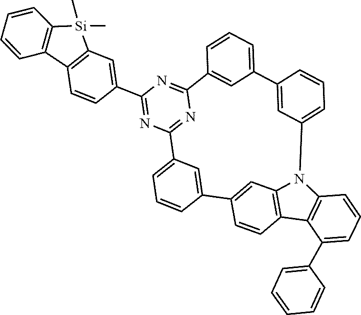 [SMILES: C[Si]1(C)c2ccccc2-c2ccc(-c3nc4nc(n3)-c3cccc(c3)-c3ccc5c6c(-c7ccccc7)cccc6n(c5c3)-c3cccc(c3)-c3cccc-4c3)cc21]

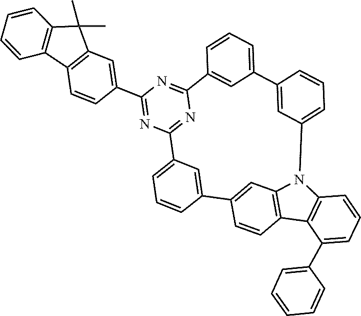 [SMILES: CC1(C)c2ccccc2-c2ccc(-c3nc4nc(n3)-c3cccc(c3)-c3ccc5c6c(-c7ccccc7)cccc6n(c5c3)-c3cccc(c3)-c3cccc-4c3)cc21]

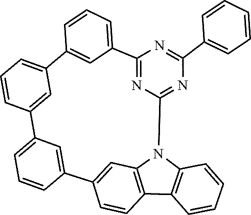 [SMILES: c1ccc(-c2nc3nc(n2)-n2c4ccccc4c4ccc(cc42)-c2cccc(c2)-c2cccc(c2)-c2cccc-3c2)cc1]

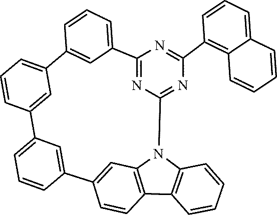 [SMILES: c1cc2cc(c1)-c1cccc(c1)-c1nc(-c3cccc4ccccc34)nc(n1)-n1c3ccccc3c3ccc(cc31)-c1cccc-2c1]

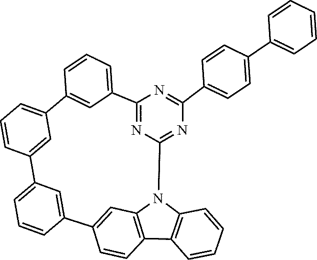 [SMILES: c1ccc(-c2ccc(-c3nc4nc(n3)-n3c5ccccc5c5ccc(cc53)-c3cccc(c3)-c3cccc(c3)-c3cccc-4c3)cc2)cc1]

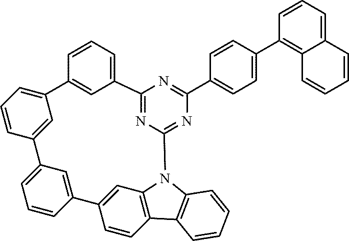 [SMILES: c1cc2cc(c1)-c1cccc(c1)-c1nc(-c3ccc(-c4cccc5ccccc45)cc3)nc(n1)-n1c3ccccc3c3ccc(cc31)-c1cccc-2c1]

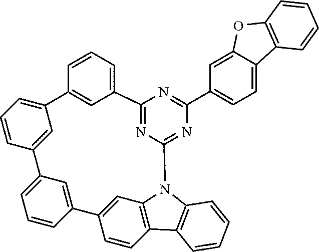 [SMILES: c1cc2cc(c1)-c1cccc(c1)-c1nc(-c3ccc4c(c3)oc3ccccc34)nc(n1)-n1c3ccccc3c3ccc(cc31)-c1cccc-2c1]

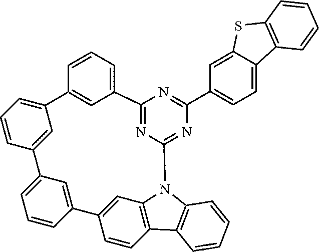 [SMILES: c1cc2cc(c1)-c1cccc(c1)-c1nc(-c3ccc4c(c3)sc3ccccc34)nc(n1)-n1c3ccccc3c3ccc(cc31)-c1cccc-2c1]

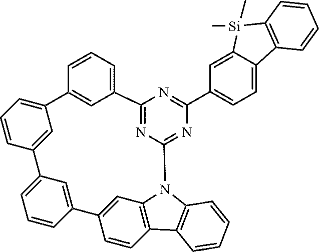 [SMILES: C[Si]1(C)c2ccccc2-c2ccc(-c3nc4nc(n3)-n3c5ccccc5c5ccc(cc53)-c3cccc(c3)-c3cccc(c3)-c3cccc-4c3)cc21]

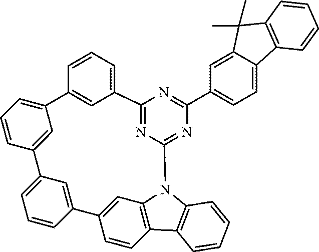 [SMILES: CC1(C)c2ccccc2-c2ccc(-c3nc4nc(n3)-n3c5ccccc5c5ccc(cc53)-c3cccc(c3)-c3cccc(c3)-c3cccc-4c3)cc21]

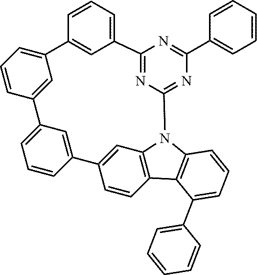 [SMILES: c1ccc(-c2nc3nc(n2)-n2c4cc(ccc4c4c(-c5ccccc5)cccc42)-c2cccc(c2)-c2cccc(c2)-c2cccc-3c2)cc1]

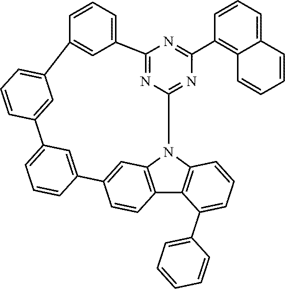 [SMILES: c1ccc(-c2cccc3c2c2ccc4cc2n3-c2nc(nc(-c3cccc5ccccc35)n2)-c2cccc(c2)-c2cccc(c2)-c2cccc-4c2)cc1]

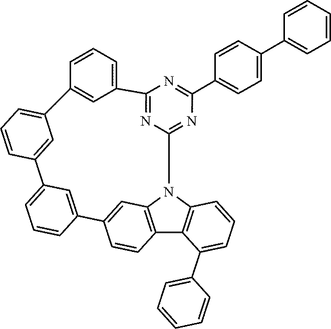 [SMILES: c1ccc(-c2ccc(-c3nc4nc(n3)-n3c5cc(ccc5c5c(-c6ccccc6)cccc53)-c3cccc(c3)-c3cccc(c3)-c3cccc-4c3)cc2)cc1]

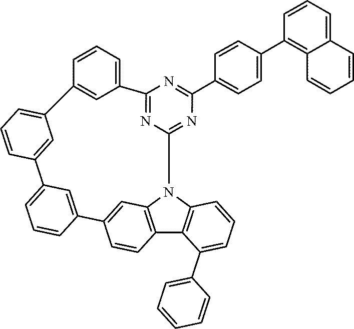 [SMILES: c1ccc(-c2cccc3c2c2ccc4cc2n3-c2nc(-c3ccc(-c5cccc6ccccc56)cc3)nc(n2)-c2cccc(c2)-c2cccc(c2)-c2cccc-4c2)cc1]

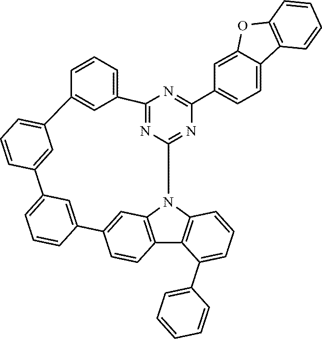 [SMILES: c1ccc(-c2cccc3c2c2ccc4cc2n3-c2nc(nc(-c3ccc5c(c3)oc3ccccc35)n2)-c2cccc(c2)-c2cccc(c2)-c2cccc-4c2)cc1]

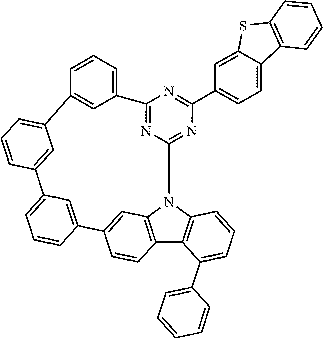 [SMILES: c1ccc(-c2cccc3c2c2ccc4cc2n3-c2nc(nc(-c3ccc5c(c3)sc3ccccc35)n2)-c2cccc(c2)-c2cccc(c2)-c2cccc-4c2)cc1]

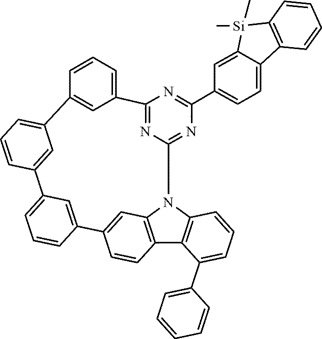 [SMILES: C[Si]1(C)c2ccccc2-c2ccc(-c3nc4nc(n3)-n3c5cc(ccc5c5c(-c6ccccc6)cccc53)-c3cccc(c3)-c3cccc(c3)-c3cccc-4c3)cc21]

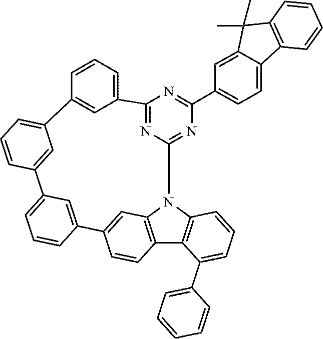 [SMILES: CC1(C)c2ccccc2-c2ccc(-c3nc4nc(n3)-n3c5cc(ccc5c5c(-c6ccccc6)cccc53)-c3cccc(c3)-c3cccc(c3)-c3cccc-4c3)cc21]

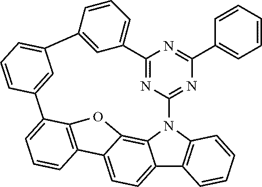 [SMILES: c1ccc(-c2nc3nc(n2)n2c4ccccc4c4ccc5c6cccc(c7cccc(c7)c7cccc3c7)c6oc5c42)cc1]

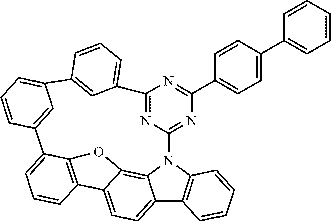 [SMILES: c1ccc(-c2ccc(-c3nc4nc(n3)n3c5ccccc5c5ccc6c7cccc(c8cccc(c8)c8cccc4c8)c7oc6c53)cc2)cc1]

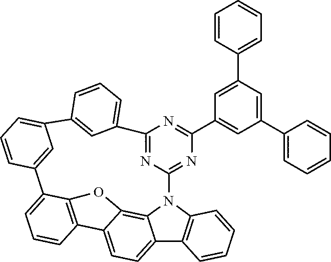 [SMILES: c1ccc(-c2cc(-c3ccccc3)cc(-c3nc4nc(n3)n3c5ccccc5c5ccc6c7cccc(c8cccc(c8)c8cccc4c8)c7oc6c53)c2)cc1]

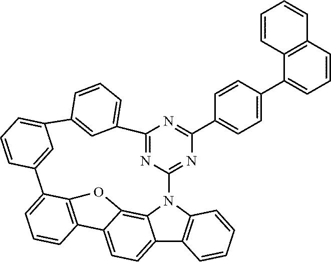 [SMILES: c1cc2cc(c1)c1nc(-c3ccc(-c4cccc5ccccc45)cc3)nc(n1)n1c3ccccc3c3ccc4c5cccc(c6cccc2c6)c5oc4c31]

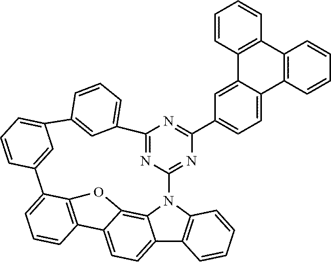 [SMILES: c1cc2cc(c1)c1nc(-c3ccc4c5ccccc5c5ccccc5c4c3)nc(n1)n1c3ccccc3c3ccc4c5cccc(c6cccc2c6)c5oc4c31]

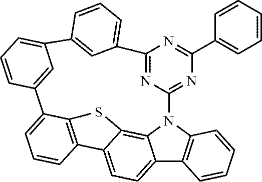 [SMILES: c1ccc(-c2nc3nc(n2)n2c4ccccc4c4ccc5c6cccc(c7cccc(c7)c7cccc3c7)c6sc5c42)cc1]

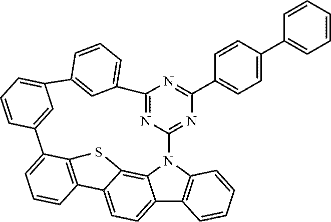 [SMILES: c1ccc(-c2ccc(-c3nc4nc(n3)n3c5ccccc5c5ccc6c7cccc(c8cccc(c8)c8cccc4c8)c7sc6c53)cc2)cc1]

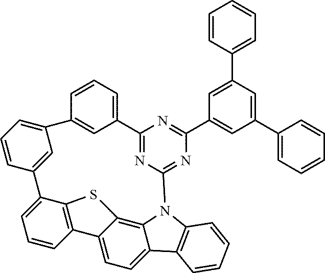 [SMILES: c1ccc(-c2cc(-c3ccccc3)cc(-c3nc4nc(n3)n3c5ccccc5c5ccc6c7cccc(c8cccc(c8)c8cccc4c8)c7sc6c53)c2)cc1]

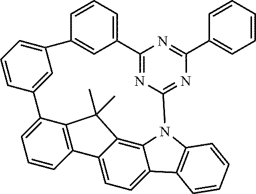 [SMILES: CC1(C)c2c3cccc2-c2ccc4c5ccccc5n(c4c21)-c1nc(-c2ccccc2)nc(n1)-c1cccc(c1)-c1cccc-3c1]

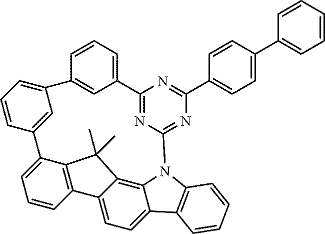 [SMILES: CC1(C)c2c3cccc2-c2ccc4c5ccccc5n(c4c21)-c1nc(-c2ccc(-c4ccccc4)cc2)nc(n1)-c1cccc(c1)-c1cccc-3c1]

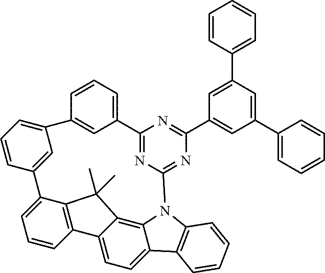 [SMILES: CC1(C)c2c3cccc2-c2ccc4c5ccccc5n(c4c21)-c1nc(nc(-c2cc(-c4ccccc4)cc(-c4ccccc4)c2)n1)-c1cccc(c1)-c1cccc-3c1]

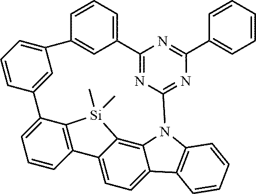 [SMILES: C[Si]1(C)c2c3cccc2-c2ccc4c5ccccc5n(c4c21)-c1nc(-c2ccccc2)nc(n1)-c1cccc(c1)-c1cccc-3c1]

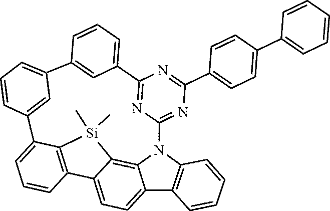 [SMILES: C[Si]1(C)c2c3cccc2-c2ccc4c5ccccc5n(c4c21)-c1nc(-c2ccc(-c4ccccc4)cc2)nc(n1)-c1cccc(c1)-c1cccc-3c1]

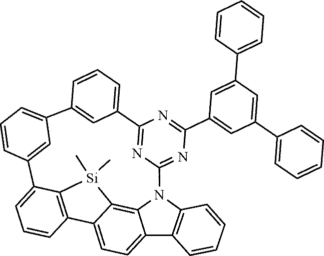 [SMILES: C[Si]1(C)c2c3cccc2-c2ccc4c5ccccc5n(c4c21)-c1nc(nc(-c2cc(-c4ccccc4)cc(-c4ccccc4)c2)n1)-c1cccc(c1)-c1cccc-3c1]

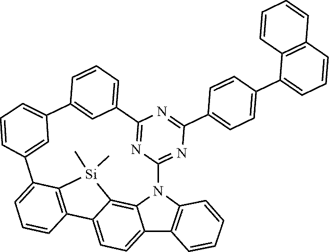 [SMILES: C[Si]1(C)c2c3cccc2-c2ccc4c5ccccc5n(c4c21)-c1nc(-c2ccc(-c4cccc5ccccc45)cc2)nc(n1)-c1cccc(c1)-c1cccc-3c1]

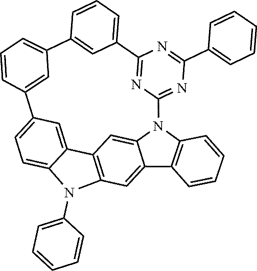 [SMILES: c1ccc(-c2nc3nc(n2)n2c4ccccc4c4cc5c(cc42)c2cc(ccc2n5-c2ccccc2)c2cccc(c2)c2cccc3c2)cc1]

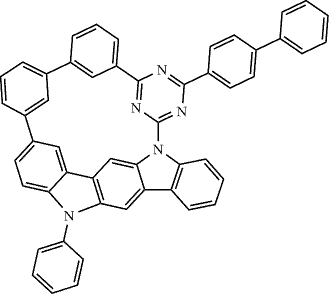 [SMILES: c1ccc(-c2ccc(-c3nc4nc(n3)n3c5ccccc5c5cc6c(cc53)c3cc(ccc3n6-c3ccccc3)c3cccc(c3)c3cccc4c3)cc2)cc1]

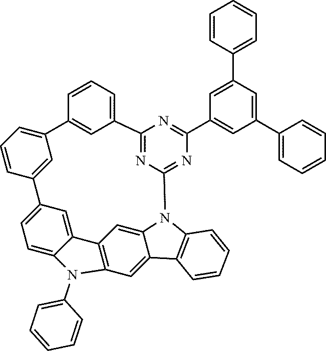 [SMILES: c1ccc(-c2cc(-c3ccccc3)cc(-c3nc4nc(n3)n3c5ccccc5c5cc6c(cc53)c3cc(ccc3n6-c3ccccc3)c3cccc(c3)c3cccc4c3)c2)cc1]

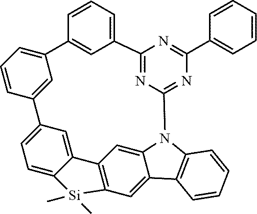 [SMILES: C[Si]1(C)c2ccc3cc2-c2cc4c(cc21)c1ccccc1n4-c1nc(-c2ccccc2)nc(n1)-c1cccc(c1)-c1cccc-3c1]

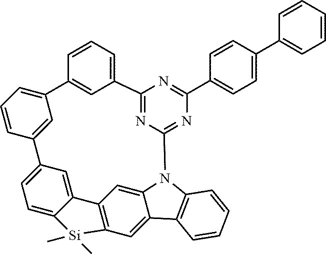 [SMILES: C[Si]1(C)c2ccc3cc2-c2cc4c(cc21)c1ccccc1n4-c1nc(-c2ccc(-c4ccccc4)cc2)nc(n1)-c1cccc(c1)-c1cccc-3c1]

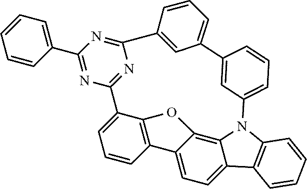 [SMILES: c1ccc(-c2nc3nc(n2)c2cccc4c5ccc6c7ccccc7n(c7cccc(c7)c7cccc3c7)c6c5oc24)cc1]

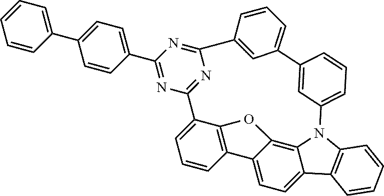 [SMILES: c1ccc(-c2ccc(-c3nc4nc(n3)c3cccc5c6ccc7c8ccccc8n(c8cccc(c8)c8cccc4c8)c7c6oc35)cc2)cc1]

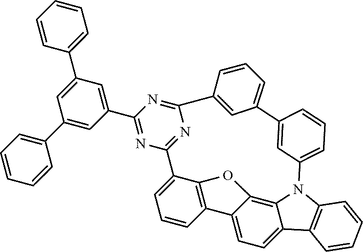 [SMILES: c1ccc(-c2cc(-c3ccccc3)cc(-c3nc4nc(n3)c3cccc5c6ccc7c8ccccc8n(c8cccc(c8)c8cccc4c8)c7c6oc35)c2)cc1]

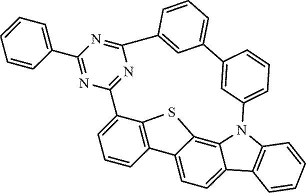 [SMILES: c1ccc(-c2nc3nc(n2)c2cccc4c5ccc6c7ccccc7n(c7cccc(c7)c7cccc3c7)c6c5sc24)cc1]

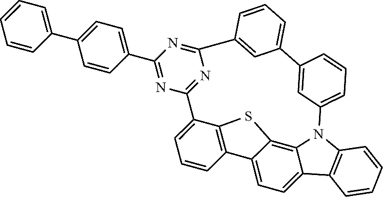 [SMILES: c1ccc(-c2ccc(-c3nc4nc(n3)c3cccc5c6ccc7c8ccccc8n(c8cccc(c8)c8cccc4c8)c7c6sc35)cc2)cc1]

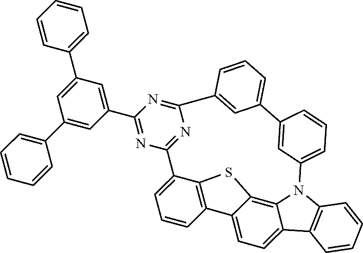 [SMILES: c1ccc(-c2cc(-c3ccccc3)cc(-c3nc4nc(n3)c3cccc5c6ccc7c8ccccc8n(c8cccc(c8)c8cccc4c8)c7c6sc35)c2)cc1]

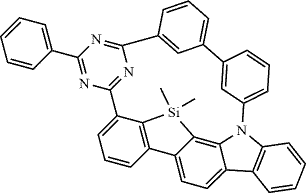 [SMILES: C[Si]1(C)c2c3cccc2-c2ccc4c5ccccc5n(c4c21)-c1cccc(c1)-c1cccc(c1)-c1nc(-c2ccccc2)nc-3n1]

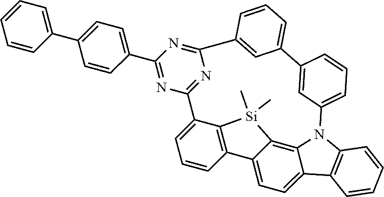 [SMILES: C[Si]1(C)c2c3cccc2-c2ccc4c5ccccc5n(c4c21)-c1cccc(c1)-c1cccc(c1)-c1nc(-c2ccc(-c4ccccc4)cc2)nc-3n1]

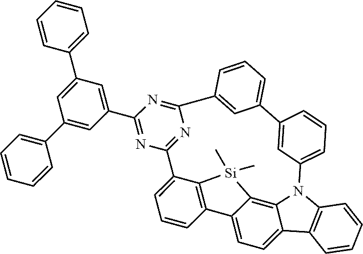 [SMILES: C[Si]1(C)c2c3cccc2-c2ccc4c5ccccc5n(c4c21)-c1cccc(c1)-c1cccc(c1)-c1nc(-c2cc(-c4ccccc4)cc(-c4ccccc4)c2)nc-3n1]

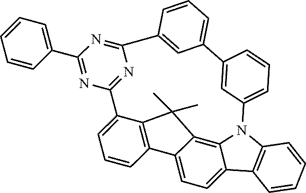 [SMILES: CC1(C)c2c3cccc2-c2ccc4c5ccccc5n(c4c21)-c1cccc(c1)-c1cccc(c1)-c1nc(-c2ccccc2)nc-3n1]

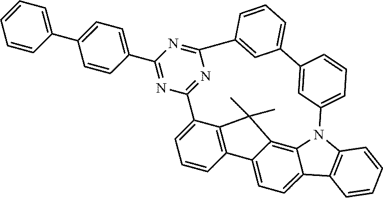 [SMILES: CC1(C)c2c3cccc2-c2ccc4c5ccccc5n(c4c21)-c1cccc(c1)-c1cccc(c1)-c1nc(-c2ccc(-c4ccccc4)cc2)nc-3n1]

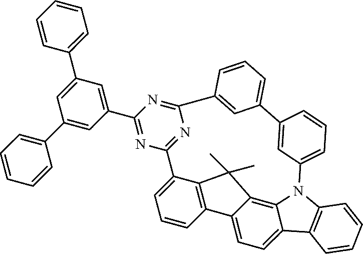 [SMILES: CC1(C)c2c3cccc2-c2ccc4c5ccccc5n(c4c21)-c1cccc(c1)-c1cccc(c1)-c1nc(-c2cc(-c4ccccc4)cc(-c4ccccc4)c2)nc-3n1]

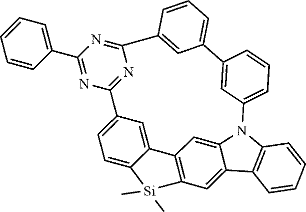 [SMILES: C[Si]1(C)c2ccc3cc2-c2cc4c(cc21)c1ccccc1n4-c1cccc(c1)-c1cccc(c1)-c1nc(-c2ccccc2)nc-3n1]

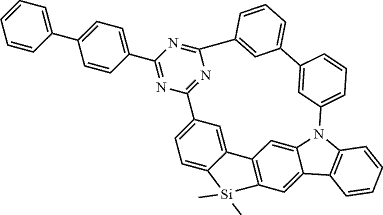 [SMILES: C[Si]1(C)c2ccc3cc2-c2cc4c(cc21)c1ccccc1n4-c1cccc(c1)-c1cccc(c1)-c1nc(-c2ccc(-c4ccccc4)cc2)nc-3n1]

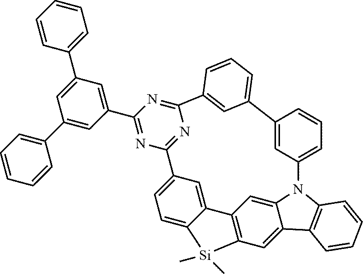 [SMILES: C[Si]1(C)c2ccc3cc2-c2cc4c(cc21)c1ccccc1n4-c1cccc(c1)-c1cccc(c1)-c1nc(-c2cc(-c4ccccc4)cc(-c4ccccc4)c2)nc-3n1]

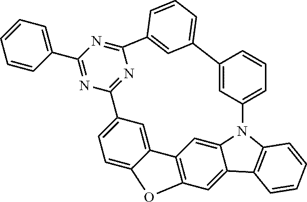 [SMILES: c1ccc(-c2nc3nc(n2)c2ccc4oc5cc6c7ccccc7n(c7cccc(c7)c7cccc3c7)c6cc5c4c2)cc1]

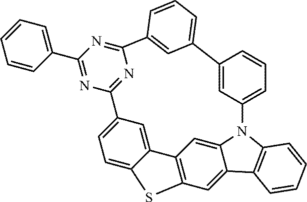 [SMILES: c1ccc(-c2nc3nc(n2)c2ccc4sc5cc6c7ccccc7n(c7cccc(c7)c7cccc3c7)c6cc5c4c2)cc1]

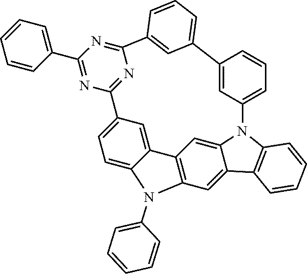 [SMILES: c1ccc(-c2nc3nc(n2)c2ccc4c(c2)c2cc5c(cc2n4-c2ccccc2)c2ccccc2n5c2cccc(c2)c2cccc3c2)cc1]

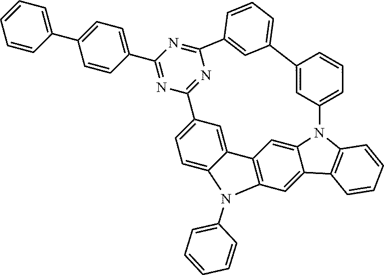 [SMILES: c1ccc(-c2ccc(-c3nc4nc(n3)c3ccc5c(c3)c3cc6c(cc3n5-c3ccccc3)c3ccccc3n6c3cccc(c3)c3cccc4c3)cc2)cc1]

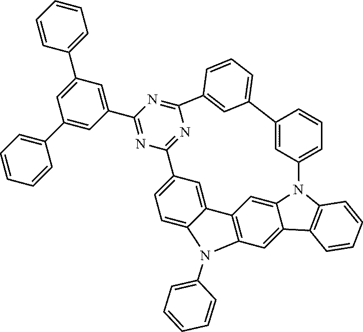 [SMILES: c1ccc(-c2cc(-c3ccccc3)cc(-c3nc4nc(n3)c3ccc5c(c3)c3cc6c(cc3n5-c3ccccc3)c3ccccc3n6c3cccc(c3)c3cccc4c3)c2)cc1]

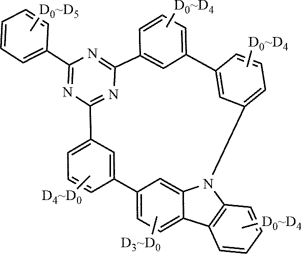

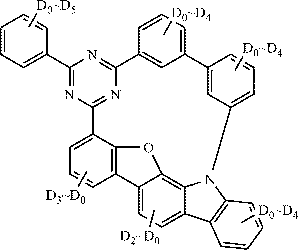

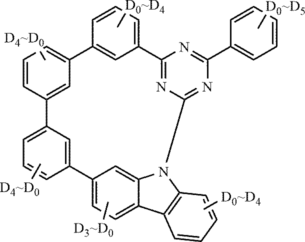

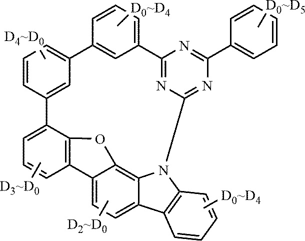

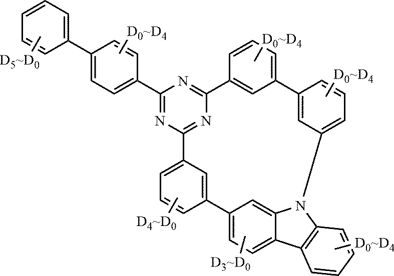

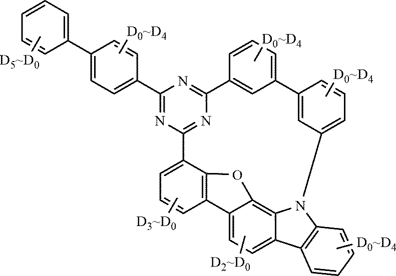

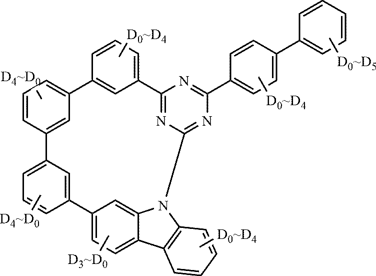

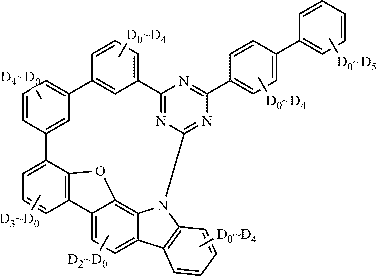

In Group 1,
D₀ refers to non-replacement of a hydrogen by deuterium,
D₁ refers to a replacement of a hydrogen by one deuterium,
D₂ refers to a replacement of hydrogens by two deuteriums,
D₃ refers to a replacement of hydrogens by three deuteriums,
D₄ refers to a replacement of hydrogens by four deuteriums,
D₅ refers to a replacement of hydrogens by five deuteriums,
D₀~D₂ refers to deuteriums can be substituted from 0 to 2,
D₀~D₃ refers to deuteriums can be substituted from 0 to 3,
D₀~D₄ refers to deuteriums can be substituted from 0 to 4, and
D₀~D₅ refers to deuteriums can be substituted from 0 to 5.

According to another example embodiment, a composition for an organic optoelectronic device includes a first compound and a second compound. The first compound may be the aforementioned compound for an organic optoelectronic device represented by Chemical Formula 1. The second compound may be a compound for an organic optoelectronic device represented by Chemical Formula 2; or a compound for an organic optoelectronic device represented by a combination of Chemical Formula 3 and Chemical Formula 4.

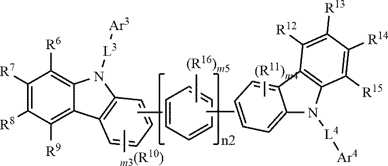

In an example embodiment of Chemical Formula 2,
Ar³ and Ar⁴ are each independently a substituted or unsubstituted C6 to C20 aryl group or a substituted or unsubstituted C2 to C30 heterocyclic group,
L³ and L⁴ are each independently a single bond or a substituted or unsubstituted C6 to C20 arylene group,
R⁶ to R¹⁶ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
m3 and m4 are each independently an integer from 1 to 3,
m5 is an integer from 1 to 4, and
n2 is an integer from 0 to 2;

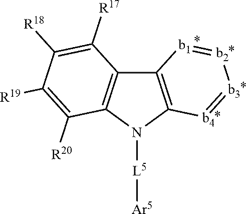

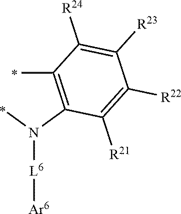

In an example embodiment of Chemical Formulas 3 and 4,
Ar⁵ and Ar⁶ are each independently a substituted or unsubstituted C6 to C20 aryl group or a substituted or unsubstituted C2 to C30 heterocyclic group,
adjacent two of b₁* to b₄* of Chemical Formula 3 are each a carbon (C) linked to * in Chemical Formula 4,
the other two not linked to * of Chemical Formula 4 of b₁* to b₄* in Chemical Formula 3 are each independently C- L^{d}-R^{f},
L^{d}, L⁵, and L⁶ are each independently a single bond or a substituted or unsubstituted C6 to C20 arylene group, and
R^{f} and R¹⁷ to R²⁴ are each independently hydrogen, deuterium, a cyano group, a halogen, a substituted or unsubstituted amine, a substituted or unsubstituted C1 to C30 alkyl group, a substituted or unsubstituted C6 to C30 aryl group, or a substituted or unsubstituted C2 to C30 heterocyclic group.

The second compound may be used in the light emitting layer together with the first compound to increase the mobility of charges and improve stability, thereby improving luminous efficiency and life-span characteristics.

In an example embodiment, in Chemical Formula 2,
Ar³ and Ar⁴ may each independently be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted carbazolyl group, a substituted or unsubstituted dibenzothiophenyl group, a substituted or unsubstituted dibenzofuranyl group, or a substituted or unsubstituted fluorenyl group,
L³ and L⁴ may each independently be a single bond, a substituted or unsubstituted phenylene group, or a substituted or unsubstituted biphenylene group,
R⁶ to R¹⁶ may each independently be hydrogen, deuterium, or a substituted or unsubstituted C6 to C12 aryl group, and
n2 may be 0 or 1.

The “substituted” in Chemical Formula 2 may mean that at least one hydrogen is replaced by deuterium, a C1 to C4 alkyl group, a C6 to C18 aryl group, or a C2 to C30 heteroaryl group.

In an example embodiment, Chemical Formula 2 may be represented by one of Chemical Formula 2-1 to Chemical Formula 2-15.

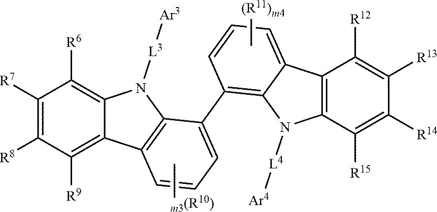

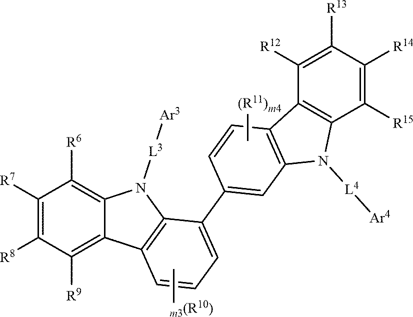

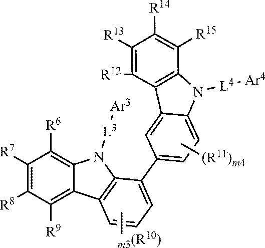

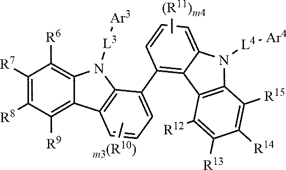

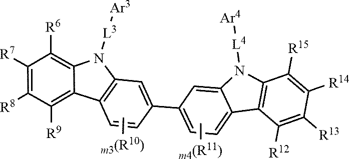

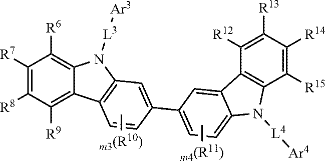

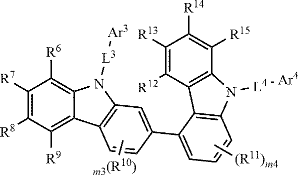

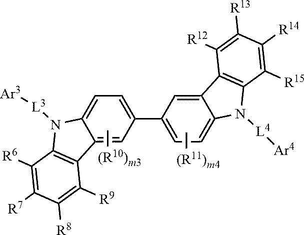

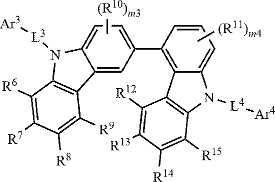

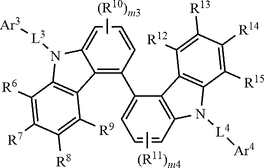

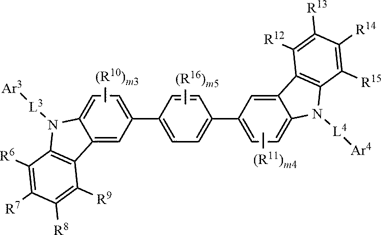

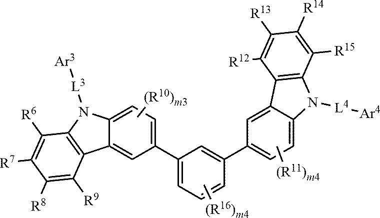

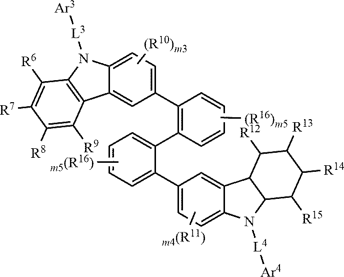

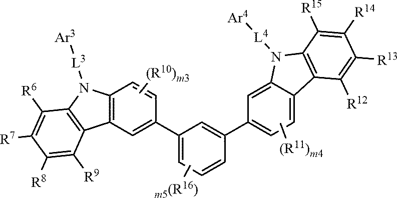

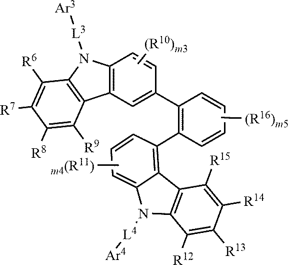

In Chemical Formula 2-1 to Chemical Formula 2-15,
R⁶ toR¹⁶ may each independently be hydrogen, deuterium, or a substituted or unsubstituted C6 to C12 aryl group, and
-L³-Ar³ and *-L⁴-Ar⁴ may each independently be one of the substituents of Group II.

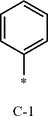

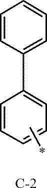

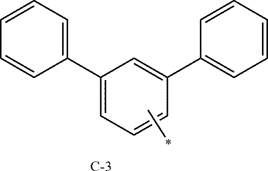

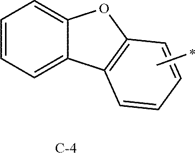

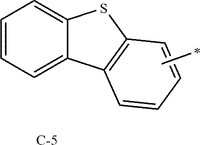

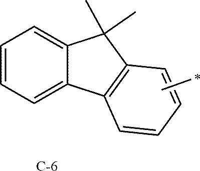

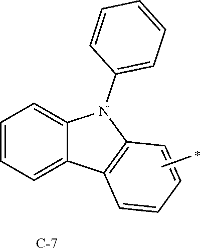

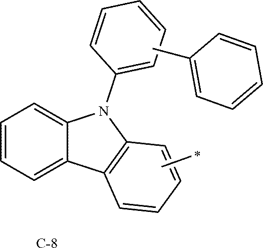

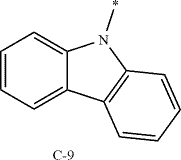

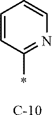

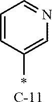

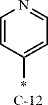

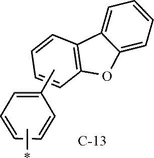

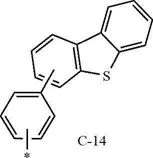

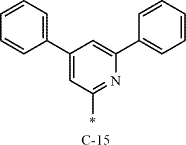

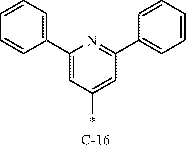

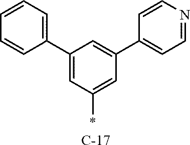

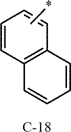

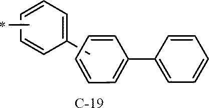

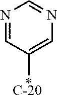

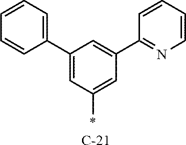

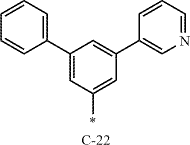

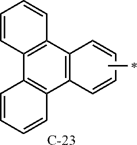

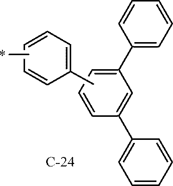

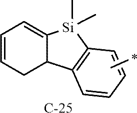

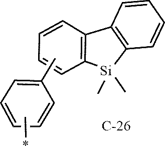

In Group II,
is a linking point, and
the groups of Group II may be unsubstitued or substituted with other substituents. The substituents are the same as described above. For example, the substituents may be selected from deuterium, a C1 to C4 alkyl group, a C6 to C18 aryl group, and a C2 to C30 heteroaryl group.

In an example embodiment, Chemical Formula 2 may be represented by Chemical Formula 2-8.

In an example embodiment, *-L³-Ar³ and *-L⁴-Ar⁴ of Chemical Formula 2-8 may be each independently selected from Group II, and may be, for example, any one of C-1, C-2, C-3, C-4, C-7, C-8, or C-9.

In an example embodiment, the second compound represented by the combination of Chemical Formula 3 and Chemical Formula 4 may be represented by any one of Chemical Formula 3A, Chemical Formula 3B, Chemical Formula 3C, Chemical Formula 3D, and Chemical Formula 3E.

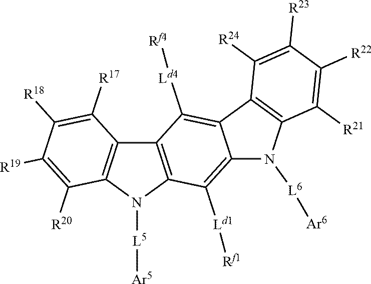

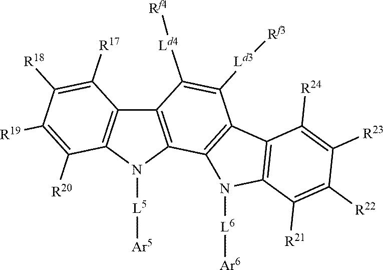

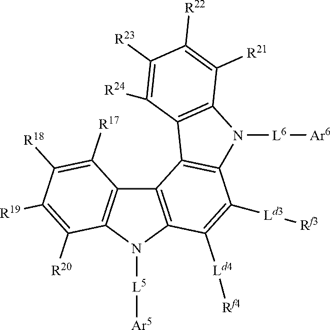

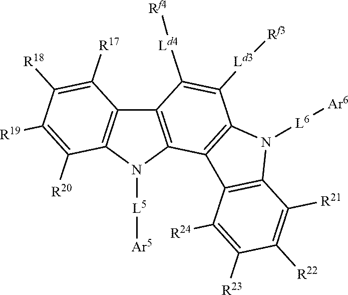

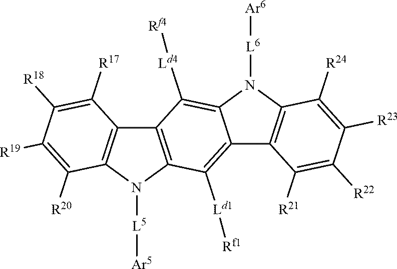

In Chemical Formula 3A to Chemical Formula 3E,
Ar⁵, Ar⁶, L⁵, L⁶, and R¹⁷ to R²⁴ are the same as described above,
L^{d1} to L^{d4} are the same as the definitions of L⁵ and L⁶ described above, and
R^{f1} to R^{f4} have the same definitions as R¹⁷ toR²⁴ described above.

In an example embodiment, in Chemical Formulas 3 and 4,
Ar⁵ and Ar⁶ may each independently be a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group, and
R^{f1} to R^{f4} and R¹⁷ to R¹⁴ may each independently be hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an example embodiment, in Chemical Formulas 3 and 4, *-L⁵-Ar⁵ and *-L⁶-Ar⁶ may each independently be selected from substituents of Group II.

In an example embodiment, R^{f1} to R^{f4} and R¹⁷ to R²⁴ may each independently be hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group. For example, R^{f1} to R^{f4} and R¹⁷ to R²⁴ may each independently be hydrogen, deuterium, a cyano group, or a substituted or unsubstituted phenyl group, and, in a specific example embodiment, the R^{f1} to R^{f4}, and R¹⁷ to R²⁴ may each independently be hydrogen, deuterium, or a phenyl group.

In an example embodiment, the second compound may be represented by Chemical Formula 2-8, wherein in Chemical Formula 2-8, Ar³ and Ar⁴ may each independently be a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group, L³ and L⁴ may each independently be a single bond, or a substituted or unsubstituted C6 to C20 arylene group, and R⁶ toR¹⁶ may each independently be hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In an example embodiment, the second compound may be represented by Chemical Formula 3C, wherein in Chemical Formula 3C, L^{f1} toL^{f4} may be a single bond, and L⁵ and L⁶ may each independently be a single bond, or substituted or unsubstituted a C6 to C12 arylene group, R¹⁷ to R²⁴ and R^{f1} to R^{f4} may each be hydrogen, deuterium, or a phenyl group, and Ar⁵ and Ar⁶ may each independently be a substituted or unsubstituted phenyl group, a substituted or unsubstituted biphenyl group, or a substituted or unsubstituted terphenyl group.

In an example embodiment, the second compound may be one selected from the compounds of Group 2. [Group 2]

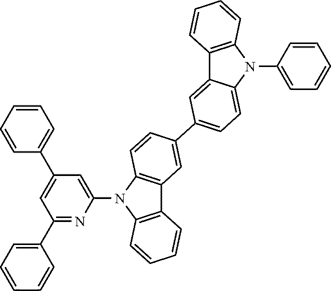 [SMILES: c1ccc(-c2cc(-c3ccccc3)nc(-n3c4ccccc4c4cc(-c5ccc6c(c5)c5ccccc5n6-c5ccccc5)ccc43)c2)cc1]

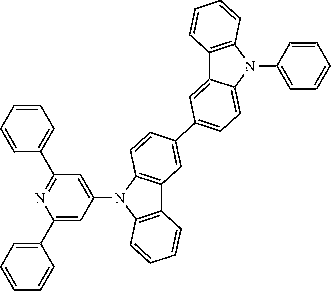 [SMILES: c1ccc(-c2cc(-n3c4ccccc4c4cc(-c5ccc6c(c5)c5ccccc5n6-c5ccccc5)ccc43)cc(-c3ccccc3)n2)cc1]

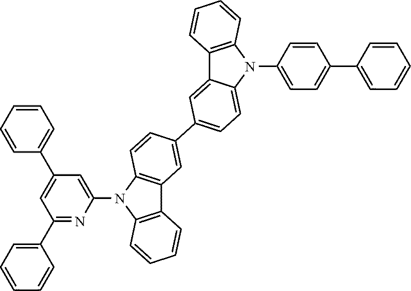 [SMILES: c1ccc(-c2ccc(-n3c4ccccc4c4cc(-c5ccc6c(c5)c5ccccc5n6-c5cc(-c6ccccc6)cc(-c6ccccc6)n5)ccc43)cc2)cc1]

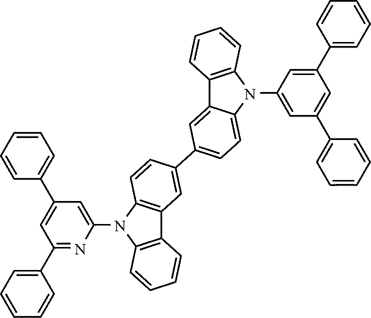 [SMILES: c1ccc(-c2cc(-c3ccccc3)cc(-n3c4ccccc4c4cc(-c5ccc6c(c5)c5ccccc5n6-c5cc(-c6ccccc6)cc(-c6ccccc6)n5)ccc43)c2)cc1]

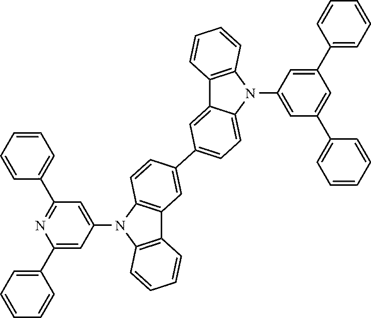 [SMILES: c1ccc(-c2cc(-c3ccccc3)cc(-n3c4ccccc4c4cc(-c5ccc6c(c5)c5ccccc5n6-c5cc(-c6ccccc6)nc(-c6ccccc6)c5)ccc43)c2)cc1]

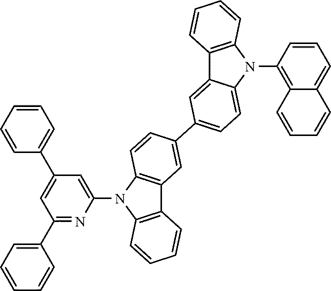 [SMILES: c1ccc(-c2cc(-c3ccccc3)nc(-n3c4ccccc4c4cc(-c5ccc6c(c5)c5ccccc5n6-c5cccc6ccccc56)ccc43)c2)cc1]

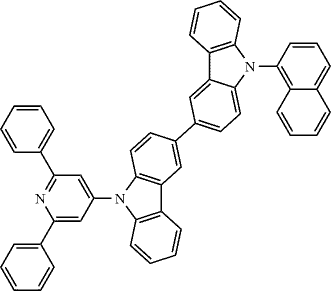 [SMILES: c1ccc(-c2cc(-n3c4ccccc4c4cc(-c5ccc6c(c5)c5ccccc5n6-c5cccc6ccccc56)ccc43)cc(-c3ccccc3)n2)cc1]

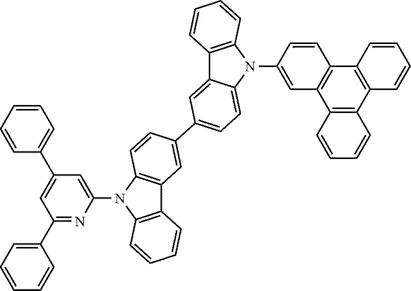 [SMILES: c1ccc(-c2cc(-c3ccccc3)nc(-n3c4ccccc4c4cc(-c5ccc6c(c5)c5ccccc5n6-c5ccc6c7ccccc7c7ccccc7c6c5)ccc43)c2)cc1]

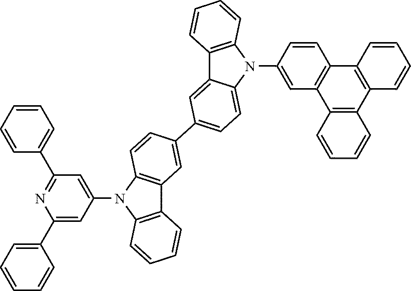 [SMILES: c1ccc(-c2cc(-n3c4ccccc4c4cc(-c5ccc6c(c5)c5ccccc5n6-c5ccc6c7ccccc7c7ccccc7c6c5)ccc43)cc(-c3ccccc3)n2)cc1]

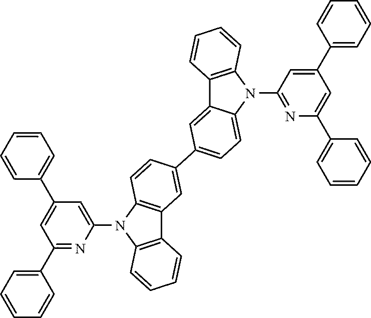 [SMILES: c1ccc(-c2cc(-c3ccccc3)nc(-n3c4ccccc4c4cc(-c5ccc6c(c5)c5ccccc5n6-c5cc(-c6ccccc6)cc(-c6ccccc6)n5)ccc43)c2)cc1]

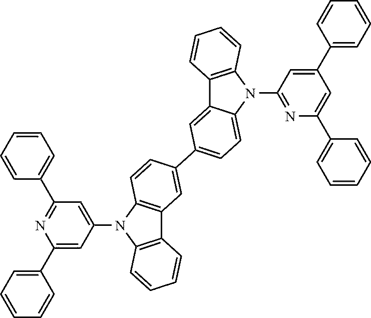 [SMILES: c1ccc(-c2cc(-c3ccccc3)nc(-n3c4ccccc4c4cc(-c5ccc6c(c5)c5ccccc5n6-c5cc(-c6ccccc6)nc(-c6ccccc6)c5)ccc43)c2)cc1]

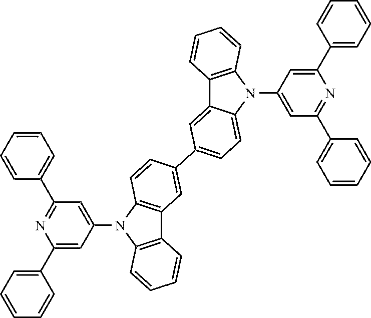 [SMILES: c1ccc(-c2cc(-n3c4ccccc4c4cc(-c5ccc6c(c5)c5ccccc5n6-c5cc(-c6ccccc6)nc(-c6ccccc6)c5)ccc43)cc(-c3ccccc3)n2)cc1]

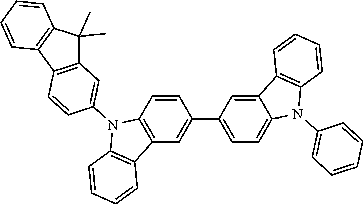 [SMILES: CC1(C)c2ccccc2-c2ccc(-n3c4ccccc4c4cc(-c5ccc6c(c5)c5ccccc5n6-c5ccccc5)ccc43)cc21]

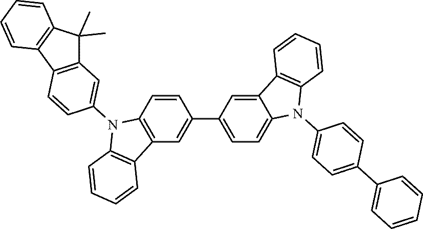 [SMILES: CC1(C)c2ccccc2-c2ccc(-n3c4ccccc4c4cc(-c5ccc6c(c5)c5ccccc5n6-c5ccc(-c6ccccc6)cc5)ccc43)cc21]

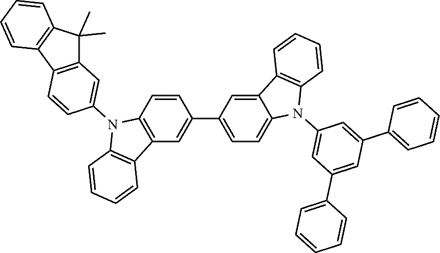 [SMILES: CC1(C)c2ccccc2-c2ccc(-n3c4ccccc4c4cc(-c5ccc6c(c5)c5ccccc5n6-c5cc(-c6ccccc6)cc(-c6ccccc6)c5)ccc43)cc21]

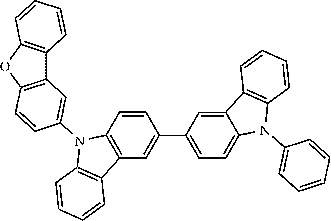 [SMILES: c1ccc(-n2c3ccccc3c3cc(-c4ccc5c(c4)c4ccccc4n5-c4ccc5oc6ccccc6c5c4)ccc32)cc1]

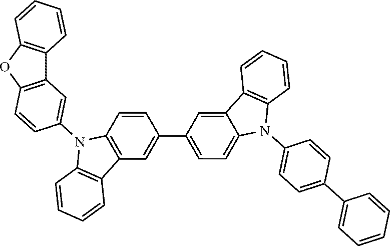 [SMILES: c1ccc(-c2ccc(-n3c4ccccc4c4cc(-c5ccc6c(c5)c5ccccc5n6-c5ccc6oc7ccccc7c6c5)ccc43)cc2)cc1]

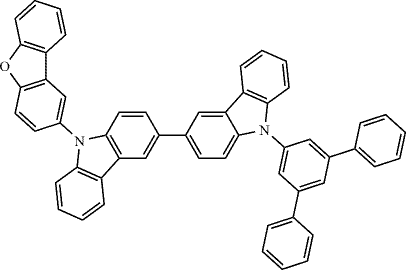 [SMILES: c1ccc(-c2cc(-c3ccccc3)cc(-n3c4ccccc4c4cc(-c5ccc6c(c5)c5ccccc5n6-c5ccc6oc7ccccc7c6c5)ccc43)c2)cc1]

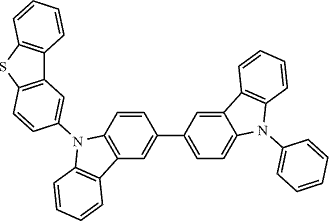 [SMILES: c1ccc(-n2c3ccccc3c3cc(-c4ccc5c(c4)c4ccccc4n5-c4ccc5sc6ccccc6c5c4)ccc32)cc1]

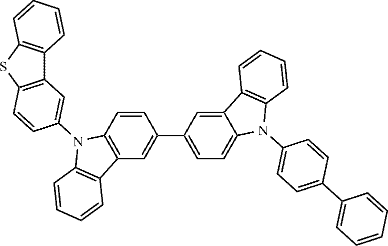 [SMILES: c1ccc(-c2ccc(-n3c4ccccc4c4cc(-c5ccc6c(c5)c5ccccc5n6-c5ccc6sc7ccccc7c6c5)ccc43)cc2)cc1]

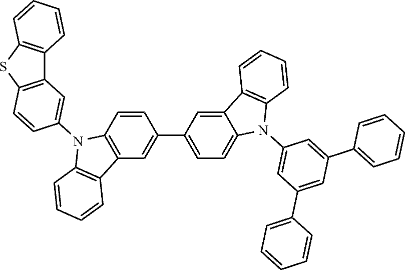 [SMILES: c1ccc(-c2cc(-c3ccccc3)cc(-n3c4ccccc4c4cc(-c5ccc6c(c5)c5ccccc5n6-c5ccc6sc7ccccc7c6c5)ccc43)c2)cc1]

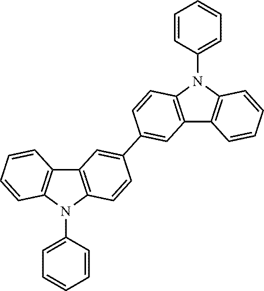 [SMILES: c1ccc(-n2c3ccccc3c3cc(-c4ccc5c(c4)c4ccccc4n5-c4ccccc4)ccc32)cc1]

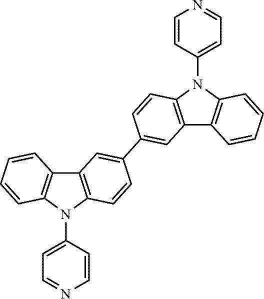 [SMILES: c1ccc2c(c1)c1cc(-c3ccc4c(c3)c3ccccc3n4-c3ccncc3)ccc1n2-c1ccncc1]

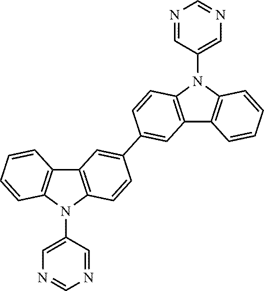 [SMILES: c1ccc2c(c1)c1cc(-c3ccc4c(c3)c3ccccc3n4-c3cncnc3)ccc1n2-c1cncnc1]

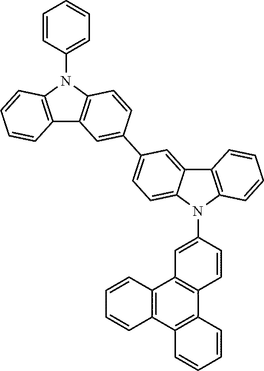 [SMILES: c1ccc(-n2c3ccccc3c3cc(-c4ccc5c(c4)c4ccccc4n5-c4ccc5c6ccccc6c6ccccc6c5c4)ccc32)cc1]

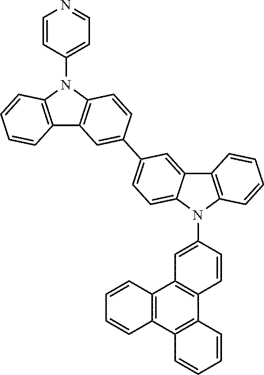 [SMILES: c1ccc2c(c1)c1ccccc1c1cc(-n3c4ccccc4c4cc(-c5ccc6c(c5)c5ccccc5n6-c5ccncc5)ccc43)ccc21]

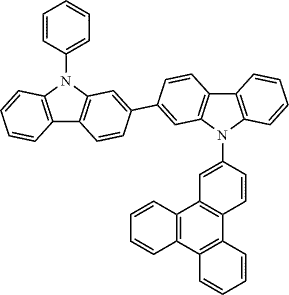 [SMILES: c1ccc(-n2c3ccccc3c3ccc(-c4ccc5c6ccccc6n(-c6ccc7c8ccccc8c8ccccc8c7c6)c5c4)cc32)cc1]

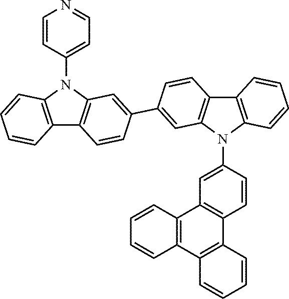 [SMILES: c1ccc2c(c1)c1ccccc1c1cc(-n3c4ccccc4c4ccc(-c5ccc6c7ccccc7n(-c7ccncc7)c6c5)cc43)ccc21]

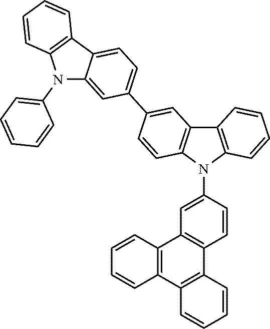 [SMILES: c1ccc(-n2c3ccccc3c3ccc(-c4ccc5c(c4)c4ccccc4n5-c4ccc5c6ccccc6c6ccccc6c5c4)cc32)cc1]

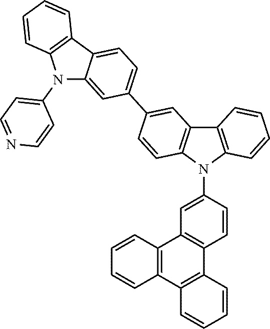 [SMILES: c1ccc2c(c1)c1ccccc1c1cc(-n3c4ccccc4c4cc(-c5ccc6c7ccccc7n(-c7ccncc7)c6c5)ccc43)ccc21]

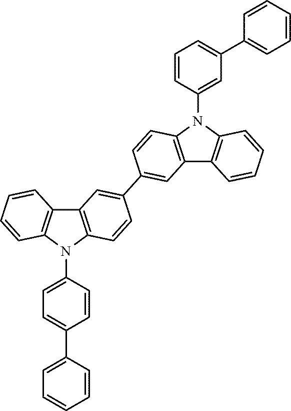 [SMILES: c1ccc(-c2ccc(-n3c4ccccc4c4cc(-c5ccc6c(c5)c5ccccc5n6-c5cccc(-c6ccccc6)c5)ccc43)cc2)cc1]

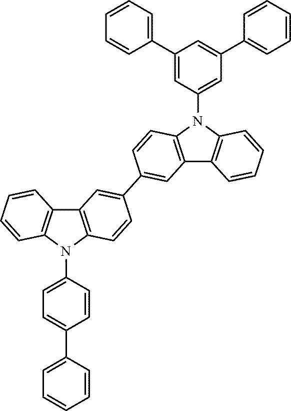 [SMILES: c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1]

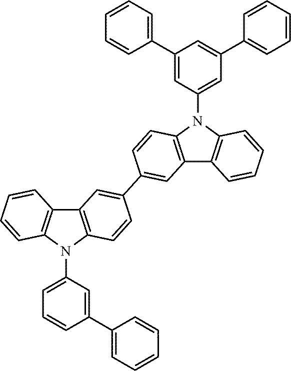 [SMILES: c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1]

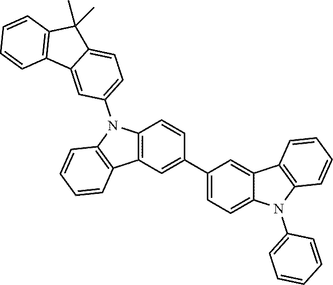 [SMILES: CC1(C)c2ccccc2-c2cc(-n3c4ccccc4c4cc(-c5ccc6c(c5)c5ccccc5n6-c5ccccc5)ccc43)ccc21]

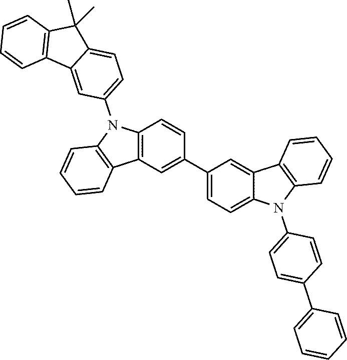 [SMILES: CC1(C)c2ccccc2-c2cc(-n3c4ccccc4c4cc(-c5ccc6c(c5)c5ccccc5n6-c5ccc(-c6ccccc6)cc5)ccc43)ccc21]

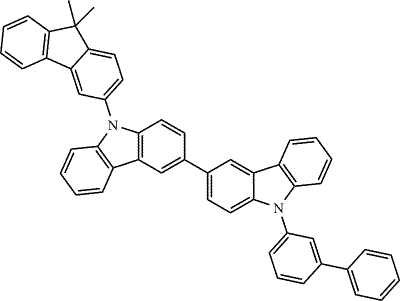 [SMILES: CC1(C)c2ccccc2-c2cc(-n3c4ccccc4c4cc(-c5ccc6c(c5)c5ccccc5n6-c5cccc(-c6ccccc6)c5)ccc43)ccc21]

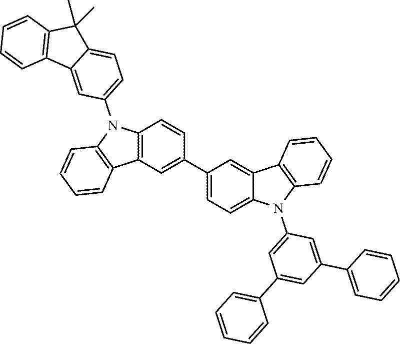 [SMILES: CC1(C)c2ccccc2-c2cc(-n3c4ccccc4c4cc(-c5ccc6c(c5)c5ccccc5n6-c5cc(-c6ccccc6)cc(-c6ccccc6)c5)ccc43)ccc21]

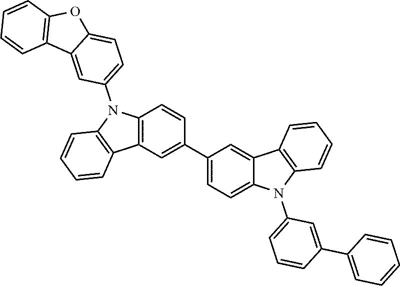 [SMILES: c1ccc(-c2cccc(-n3c4ccccc4c4cc(-c5ccc6c(c5)c5ccccc5n6-c5ccc6oc7ccccc7c6c5)ccc43)c2)cc1]

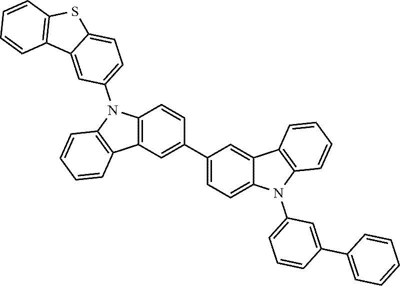 [SMILES: c1ccc(-c2cccc(-n3c4ccccc4c4cc(-c5ccc6c(c5)c5ccccc5n6-c5ccc6sc7ccccc7c6c5)ccc43)c2)cc1]

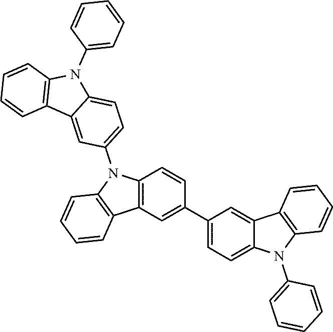 [SMILES: c1ccc(-n2c3ccccc3c3cc(-c4ccc5c(c4)c4ccccc4n5-c4ccc5c(c4)c4ccccc4n5-c4ccccc4)ccc32)cc1]

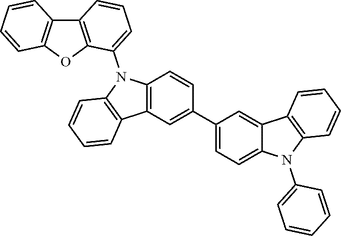 [SMILES: c1ccc(-n2c3ccccc3c3cc(-c4ccc5c(c4)c4ccccc4n5-c4cccc5c4oc4ccccc45)ccc32)cc1]

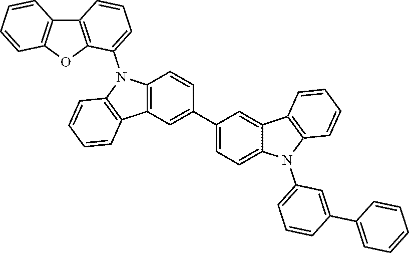 [SMILES: c1ccc(-c2cccc(-n3c4ccccc4c4cc(-c5ccc6c(c5)c5ccccc5n6-c5cccc6c5oc5ccccc56)ccc43)c2)cc1]

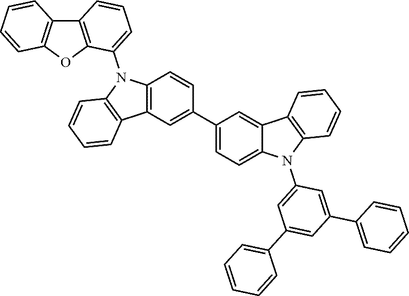 [SMILES: c1ccc(-c2cc(-c3ccccc3)cc(-n3c4ccccc4c4cc(-c5ccc6c(c5)c5ccccc5n6-c5cccc6c5oc5ccccc56)ccc43)c2)cc1]

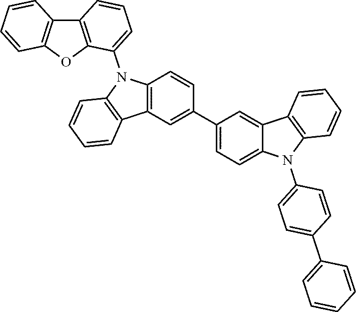 [SMILES: c1ccc(-c2ccc(-n3c4ccccc4c4cc(-c5ccc6c(c5)c5ccccc5n6-c5cccc6c5oc5ccccc56)ccc43)cc2)cc1]

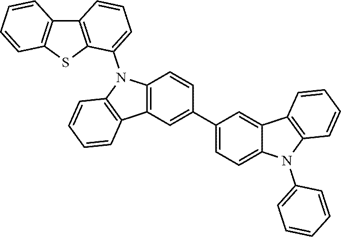 [SMILES: c1ccc(-n2c3ccccc3c3cc(-c4ccc5c(c4)c4ccccc4n5-c4cccc5c4sc4ccccc45)ccc32)cc1]

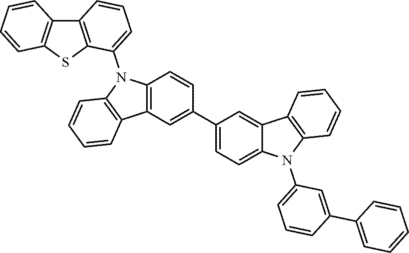 [SMILES: c1ccc(-c2cccc(-n3c4ccccc4c4cc(-c5ccc6c(c5)c5ccccc5n6-c5cccc6c5sc5ccccc56)ccc43)c2)cc1]

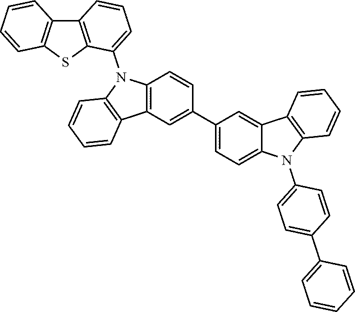 [SMILES: c1ccc(-c2ccc(-n3c4ccccc4c4cc(-c5ccc6c(c5)c5ccccc5n6-c5cccc6c5sc5ccccc56)ccc43)cc2)cc1]

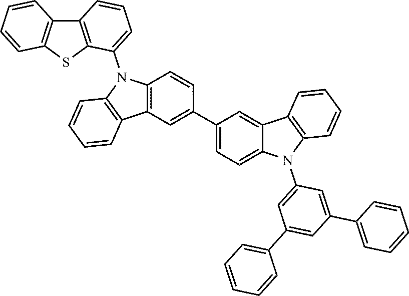 [SMILES: c1ccc(-c2cc(-c3ccccc3)cc(-n3c4ccccc4c4cc(-c5ccc6c(c5)c5ccccc5n6-c5cccc6c5sc5ccccc56)ccc43)c2)cc1]

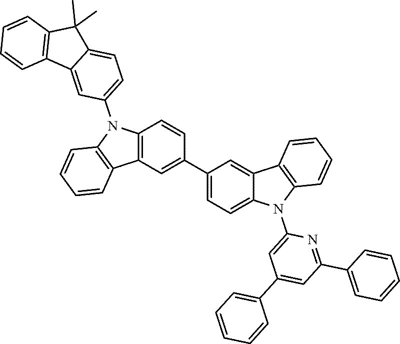 [SMILES: CC1(C)c2ccccc2-c2cc(-n3c4ccccc4c4cc(-c5ccc6c(c5)c5ccccc5n6-c5cc(-c6ccccc6)cc(-c6ccccc6)n5)ccc43)ccc21]

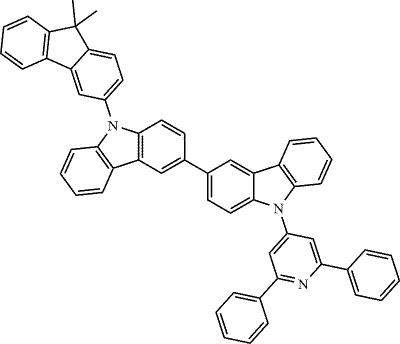 [SMILES: CC1(C)c2ccccc2-c2cc(-n3c4ccccc4c4cc(-c5ccc6c(c5)c5ccccc5n6-c5cc(-c6ccccc6)nc(-c6ccccc6)c5)ccc43)ccc21]

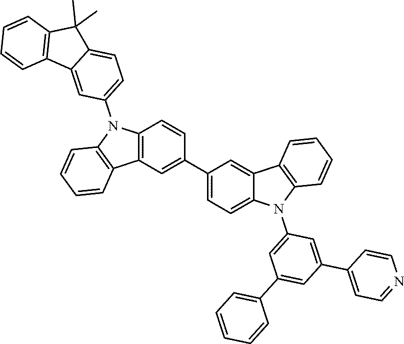 [SMILES: CC1(C)c2ccccc2-c2cc(-n3c4ccccc4c4cc(-c5ccc6c(c5)c5ccccc5n6-c5cc(-c6ccccc6)cc(-c6ccncc6)c5)ccc43)ccc21]

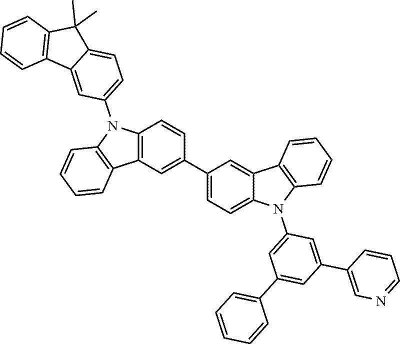 [SMILES: CC1(C)c2ccccc2-c2cc(-n3c4ccccc4c4cc(-c5ccc6c(c5)c5ccccc5n6-c5cc(-c6ccccc6)cc(-c6cccnc6)c5)ccc43)ccc21]

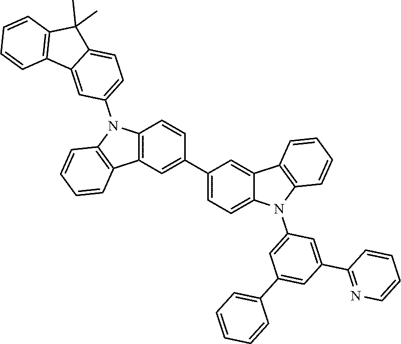 [SMILES: CC1(C)c2ccccc2-c2cc(-n3c4ccccc4c4cc(-c5ccc6c(c5)c5ccccc5n6-c5cc(-c6ccccc6)cc(-c6ccccn6)c5)ccc43)ccc21]

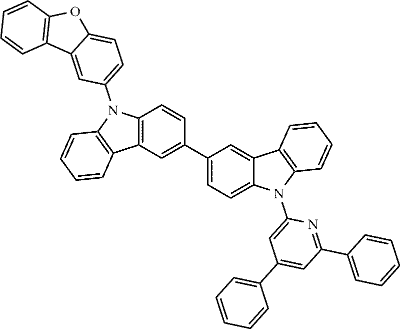 [SMILES: c1ccc(-c2cc(-c3ccccc3)nc(-n3c4ccccc4c4cc(-c5ccc6c(c5)c5ccccc5n6-c5ccc6oc7ccccc7c6c5)ccc43)c2)cc1]

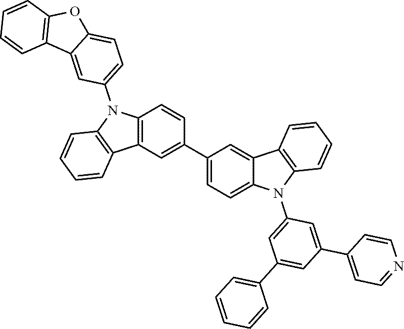 [SMILES: c1ccc(-c2cc(-c3ccncc3)cc(-n3c4ccccc4c4cc(-c5ccc6c(c5)c5ccccc5n6-c5ccc6oc7ccccc7c6c5)ccc43)c2)cc1]

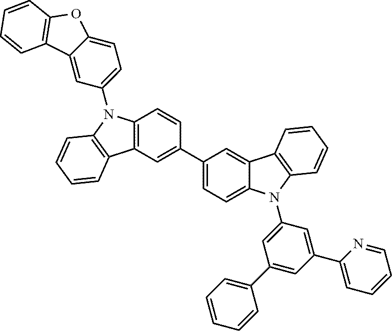 [SMILES: c1ccc(-c2cc(-c3ccccn3)cc(-n3c4ccccc4c4cc(-c5ccc6c(c5)c5ccccc5n6-c5ccc6oc7ccccc7c6c5)ccc43)c2)cc1]

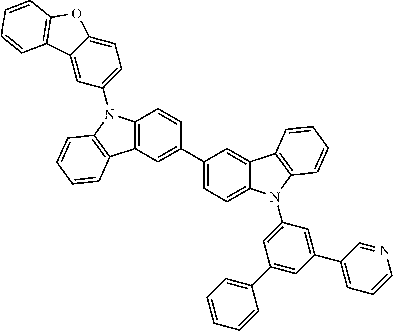 [SMILES: c1ccc(-c2cc(-c3cccnc3)cc(-n3c4ccccc4c4cc(-c5ccc6c(c5)c5ccccc5n6-c5ccc6oc7ccccc7c6c5)ccc43)c2)cc1]

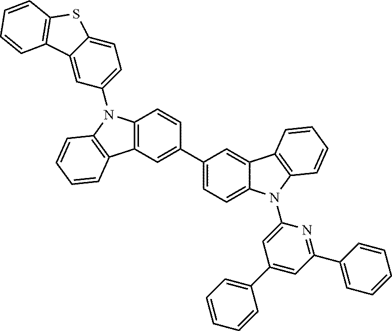 [SMILES: c1ccc(-c2cc(-c3ccccc3)nc(-n3c4ccccc4c4cc(-c5ccc6c(c5)c5ccccc5n6-c5ccc6sc7ccccc7c6c5)ccc43)c2)cc1]

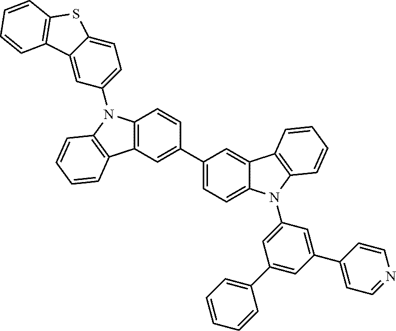 [SMILES: c1ccc(-c2cc(-c3ccncc3)cc(-n3c4ccccc4c4cc(-c5ccc6c(c5)c5ccccc5n6-c5ccc6sc7ccccc7c6c5)ccc43)c2)cc1]

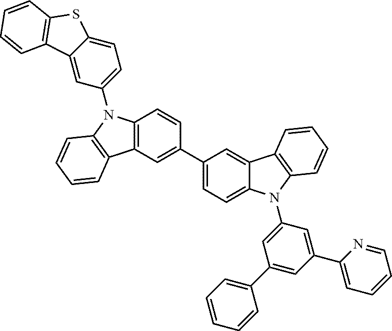 [SMILES: c1ccc(-c2cc(-c3ccccn3)cc(-n3c4ccccc4c4cc(-c5ccc6c(c5)c5ccccc5n6-c5ccc6sc7ccccc7c6c5)ccc43)c2)cc1]

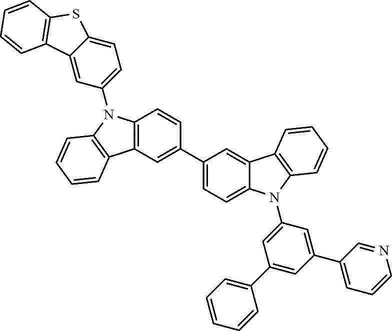 [SMILES: c1ccc(-c2cc(-c3cccnc3)cc(-n3c4ccccc4c4cc(-c5ccc6c(c5)c5ccccc5n6-c5ccc6sc7ccccc7c6c5)ccc43)c2)cc1]

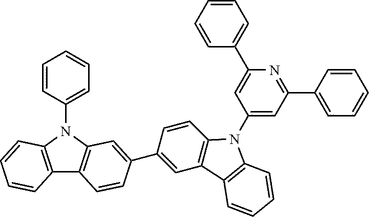 [SMILES: c1ccc(-c2cc(-n3c4ccccc4c4cc(-c5ccc6c7ccccc7n(-c7ccccc7)c6c5)ccc43)cc(-c3ccccc3)n2)cc1]

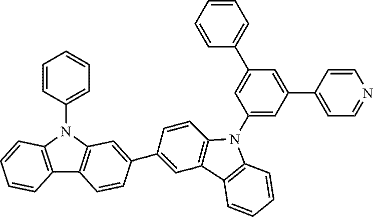 [SMILES: c1ccc(-c2cc(-c3ccncc3)cc(-n3c4ccccc4c4cc(-c5ccc6c7ccccc7n(-c7ccccc7)c6c5)ccc43)c2)cc1]

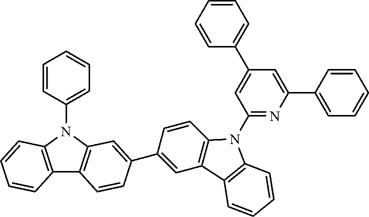 [SMILES: c1ccc(-c2cc(-c3ccccc3)nc(-n3c4ccccc4c4cc(-c5ccc6c7ccccc7n(-c7ccccc7)c6c5)ccc43)c2)cc1]

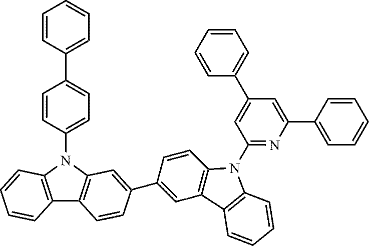 [SMILES: c1ccc(-c2ccc(-n3c4ccccc4c4ccc(-c5ccc6c(c5)c5ccccc5n6-c5cc(-c6ccccc6)cc(-c6ccccc6)n5)cc43)cc2)cc1]

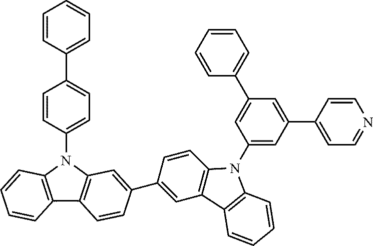 [SMILES: c1ccc(-c2ccc(-n3c4ccccc4c4ccc(-c5ccc6c(c5)c5ccccc5n6-c5cc(-c6ccccc6)cc(-c6ccncc6)c5)cc43)cc2)cc1]

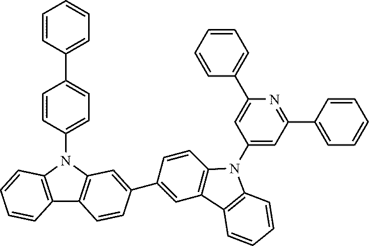 [SMILES: c1ccc(-c2ccc(-n3c4ccccc4c4ccc(-c5ccc6c(c5)c5ccccc5n6-c5cc(-c6ccccc6)nc(-c6ccccc6)c5)cc43)cc2)cc1]

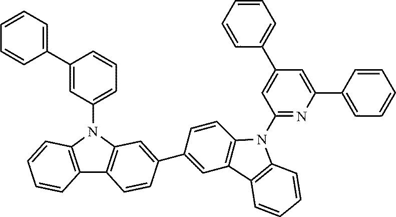 [SMILES: c1ccc(-c2cccc(-n3c4ccccc4c4ccc(-c5ccc6c(c5)c5ccccc5n6-c5cc(-c6ccccc6)cc(-c6ccccc6)n5)cc43)c2)cc1]

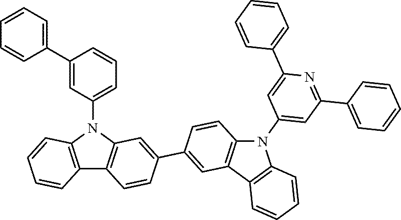 [SMILES: c1ccc(-c2cccc(-n3c4ccccc4c4ccc(-c5ccc6c(c5)c5ccccc5n6-c5cc(-c6ccccc6)nc(-c6ccccc6)c5)cc43)c2)cc1]

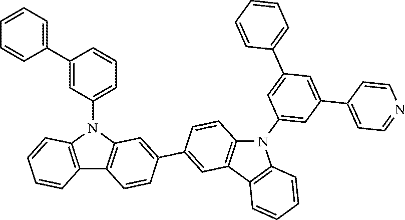 [SMILES: c1ccc(-c2cccc(-n3c4ccccc4c4ccc(-c5ccc6c(c5)c5ccccc5n6-c5cc(-c6ccccc6)cc(-c6ccncc6)c5)cc43)c2)cc1]

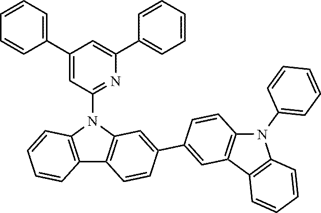 [SMILES: c1ccc(-c2cc(-c3ccccc3)nc(-n3c4ccccc4c4ccc(-c5ccc6c(c5)c5ccccc5n6-c5ccccc5)cc43)c2)cc1]

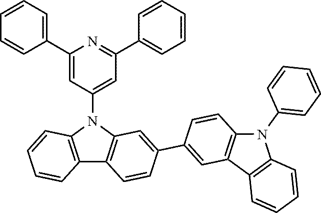 [SMILES: c1ccc(-c2cc(-n3c4ccccc4c4ccc(-c5ccc6c(c5)c5ccccc5n6-c5ccccc5)cc43)cc(-c3ccccc3)n2)cc1]

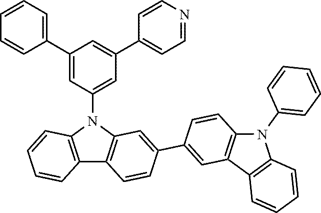 [SMILES: c1ccc(-c2cc(-c3ccncc3)cc(-n3c4ccccc4c4ccc(-c5ccc6c(c5)c5ccccc5n6-c5ccccc5)cc43)c2)cc1]

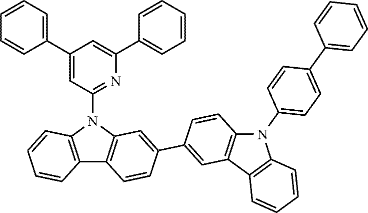 [SMILES: c1ccc(-c2ccc(-n3c4ccccc4c4cc(-c5ccc6c7ccccc7n(-c7cc(-c8ccccc8)cc(-c8ccccc8)n7)c6c5)ccc43)cc2)cc1]

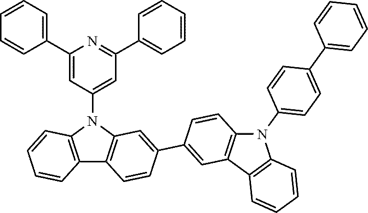 [SMILES: c1ccc(-c2ccc(-n3c4ccccc4c4cc(-c5ccc6c7ccccc7n(-c7cc(-c8ccccc8)nc(-c8ccccc8)c7)c6c5)ccc43)cc2)cc1]

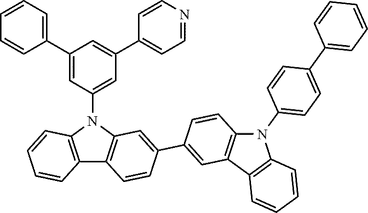 [SMILES: c1ccc(-c2ccc(-n3c4ccccc4c4cc(-c5ccc6c7ccccc7n(-c7cc(-c8ccccc8)cc(-c8ccncc8)c7)c6c5)ccc43)cc2)cc1]

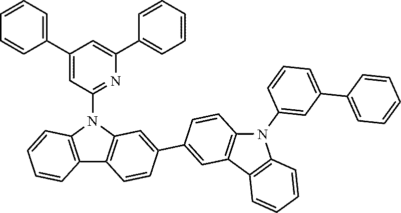 [SMILES: c1ccc(-c2cccc(-n3c4ccccc4c4cc(-c5ccc6c7ccccc7n(-c7cc(-c8ccccc8)cc(-c8ccccc8)n7)c6c5)ccc43)c2)cc1]

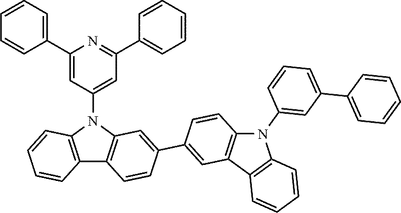 [SMILES: c1ccc(-c2cccc(-n3c4ccccc4c4cc(-c5ccc6c7ccccc7n(-c7cc(-c8ccccc8)nc(-c8ccccc8)c7)c6c5)ccc43)c2)cc1]

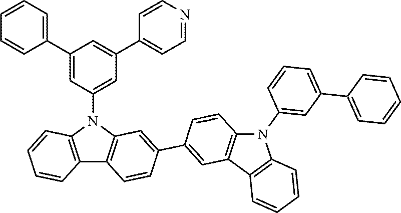 [SMILES: c1ccc(-c2cccc(-n3c4ccccc4c4cc(-c5ccc6c7ccccc7n(-c7cc(-c8ccccc8)cc(-c8ccncc8)c7)c6c5)ccc43)c2)cc1]

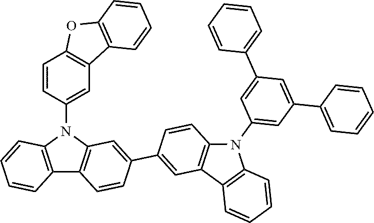 [SMILES: c1ccc(-c2cc(-c3ccccc3)cc(-n3c4ccccc4c4cc(-c5ccc6c7ccccc7n(-c7ccc8oc9ccccc9c8c7)c6c5)ccc43)c2)cc1]

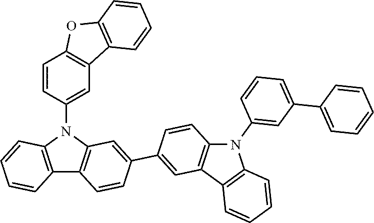 [SMILES: c1ccc(-c2cccc(-n3c4ccccc4c4cc(-c5ccc6c7ccccc7n(-c7ccc8oc9ccccc9c8c7)c6c5)ccc43)c2)cc1]

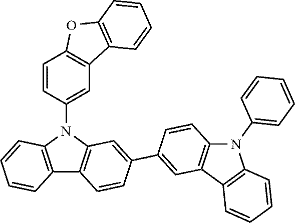 [SMILES: c1ccc(-n2c3ccccc3c3cc(-c4ccc5c6ccccc6n(-c6ccc7oc8ccccc8c7c6)c5c4)ccc32)cc1]

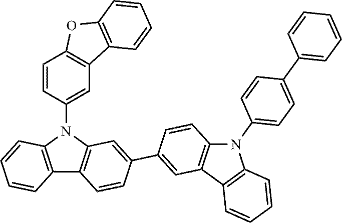 [SMILES: c1ccc(-c2ccc(-n3c4ccccc4c4cc(-c5ccc6c7ccccc7n(-c7ccc8oc9ccccc9c8c7)c6c5)ccc43)cc2)cc1]

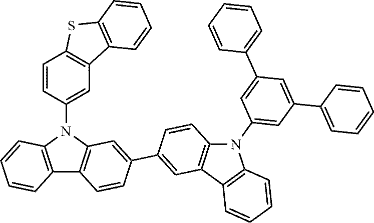 [SMILES: c1ccc(-c2cc(-c3ccccc3)cc(-n3c4ccccc4c4cc(-c5ccc6c7ccccc7n(-c7ccc8sc9ccccc9c8c7)c6c5)ccc43)c2)cc1]

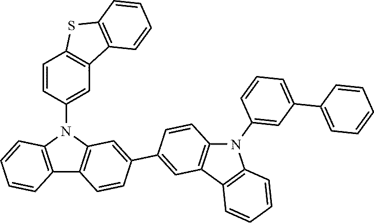 [SMILES: c1ccc(-c2cccc(-n3c4ccccc4c4cc(-c5ccc6c7ccccc7n(-c7ccc8sc9ccccc9c8c7)c6c5)ccc43)c2)cc1]

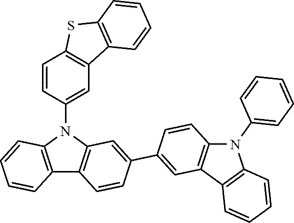 [SMILES: c1ccc(-n2c3ccccc3c3cc(-c4ccc5c6ccccc6n(-c6ccc7sc8ccccc8c7c6)c5c4)ccc32)cc1]

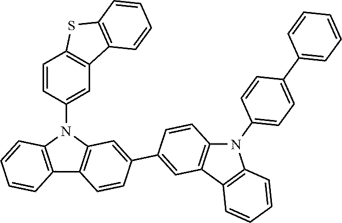 [SMILES: c1ccc(-c2ccc(-n3c4ccccc4c4cc(-c5ccc6c7ccccc7n(-c7ccc8sc9ccccc9c8c7)c6c5)ccc43)cc2)cc1]

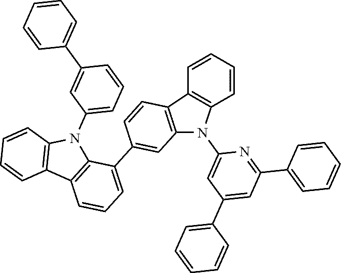 [SMILES: c1ccc(-c2cccc(-n3c4ccccc4c4cccc(-c5ccc6c7ccccc7n(-c7cc(-c8ccccc8)cc(-c8ccccc8)n7)c6c5)c43)c2)cc1]

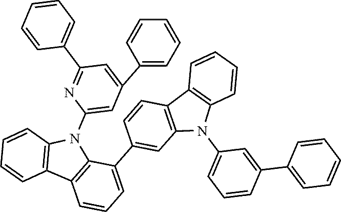 [SMILES: c1ccc(-c2cccc(-n3c4ccccc4c4ccc(-c5cccc6c7ccccc7n(-c7cc(-c8ccccc8)cc(-c8ccccc8)n7)c56)cc43)c2)cc1]

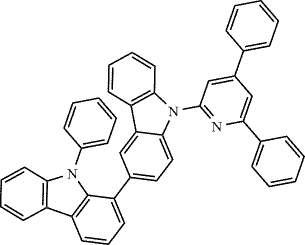 [SMILES: c1ccc(-c2cc(-c3ccccc3)nc(-n3c4ccccc4c4cc(-c5cccc6c7ccccc7n(-c7ccccc7)c56)ccc43)c2)cc1]

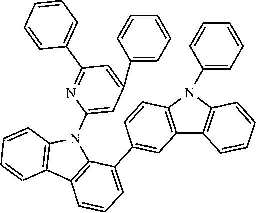 [SMILES: c1ccc(-c2cc(-c3ccccc3)nc(-n3c4ccccc4c4cccc(-c5ccc6c(c5)c5ccccc5n6-c5ccccc5)c43)c2)cc1]

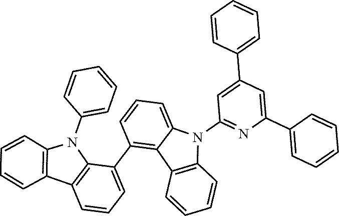 [SMILES: c1ccc(-c2cc(-c3ccccc3)nc(-n3c4ccccc4c4c(-c5cccc6c7ccccc7n(-c7ccccc7)c56)cccc43)c2)cc1]

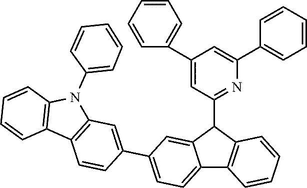 [SMILES: c1ccc(-c2cc(-c3ccccc3)nc(C3c4ccccc4-c4ccc(-c5ccc6c7ccccc7n(-c7ccccc7)c6c5)cc43)c2)cc1]

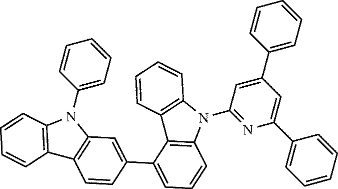 [SMILES: c1ccc(-c2cc(-c3ccccc3)nc(-n3c4ccccc4c4c(-c5ccc6c7ccccc7n(-c7ccccc7)c6c5)cccc43)c2)cc1]

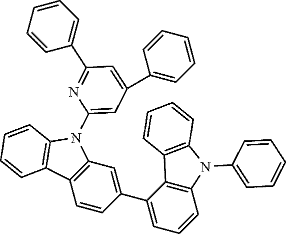 [SMILES: c1ccc(-c2cc(-c3ccccc3)nc(-n3c4ccccc4c4ccc(-c5cccc6c5c5ccccc5n6-c5ccccc5)cc43)c2)cc1]

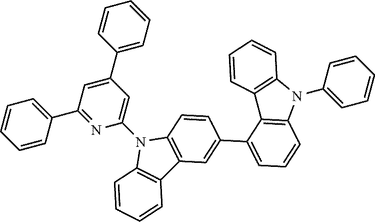 [SMILES: c1ccc(-c2cc(-c3ccccc3)nc(-n3c4ccccc4c4cc(-c5cccc6c5c5ccccc5n6-c5ccccc5)ccc43)c2)cc1]

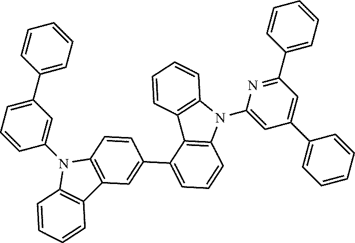 [SMILES: c1ccc(-c2cccc(-n3c4ccccc4c4cc(-c5cccc6c5c5ccccc5n6-c5cc(-c6ccccc6)cc(-c6ccccc6)n5)ccc43)c2)cc1]

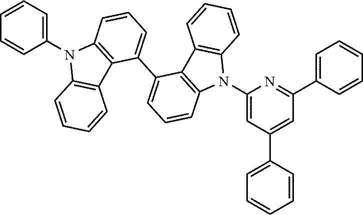 [SMILES: c1ccc(-c2cc(-c3ccccc3)nc(-n3c4ccccc4c4c(-c5cccc6c5c5ccccc5n6-c5ccccc5)cccc43)c2)cc1]

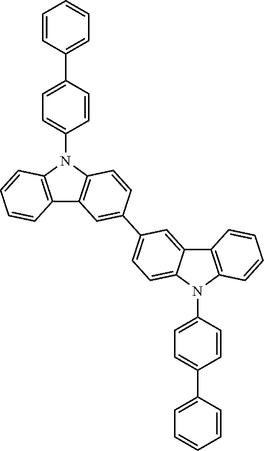 [SMILES: c1ccc(-c2ccc(-n3c4ccccc4c4cc(-c5ccc6c(c5)c5ccccc5n6-c5ccc(-c6ccccc6)cc5)ccc43)cc2)cc1]

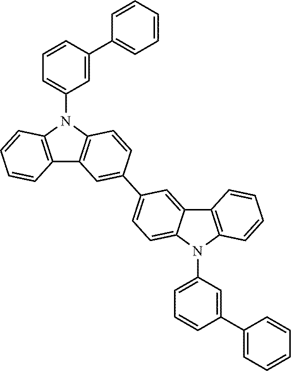 [SMILES: c1ccc(-c2cccc(-n3c4ccccc4c4cc(-c5ccc6c(c5)c5ccccc5n6-c5cccc(-c6ccccc6)c5)ccc43)c2)cc1]

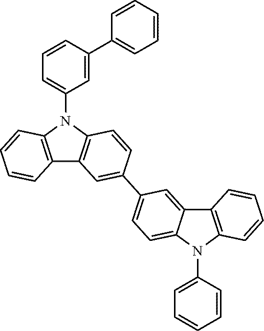 [SMILES: c1ccc(-c2cccc(-n3c4ccccc4c4cc(-c5ccc6c(c5)c5ccccc5n6-c5ccccc5)ccc43)c2)cc1]

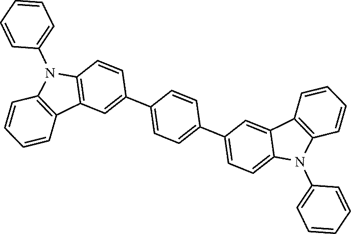 [SMILES: c1ccc(-n2c3ccccc3c3cc(-c4ccc(-c5ccc6c(c5)c5ccccc5n6-c5ccccc5)cc4)ccc32)cc1]

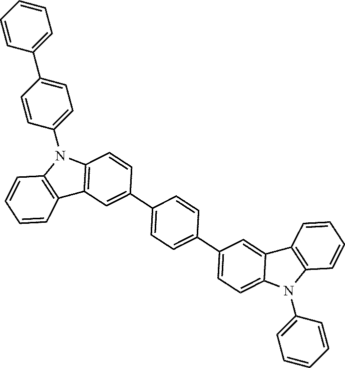 [SMILES: c1ccc(-c2ccc(-n3c4ccccc4c4cc(-c5ccc(-c6ccc7c(c6)c6ccccc6n7-c6ccccc6)cc5)ccc43)cc2)cc1]

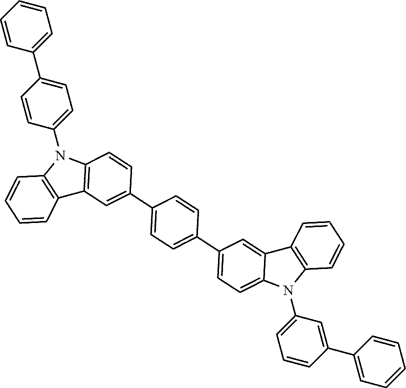 [SMILES: c1ccc(-c2ccc(-n3c4ccccc4c4cc(-c5ccc(-c6ccc7c(c6)c6ccccc6n7-c6cccc(-c7ccccc7)c6)cc5)ccc43)cc2)cc1]

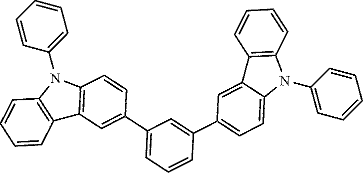 [SMILES: c1ccc(-n2c3ccccc3c3cc(-c4cccc(-c5ccc6c(c5)c5ccccc5n6-c5ccccc5)c4)ccc32)cc1]

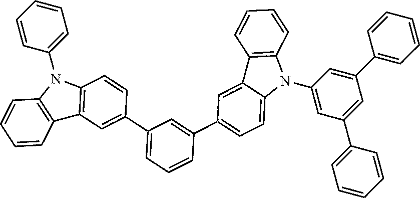 [SMILES: c1ccc(-c2cc(-c3ccccc3)cc(-n3c4ccccc4c4cc(-c5cccc(-c6ccc7c(c6)c6ccccc6n7-c6ccccc6)c5)ccc43)c2)cc1]

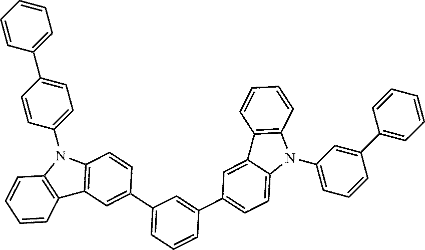 [SMILES: c1ccc(-c2ccc(-n3c4ccccc4c4cc(-c5cccc(-c6ccc7c(c6)c6ccccc6n7-c6cccc(-c7ccccc7)c6)c5)ccc43)cc2)cc1]

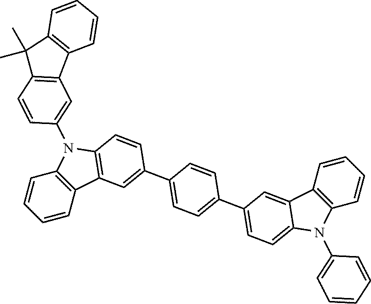 [SMILES: CC1(C)c2ccccc2-c2cc(-n3c4ccccc4c4cc(-c5ccc(-c6ccc7c(c6)c6ccccc6n7-c6ccccc6)cc5)ccc43)ccc21]

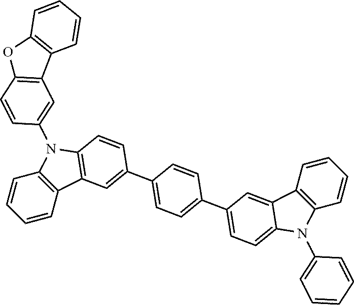 [SMILES: c1ccc(-n2c3ccccc3c3cc(-c4ccc(-c5ccc6c(c5)c5ccccc5n6-c5ccc6oc7ccccc7c6c5)cc4)ccc32)cc1]

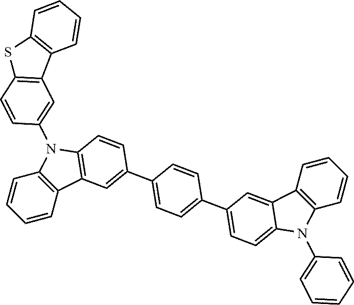 [SMILES: c1ccc(-n2c3ccccc3c3cc(-c4ccc(-c5ccc6c(c5)c5ccccc5n6-c5ccc6sc7ccccc7c6c5)cc4)ccc32)cc1]

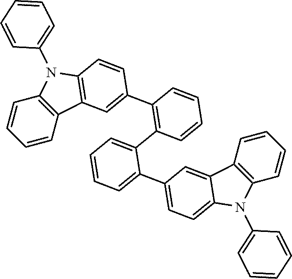 [SMILES: c1ccc(-n2c3ccccc3c3cc(-c4ccccc4-c4ccccc4-c4ccc5c(c4)c4ccccc4n5-c4ccccc4)ccc32)cc1]

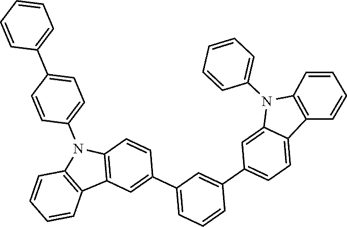 [SMILES: c1ccc(-c2ccc(-n3c4ccccc4c4cc(-c5cccc(-c6ccc7c8ccccc8n(-c8ccccc8)c7c6)c5)ccc43)cc2)cc1]

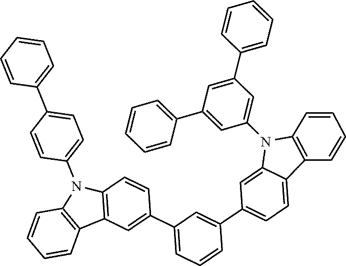 [SMILES: c1ccc(-c2ccc(-n3c4ccccc4c4cc(-c5cccc(-c6ccc7c8ccccc8n(-c8cc(-c9ccccc9)cc(-c9ccccc9)c8)c7c6)c5)ccc43)cc2)cc1]

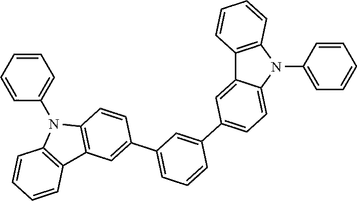 [SMILES: c1ccc(-n2c3ccccc3c3cc(-c4cccc(-c5ccc6c(c5)c5ccccc5n6-c5ccccc5)c4)ccc32)cc1]

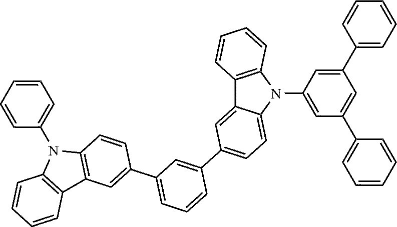 [SMILES: c1ccc(-c2cc(-c3ccccc3)cc(-n3c4ccccc4c4cc(-c5cccc(-c6ccc7c(c6)c6ccccc6n7-c6ccccc6)c5)ccc43)c2)cc1]

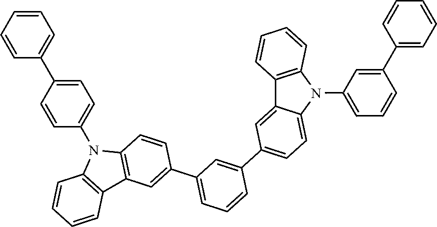 [SMILES: c1ccc(-c2ccc(-n3c4ccccc4c4cc(-c5cccc(-c6ccc7c(c6)c6ccccc6n7-c6cccc(-c7ccccc7)c6)c5)ccc43)cc2)cc1]

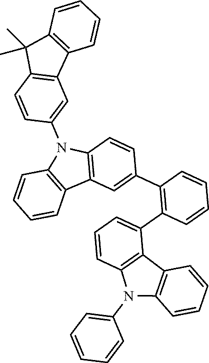 [SMILES: CC1(C)c2ccccc2-c2cc(-n3c4ccccc4c4cc(-c5ccccc5-c5cccc6c5c5ccccc5n6-c5ccccc5)ccc43)ccc21]

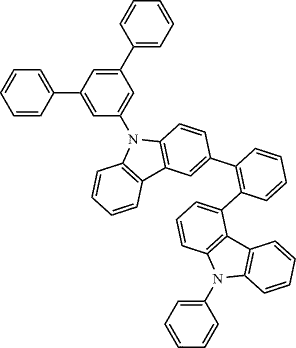 [SMILES: c1ccc(-c2cc(-c3ccccc3)cc(-n3c4ccccc4c4cc(-c5ccccc5-c5cccc6c5c5ccccc5n6-c5ccccc5)ccc43)c2)cc1]

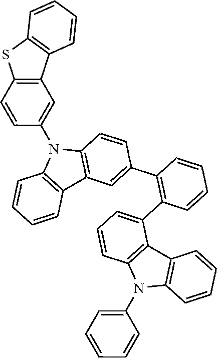 [SMILES: c1ccc(-n2c3ccccc3c3c(-c4ccccc4-c4ccc5c(c4)c4ccccc4n5-c4ccc5sc6ccccc6c5c4)cccc32)cc1]

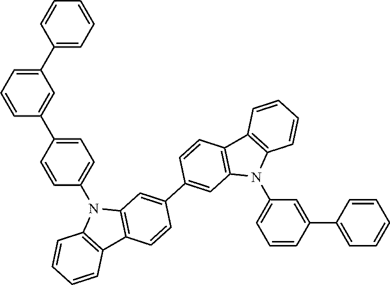 [SMILES: c1ccc(-c2cccc(-c3ccc(-n4c5ccccc5c5ccc(-c6ccc7c8ccccc8n(-c8cccc(-c9ccccc9)c8)c7c6)cc54)cc3)c2)cc1]

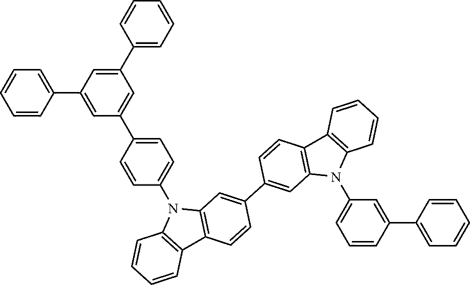 [SMILES: c1ccc(-c2cc(-c3ccccc3)cc(-c3ccc(-n4c5ccccc5c5ccc(-c6ccc7c8ccccc8n(-c8cccc(-c9ccccc9)c8)c7c6)cc54)cc3)c2)cc1]

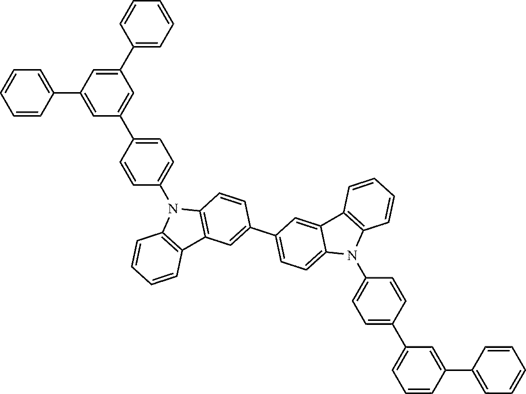 [SMILES: c1ccc(-c2cccc(-c3ccc(-n4c5ccccc5c5cc(-c6ccc7c(c6)c6ccccc6n7-c6ccc(-c7cc(-c8ccccc8)cc(-c8ccccc8)c7)cc6)ccc54)cc3)c2)cc1]

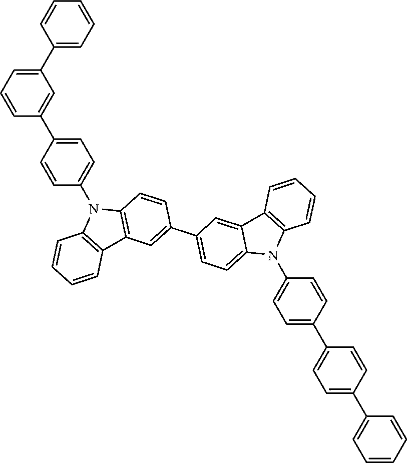 [SMILES: c1ccc(-c2ccc(-c3ccc(-n4c5ccccc5c5cc(-c6ccc7c(c6)c6ccccc6n7-c6ccc(-c7cccc(-c8ccccc8)c7)cc6)ccc54)cc3)cc2)cc1]

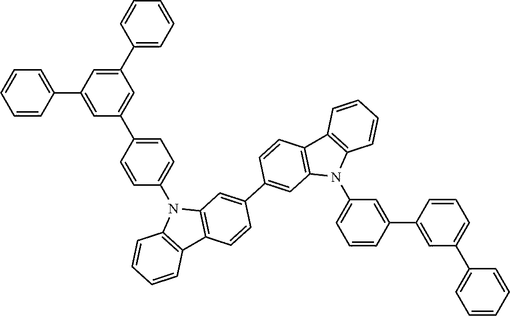 [SMILES: c1ccc(-c2cccc(-c3cccc(-n4c5ccccc5c5ccc(-c6ccc7c8ccccc8n(-c8ccc(-c9cc(-c%10ccccc%10)cc(-c%10ccccc%10)c9)cc8)c7c6)cc54)c3)c2)cc1]

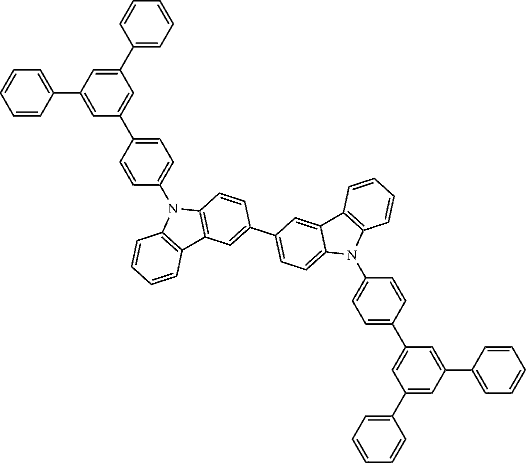 [SMILES: c1ccc(-c2cc(-c3ccccc3)cc(-c3ccc(-n4c5ccccc5c5cc(-c6ccc7c(c6)c6ccccc6n7-c6ccc(-c7cc(-c8ccccc8)cc(-c8ccccc8)c7)cc6)ccc54)cc3)c2)cc1]

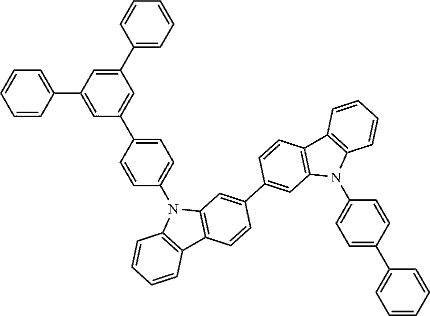 [SMILES: c1ccc(-c2ccc(-n3c4ccccc4c4ccc(-c5ccc6c7ccccc7n(-c7ccc(-c8cc(-c9ccccc9)cc(-c9ccccc9)c8)cc7)c6c5)cc43)cc2)cc1]

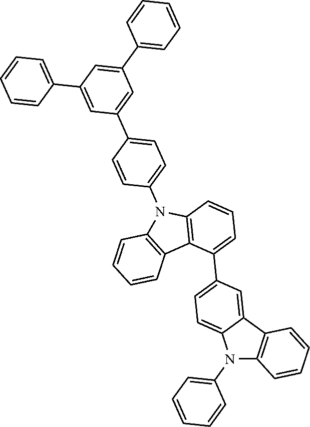 [SMILES: c1ccc(-c2cc(-c3ccccc3)cc(-c3ccc(-n4c5ccccc5c5c(-c6ccc7c(c6)c6ccccc6n7-c6ccccc6)cccc54)cc3)c2)cc1]

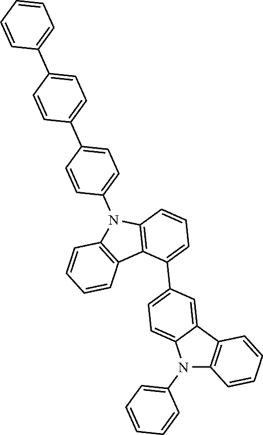 [SMILES: c1ccc(-c2ccc(-c3ccc(-n4c5ccccc5c5c(-c6ccc7c(c6)c6ccccc6n7-c6ccccc6)cccc54)cc3)cc2)cc1]

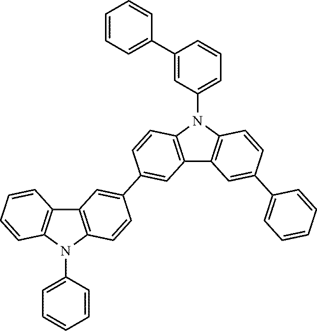 [SMILES: c1ccc(-c2cccc(-n3c4ccc(-c5ccccc5)cc4c4cc(-c5ccc6c(c5)c5ccccc5n6-c5ccccc5)ccc43)c2)cc1]

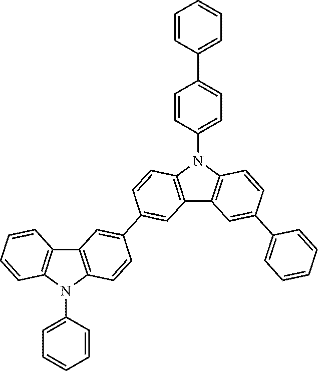 [SMILES: c1ccc(-c2ccc(-n3c4ccc(-c5ccccc5)cc4c4cc(-c5ccc6c(c5)c5ccccc5n6-c5ccccc5)ccc43)cc2)cc1]

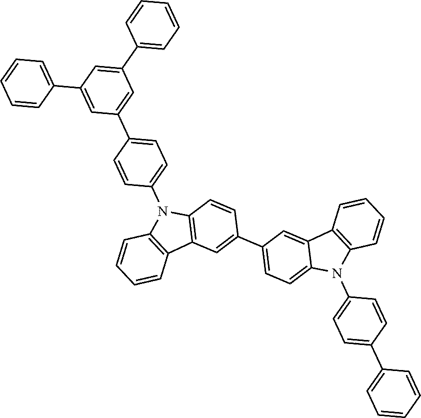 [SMILES: c1ccc(-c2ccc(-n3c4ccccc4c4cc(-c5ccc6c(c5)c5ccccc5n6-c5ccc(-c6cc(-c7ccccc7)cc(-c7ccccc7)c6)cc5)ccc43)cc2)cc1]

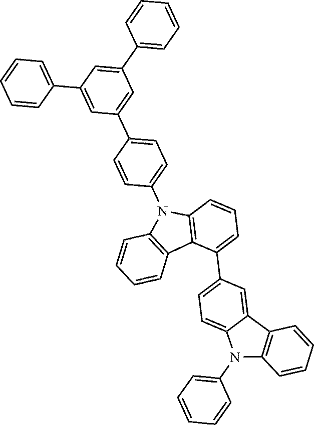 [SMILES: c1ccc(-c2cc(-c3ccccc3)cc(-c3ccc(-n4c5ccccc5c5c(-c6ccc7c(c6)c6ccccc6n7-c6ccccc6)cccc54)cc3)c2)cc1]

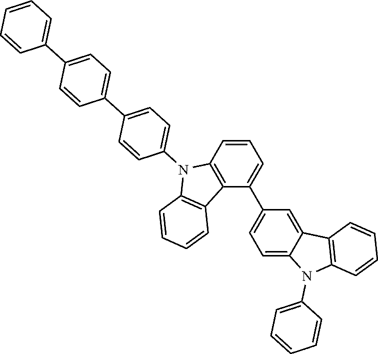 [SMILES: c1ccc(-c2ccc(-c3ccc(-n4c5ccccc5c5c(-c6ccc7c(c6)c6ccccc6n7-c6ccccc6)cccc54)cc3)cc2)cc1]

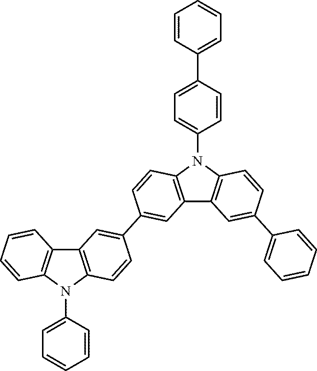 [SMILES: c1ccc(-c2ccc(-n3c4ccc(-c5ccccc5)cc4c4cc(-c5ccc6c(c5)c5ccccc5n6-c5ccccc5)ccc43)cc2)cc1]

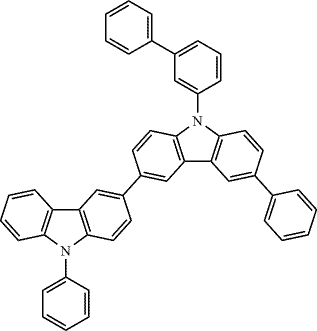 [SMILES: c1ccc(-c2cccc(-n3c4ccc(-c5ccccc5)cc4c4cc(-c5ccc6c(c5)c5ccccc5n6-c5ccccc5)ccc43)c2)cc1]

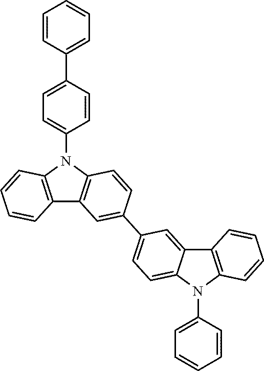 [SMILES: c1ccc(-c2ccc(-n3c4ccccc4c4cc(-c5ccc6c(c5)c5ccccc5n6-c5ccccc5)ccc43)cc2)cc1]

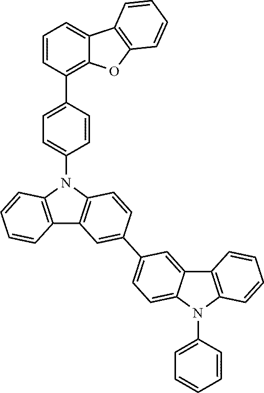 [SMILES: c1ccc(-n2c3ccccc3c3cc(-c4ccc5c(c4)c4ccccc4n5-c4ccc(-c5cccc6c5oc5ccccc56)cc4)ccc32)cc1]

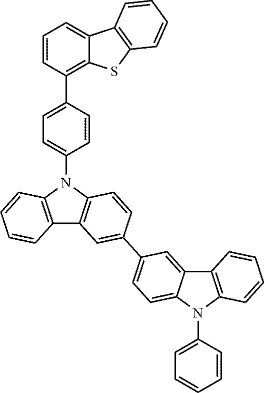 [SMILES: c1ccc(-n2c3ccccc3c3cc(-c4ccc5c(c4)c4ccccc4n5-c4ccc(-c5cccc6c5sc5ccccc56)cc4)ccc32)cc1]

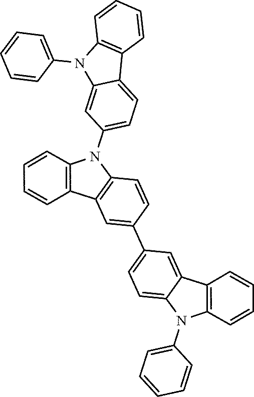 [SMILES: c1ccc(-n2c3ccccc3c3cc(-c4ccc5c(c4)c4ccccc4n5-c4ccc5c6ccccc6n(-c6ccccc6)c5c4)ccc32)cc1]

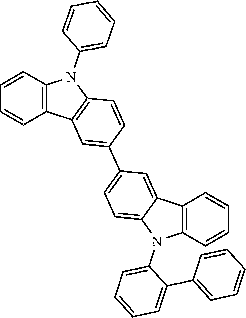 [SMILES: c1ccc(-c2ccccc2-n2c3ccccc3c3cc(-c4ccc5c(c4)c4ccccc4n5-c4ccccc4)ccc32)cc1]

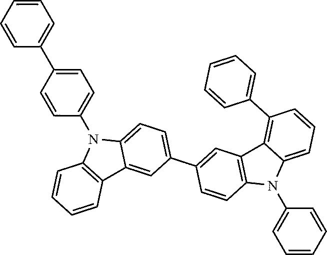 [SMILES: c1ccc(-c2ccc(-n3c4ccccc4c4cc(-c5ccc6c(c5)c5c(-c7ccccc7)cccc5n6-c5ccccc5)ccc43)cc2)cc1]

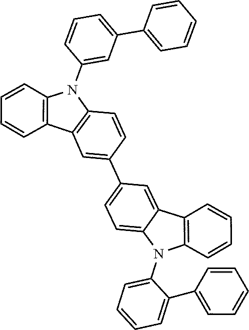 [SMILES: c1ccc(-c2cccc(-n3c4ccccc4c4cc(-c5ccc6c(c5)c5ccccc5n6-c5ccccc5-c5ccccc5)ccc43)c2)cc1]

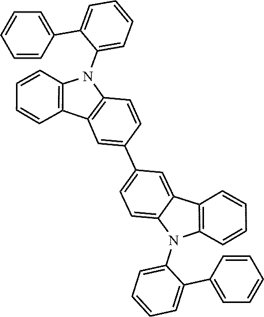 [SMILES: c1ccc(-c2ccccc2-n2c3ccccc3c3cc(-c4ccc5c(c4)c4ccccc4n5-c4ccccc4-c4ccccc4)ccc32)cc1]

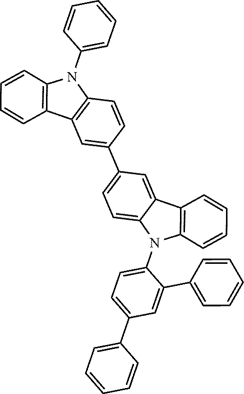 [SMILES: c1ccc(-c2ccc(-n3c4ccccc4c4cc(-c5ccc6c(c5)c5ccccc5n6-c5ccccc5)ccc43)c(-c3ccccc3)c2)cc1]

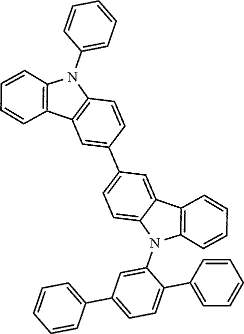 [SMILES: c1ccc(-c2ccc(-c3ccccc3)c(-n3c4ccccc4c4cc(-c5ccc6c(c5)c5ccccc5n6-c5ccccc5)ccc43)c2)cc1]

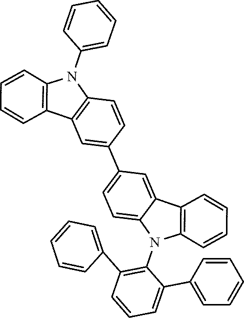 [SMILES: c1ccc(-c2cccc(-c3ccccc3)c2-n2c3ccccc3c3cc(-c4ccc5c(c4)c4ccccc4n5-c4ccccc4)ccc32)cc1]

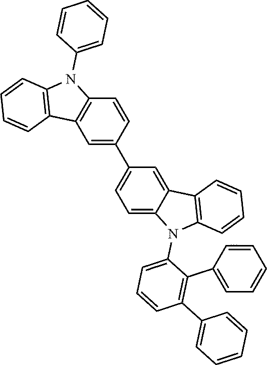 [SMILES: c1ccc(-c2cccc(-n3c4ccccc4c4cc(-c5ccc6c(c5)c5ccccc5n6-c5ccccc5)ccc43)c2-c2ccccc2)cc1]

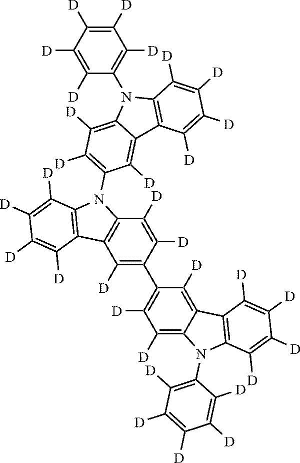 [SMILES: [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c5c(c4[2H])c4c([2H])c([2H])c([2H])c([2H])c4n5-c4c([2H])c([2H])c([2H])c([2H])c4[2H])c([2H])c([2H])c32)c([2H])c1[2H]]

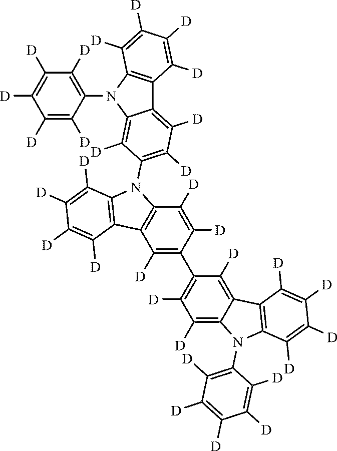 [SMILES: [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c5c6c([2H])c([2H])c([2H])c([2H])c6n(-c6c([2H])c([2H])c([2H])c([2H])c6[2H])c5c4[2H])c([2H])c([2H])c32)c([2H])c1[2H]]

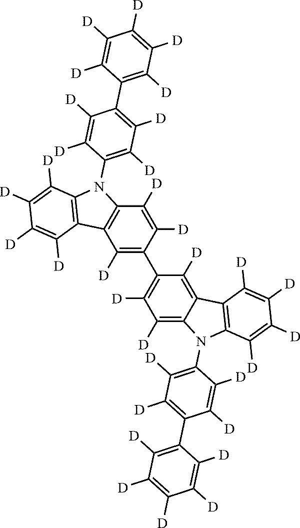 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c(-c6c([2H])c([2H])c([2H])c([2H])c6[2H])c([2H])c5[2H])c([2H])c([2H])c43)c([2H])c2[2H])c([2H])c1[2H]]

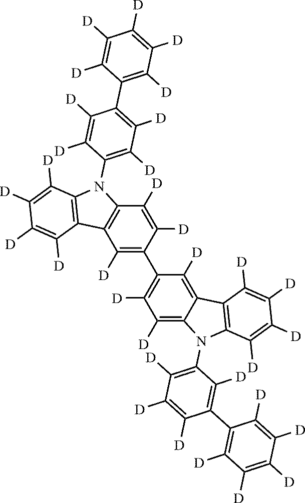 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c(-c6c([2H])c([2H])c([2H])c([2H])c6[2H])c5[2H])c([2H])c([2H])c43)c([2H])c2[2H])c([2H])c1[2H]]

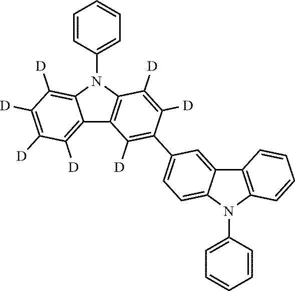 [SMILES: [2H]c1c([2H])c([2H])c2c(c1[2H])c1c([2H])c(-c3ccc4c(c3)c3ccccc3n4-c3ccccc3)c([2H])c([2H])c1n2-c1ccccc1]

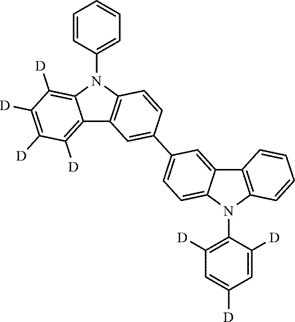 [SMILES: [2H]c1cc([2H])c(-n2c3ccccc3c3cc(-c4ccc5c(c4)c4c([2H])c([2H])c([2H])c([2H])c4n5-c4ccccc4)ccc32)c([2H])c1]

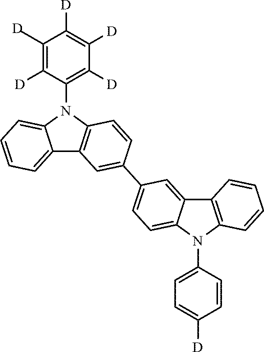

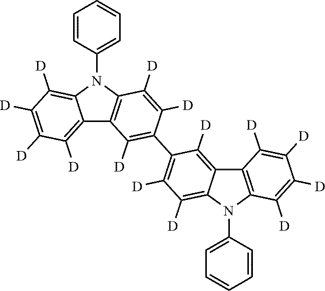 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccccc3)c([2H])c([2H])c1n2-c1ccccc1]

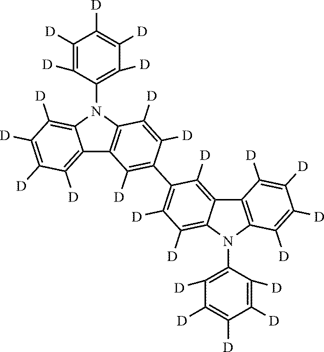 [SMILES: [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c([2H])c4[2H])c([2H])c([2H])c32)c([2H])c1[2H]]

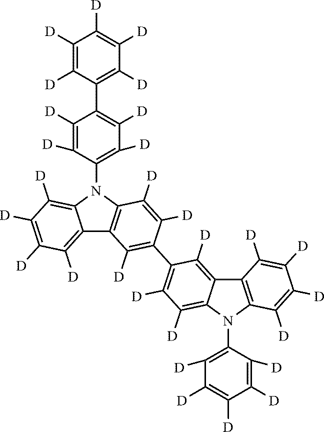 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c([2H])c5[2H])c([2H])c([2H])c43)c([2H])c2[2H])c([2H])c1[2H]]

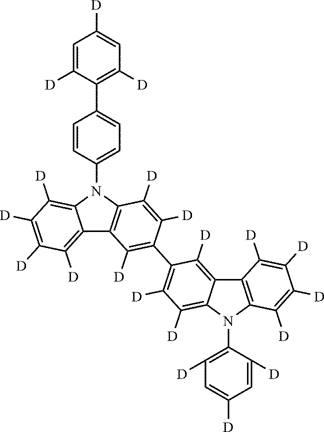 [SMILES: [2H]c1cc([2H])c(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])cc([2H])cc5[2H])c([2H])c([2H])c43)cc2)c([2H])c1]

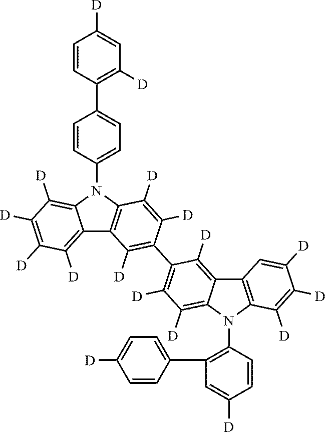 [SMILES: [2H]c1ccc(-c2cc([2H])ccc2-n2c3c([2H])c([2H])c([2H])cc3c3c([2H])c(-c4c([2H])c([2H])c5c(c4[2H])c4c([2H])c([2H])c([2H])c([2H])c4n5-c4ccc(-c5ccc([2H])cc5[2H])cc4)c([2H])c([2H])c32)cc1]

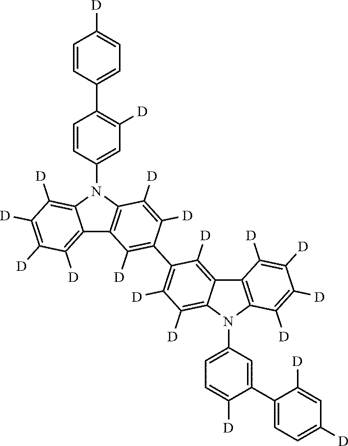 [SMILES: [2H]c1ccc(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc([2H])c(-c6ccc([2H])cc6[2H])c5)c([2H])c([2H])c43)cc2[2H])cc1]

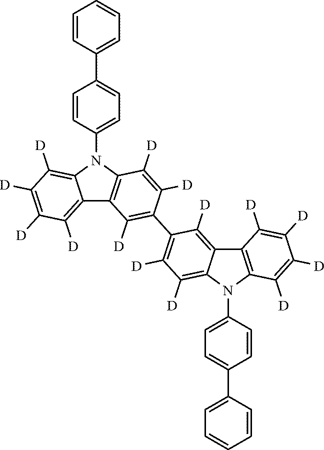 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4ccccc4)cc3)c([2H])c([2H])c1n2-c1ccc(-c2ccccc2)cc1]

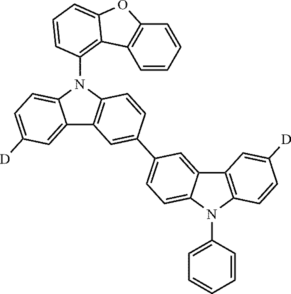 [SMILES: [2H]c1ccc2c(c1)c1cc(-c3ccc4c(c3)c3cc([2H])ccc3n4-c3cccc4oc5ccccc5c34)ccc1n2-c1ccccc1]

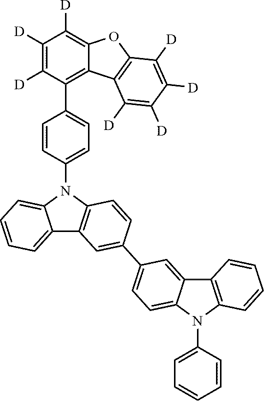 [SMILES: [2H]c1c([2H])c([2H])c2c(oc3c([2H])c([2H])c([2H])c(-c4ccc(-n5c6ccccc6c6cc(-c7ccc8c(c7)c7ccccc7n8-c7ccccc7)ccc65)cc4)c32)c1[2H]]

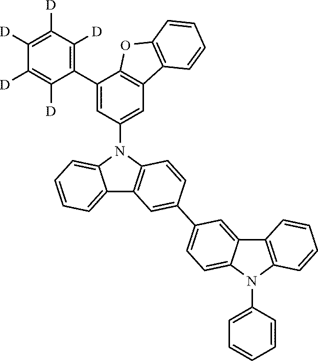 [SMILES: [2H]c1c([2H])c([2H])c(-c2cc(-n3c4ccccc4c4cc(-c5ccc6c(c5)c5ccccc5n6-c5ccccc5)ccc43)cc3c2oc2ccccc23)c([2H])c1[2H]]

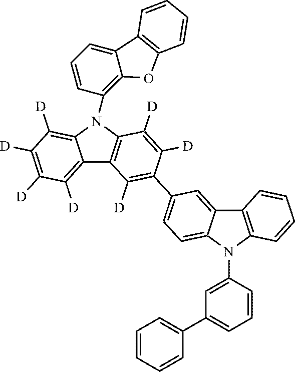 [SMILES: [2H]c1c([2H])c([2H])c2c(c1[2H])c1c([2H])c(-c3ccc4c(c3)c3ccccc3n4-c3cccc(-c4ccccc4)c3)c([2H])c([2H])c1n2-c1cccc2c1oc1ccccc12]

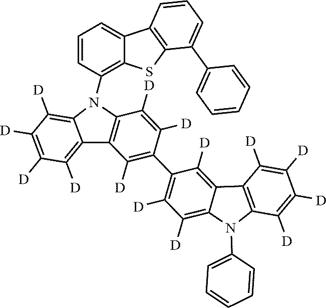 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4c3sc3c(-c5ccccc5)cccc34)c([2H])c([2H])c1n2-c1ccccc1]

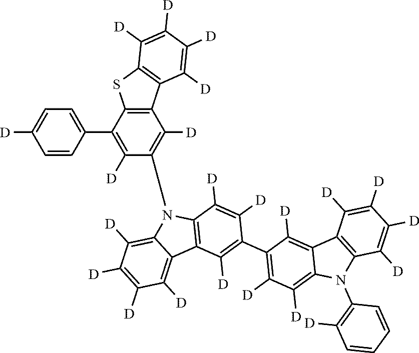 [SMILES: [2H]c1ccc(-c2c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccccc5[2H])c([2H])c([2H])c43)c([2H])c3c2sc2c([2H])c([2H])c([2H])c([2H])c23)cc1]

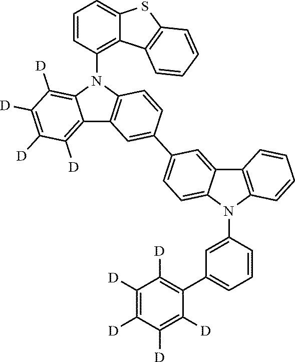 [SMILES: [2H]c1c([2H])c([2H])c(-c2cccc(-n3c4ccccc4c4cc(-c5ccc6c(c5)c5c([2H])c([2H])c([2H])c([2H])c5n6-c5cccc6sc7ccccc7c56)ccc43)c2)c([2H])c1[2H]]

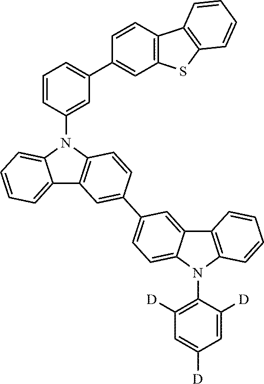 [SMILES: [2H]c1cc([2H])c(-n2c3ccccc3c3cc(-c4ccc5c(c4)c4ccccc4n5-c4cccc(-c5ccc6c(c5)sc5ccccc56)c4)ccc32)c([2H])c1]

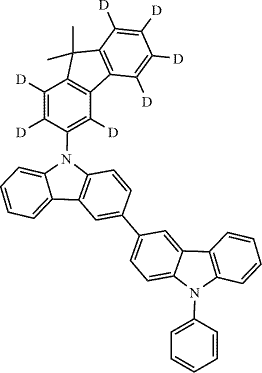 [SMILES: [2H]c1c([2H])c([2H])c2c(c1[2H])-c1c([2H])c(-n3c4ccccc4c4cc(-c5ccc6c(c5)c5ccccc5n6-c5ccccc5)ccc43)c([2H])c([2H])c1C2(C)C]

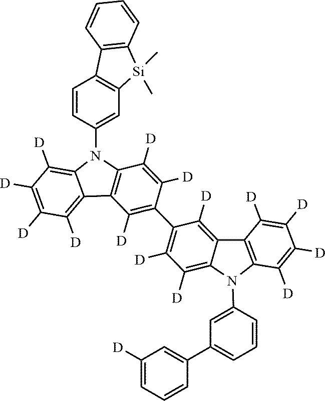 [SMILES: [2H]c1cccc(-c2cccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc6c(c5)[Si](C)(C)c5ccccc5-6)c([2H])c([2H])c43)c2)c1]

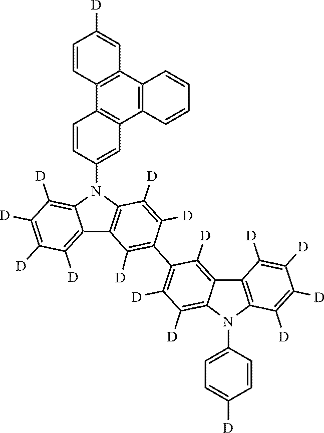 [SMILES: [2H]c1ccc(-n2c3c([2H])c([2H])c([2H])c([2H])c3c3c([2H])c(-c4c([2H])c([2H])c5c(c4[2H])c4c([2H])c([2H])c([2H])c([2H])c4n5-c4ccc5c6ccc([2H])cc6c6ccccc6c5c4)c([2H])c([2H])c32)cc1]

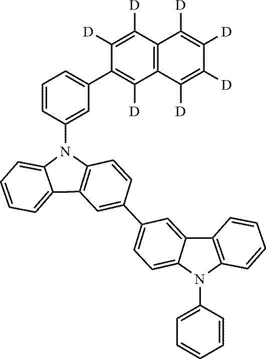 [SMILES: [2H]c1c([2H])c([2H])c2c([2H])c(-c3cccc(-n4c5ccccc5c5cc(-c6ccc7c(c6)c6ccccc6n7-c6ccccc6)ccc54)c3)c([2H])c([2H])c2c1[2H]]

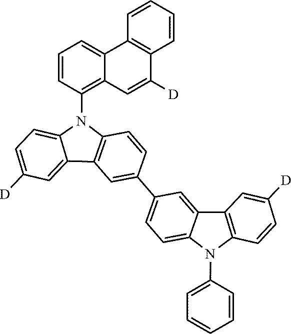 [SMILES: [2H]c1ccc2c(c1)c1cc(-c3ccc4c(c3)c3cc([2H])ccc3n4-c3cccc4c3cc([2H])c3ccccc34)ccc1n2-c1ccccc1]

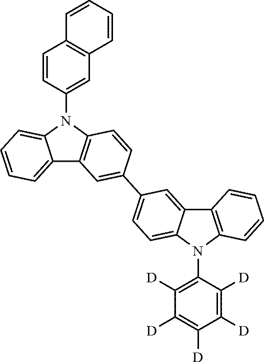 [SMILES: [2H]c1c([2H])c([2H])c(-n2c3ccccc3c3cc(-c4ccc5c(c4)c4ccccc4n5-c4ccc5ccccc5c4)ccc32)c([2H])c1[2H]]

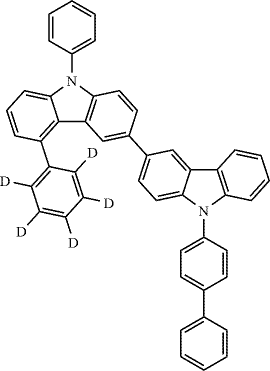 [SMILES: [2H]c1c([2H])c([2H])c(-c2cccc3c2c2cc(-c4ccc5c(c4)c4ccccc4n5-c4ccc(-c5ccccc5)cc4)ccc2n3-c2ccccc2)c([2H])c1[2H]]

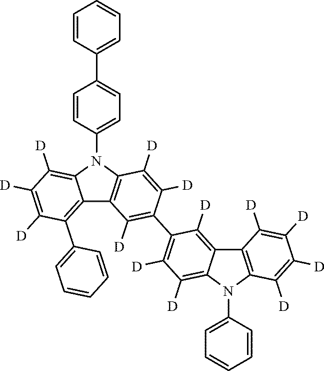 [SMILES: [2H]c1c([2H])c([2H])c2c(c1[2H])c1c([2H])c(-c3c([2H])c([2H])c4c(c3[2H])c3c(-c5ccccc5)c([2H])c([2H])c([2H])c3n4-c3ccc(-c4ccccc4)cc3)c([2H])c([2H])c1n2-c1ccccc1]

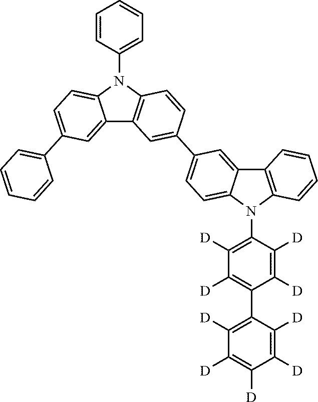 [SMILES: [2H]c1c([2H])c([2H])c(-c2c([2H])c([2H])c(-n3c4ccccc4c4cc(-c5ccc6c(c5)c5cc(-c7ccccc7)ccc5n6-c5ccccc5)ccc43)c([2H])c2[2H])c([2H])c1[2H]]

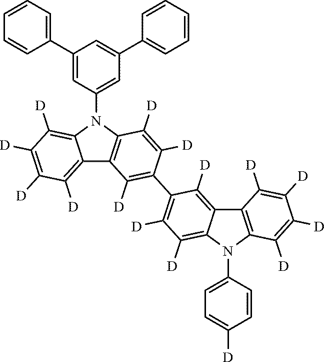 [SMILES: [2H]c1ccc(-n2c3c([2H])c([2H])c([2H])c([2H])c3c3c([2H])c(-c4c([2H])c([2H])c5c(c4[2H])c4c([2H])c([2H])c([2H])c([2H])c4n5-c4cc(-c5ccccc5)cc(-c5ccccc5)c4)c([2H])c([2H])c32)cc1]

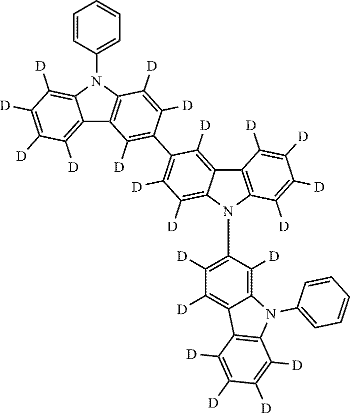 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c4c5c([2H])c([2H])c([2H])c([2H])c5n(-c5ccccc5)c4c3[2H])c([2H])c([2H])c1n2-c1ccccc1]

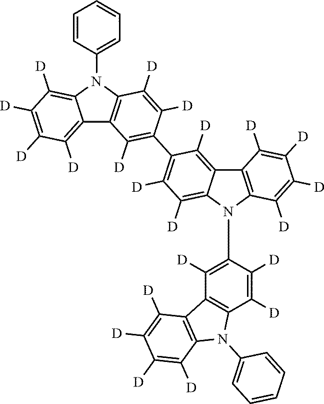 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c4c(c3[2H])c3c([2H])c([2H])c([2H])c([2H])c3n4-c3ccccc3)c([2H])c([2H])c1n2-c1ccccc1]

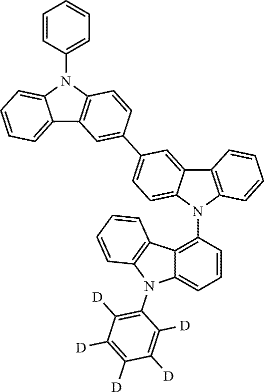 [SMILES: [2H]c1c([2H])c([2H])c(-n2c3ccccc3c3c(-n4c5ccccc5c5cc(-c6ccc7c(c6)c6ccccc6n7-c6ccccc6)ccc54)cccc32)c([2H])c1[2H]]

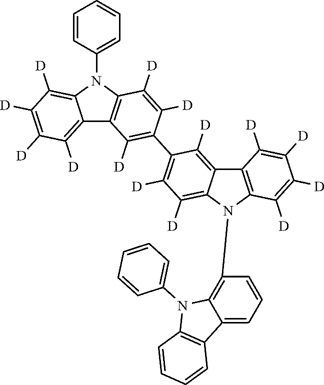 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4c5ccccc5n(-c5ccccc5)c34)c([2H])c([2H])c1n2-c1ccccc1]

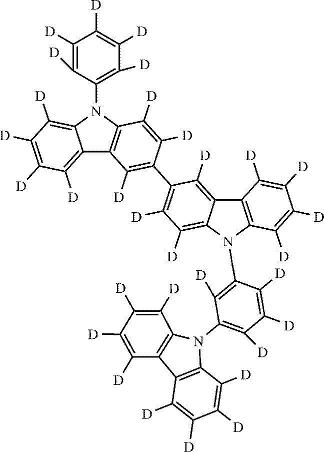 [SMILES: [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c(-n5c6c([2H])c([2H])c([2H])c([2H])c6c6c([2H])c([2H])c([2H])c([2H])c65)c4[2H])c([2H])c([2H])c32)c([2H])c1[2H]]

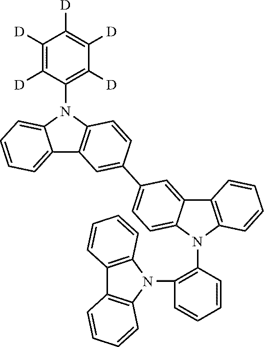 [SMILES: [2H]c1c([2H])c([2H])c(-n2c3ccccc3c3cc(-c4ccc5c(c4)c4ccccc4n5-c4ccccc4-n4c5ccccc5c5ccccc54)ccc32)c([2H])c1[2H]]

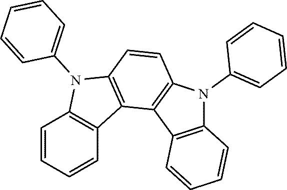 [SMILES: c1ccc(-n2c3ccccc3c3c4c5ccccc5n(-c5ccccc5)c4ccc32)cc1]

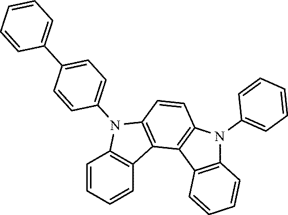 [SMILES: c1ccc(-c2ccc(-n3c4ccccc4c4c5c6ccccc6n(-c6ccccc6)c5ccc43)cc2)cc1]

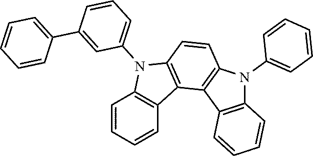 [SMILES: c1ccc(-c2cccc(-n3c4ccccc4c4c5c6ccccc6n(-c6ccccc6)c5ccc43)c2)cc1]

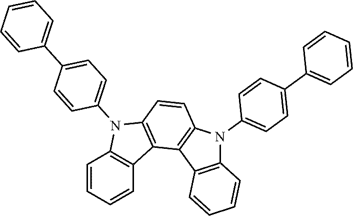 [SMILES: c1ccc(-c2ccc(-n3c4ccccc4c4c5c6ccccc6n(-c6ccc(-c7ccccc7)cc6)c5ccc43)cc2)cc1]

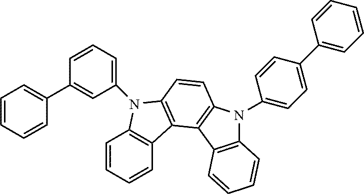 [SMILES: c1ccc(-c2ccc(-n3c4ccccc4c4c5c6ccccc6n(-c6cccc(-c7ccccc7)c6)c5ccc43)cc2)cc1]

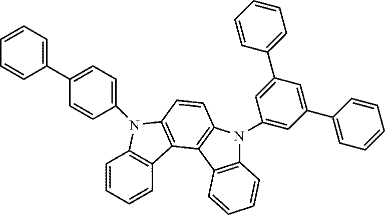 [SMILES: c1ccc(-c2ccc(-n3c4ccccc4c4c5c6ccccc6n(-c6cc(-c7ccccc7)cc(-c7ccccc7)c6)c5ccc43)cc2)cc1]

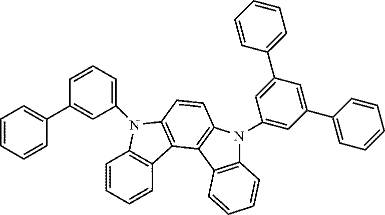 [SMILES: c1ccc(-c2cccc(-n3c4ccccc4c4c5c6ccccc6n(-c6cc(-c7ccccc7)cc(-c7ccccc7)c6)c5ccc43)c2)cc1]

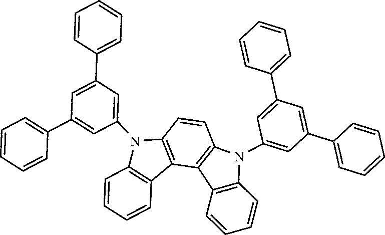 [SMILES: c1ccc(-c2cc(-c3ccccc3)cc(-n3c4ccccc4c4c5c6ccccc6n(-c6cc(-c7ccccc7)cc(-c7ccccc7)c6)c5ccc43)c2)cc1]

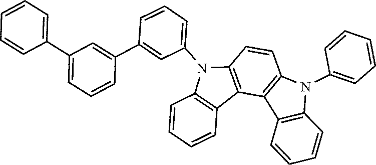 [SMILES: c1ccc(-c2cccc(-c3cccc(-n4c5ccccc5c5c6c7ccccc7n(-c7ccccc7)c6ccc54)c3)c2)cc1]

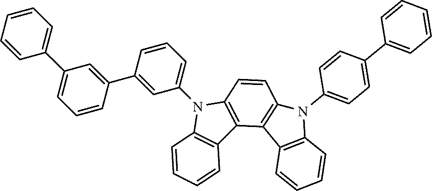 [SMILES: c1ccc(-c2ccc(-n3c4ccccc4c4c5c6ccccc6n(-c6cccc(-c7cccc(-c8ccccc8)c7)c6)c5ccc43)cc2)cc1]

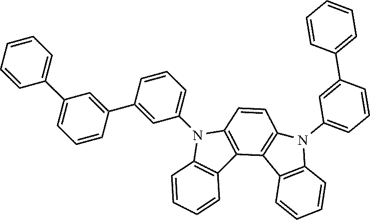 [SMILES: c1ccc(-c2cccc(-c3cccc(-n4c5ccccc5c5c6c7ccccc7n(-c7cccc(-c8ccccc8)c7)c6ccc54)c3)c2)cc1]

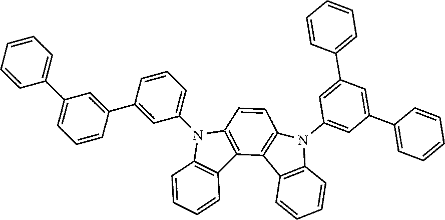 [SMILES: c1ccc(-c2cccc(-c3cccc(-n4c5ccccc5c5c6c7ccccc7n(-c7cc(-c8ccccc8)cc(-c8ccccc8)c7)c6ccc54)c3)c2)cc1]

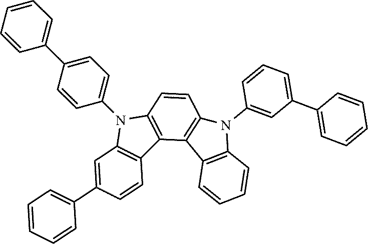 [SMILES: c1ccc(-c2ccc(-n3c4cc(-c5ccccc5)ccc4c4c5c6ccccc6n(-c6cccc(-c7ccccc7)c6)c5ccc43)cc2)cc1]

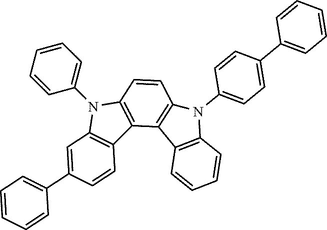 [SMILES: c1ccc(-c2ccc(-n3c4ccccc4c4c5c6ccc(-c7ccccc7)cc6n(-c6ccccc6)c5ccc43)cc2)cc1]

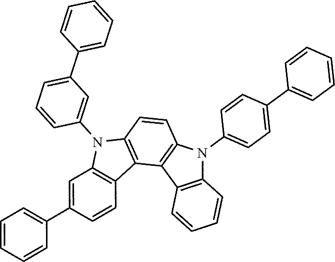 [SMILES: c1ccc(-c2ccc(-n3c4ccccc4c4c5c6ccc(-c7ccccc7)cc6n(-c6cccc(-c7ccccc7)c6)c5ccc43)cc2)cc1]

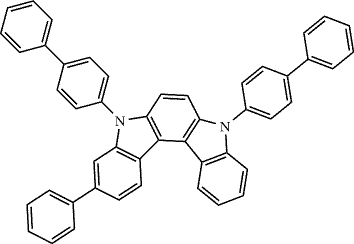 [SMILES: c1ccc(-c2ccc(-n3c4ccccc4c4c5c6ccc(-c7ccccc7)cc6n(-c6ccc(-c7ccccc7)cc6)c5ccc43)cc2)cc1]

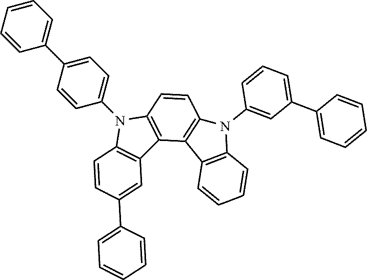 [SMILES: c1ccc(-c2ccc(-n3c4ccc(-c5ccccc5)cc4c4c5c6ccccc6n(-c6cccc(-c7ccccc7)c6)c5ccc43)cc2)cc1]

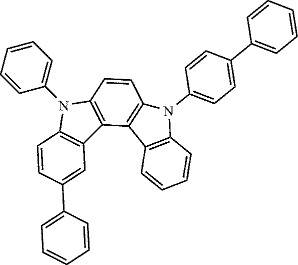 [SMILES: c1ccc(-c2ccc(-n3c4ccccc4c4c5c6cc(-c7ccccc7)ccc6n(-c6ccccc6)c5ccc43)cc2)cc1]

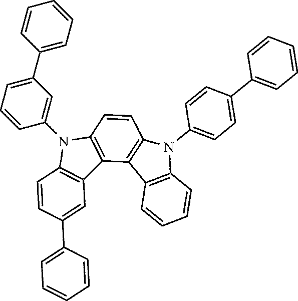 [SMILES: c1ccc(-c2ccc(-n3c4ccccc4c4c5c6cc(-c7ccccc7)ccc6n(-c6cccc(-c7ccccc7)c6)c5ccc43)cc2)cc1]

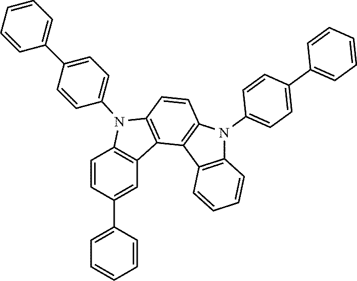 [SMILES: c1ccc(-c2ccc(-n3c4ccccc4c4c5c6cc(-c7ccccc7)ccc6n(-c6ccc(-c7ccccc7)cc6)c5ccc43)cc2)cc1]

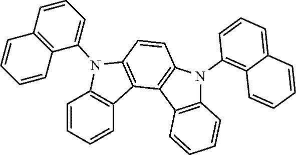 [SMILES: c1ccc2c(-n3c4ccccc4c4c5c6ccccc6n(-c6cccc7ccccc67)c5ccc43)cccc2c1]

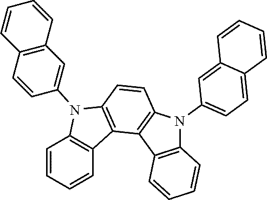 [SMILES: c1ccc2cc(-n3c4ccccc4c4c5c6ccccc6n(-c6ccc7ccccc7c6)c5ccc43)ccc2c1]

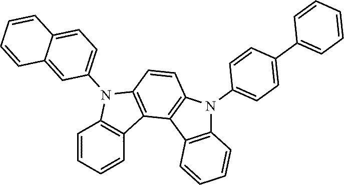 [SMILES: c1ccc(-c2ccc(-n3c4ccccc4c4c5c6ccccc6n(-c6ccc7ccccc7c6)c5ccc43)cc2)cc1]

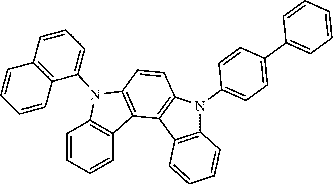 [SMILES: c1ccc(-c2ccc(-n3c4ccccc4c4c5c6ccccc6n(-c6cccc7ccccc67)c5ccc43)cc2)cc1]

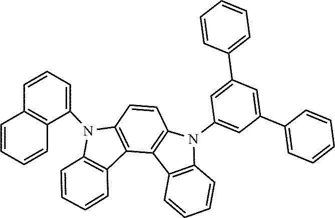 [SMILES: c1ccc(-c2cc(-c3ccccc3)cc(-n3c4ccccc4c4c5c6ccccc6n(-c6cccc7ccccc67)c5ccc43)c2)cc1]

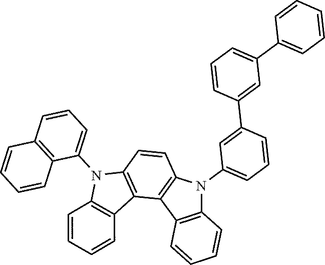 [SMILES: c1ccc(-c2cccc(-c3cccc(-n4c5ccccc5c5c6c7ccccc7n(-c7cccc8ccccc78)c6ccc54)c3)c2)cc1]

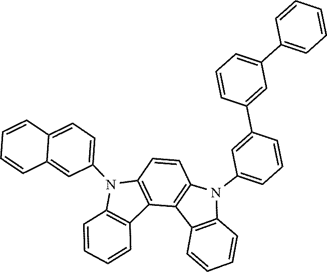 [SMILES: c1ccc(-c2cccc(-c3cccc(-n4c5ccccc5c5c6c7ccccc7n(-c7ccc8ccccc8c7)c6ccc54)c3)c2)cc1]

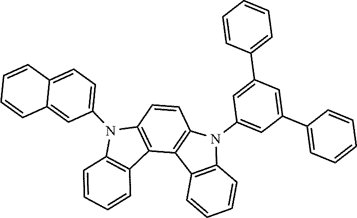 [SMILES: c1ccc(-c2cc(-c3ccccc3)cc(-n3c4ccccc4c4c5c6ccccc6n(-c6ccc7ccccc7c6)c5ccc43)c2)cc1]

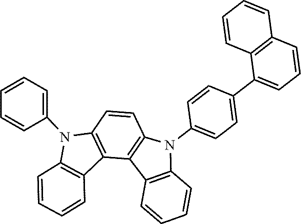 [SMILES: c1ccc(-n2c3ccccc3c3c4c5ccccc5n(-c5ccc(-c6cccc7ccccc67)cc5)c4ccc32)cc1]

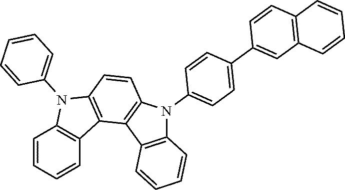 [SMILES: c1ccc(-n2c3ccccc3c3c4c5ccccc5n(-c5ccc(-c6ccc7ccccc7c6)cc5)c4ccc32)cc1]

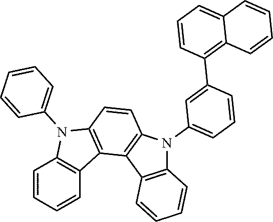 [SMILES: c1ccc(-n2c3ccccc3c3c4c5ccccc5n(-c5cccc(-c6cccc7ccccc67)c5)c4ccc32)cc1]

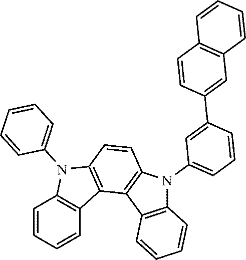 [SMILES: c1ccc(-n2c3ccccc3c3c4c5ccccc5n(-c5cccc(-c6ccc7ccccc7c6)c5)c4ccc32)cc1]

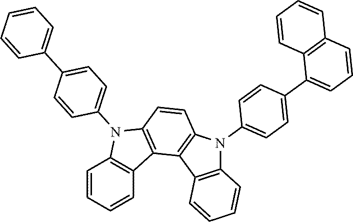 [SMILES: c1ccc(-c2ccc(-n3c4ccccc4c4c5c6ccccc6n(-c6ccc(-c7cccc8ccccc78)cc6)c5ccc43)cc2)cc1]

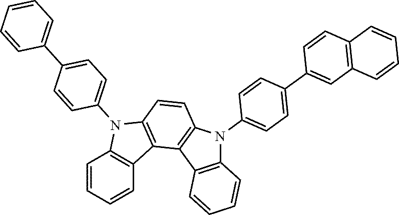 [SMILES: c1ccc(-c2ccc(-n3c4ccccc4c4c5c6ccccc6n(-c6ccc(-c7ccc8ccccc8c7)cc6)c5ccc43)cc2)cc1]

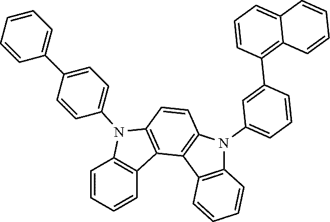 [SMILES: c1ccc(-c2ccc(-n3c4ccccc4c4c5c6ccccc6n(-c6cccc(-c7cccc8ccccc78)c6)c5ccc43)cc2)cc1]

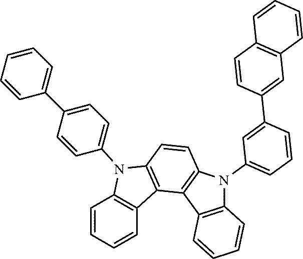 [SMILES: c1ccc(-c2ccc(-n3c4ccccc4c4c5c6ccccc6n(-c6cccc(-c7ccc8ccccc8c7)c6)c5ccc43)cc2)cc1]

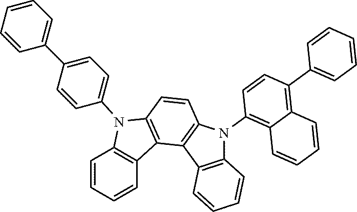 [SMILES: c1ccc(-c2ccc(-n3c4ccccc4c4c5c6ccccc6n(-c6ccc(-c7ccccc7)c7ccccc67)c5ccc43)cc2)cc1]

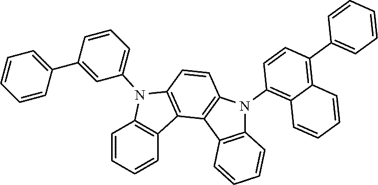 [SMILES: c1ccc(-c2cccc(-n3c4ccccc4c4c5c6ccccc6n(-c6ccc(-c7ccccc7)c7ccccc67)c5ccc43)c2)cc1]

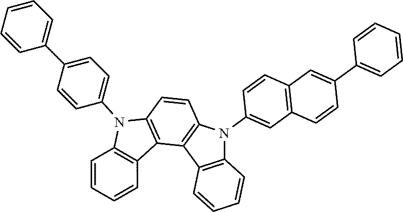 [SMILES: c1ccc(-c2ccc(-n3c4ccccc4c4c5c6ccccc6n(-c6ccc7cc(-c8ccccc8)ccc7c6)c5ccc43)cc2)cc1]

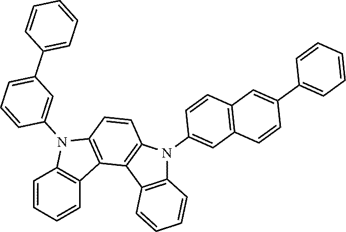 [SMILES: c1ccc(-c2cccc(-n3c4ccccc4c4c5c6ccccc6n(-c6ccc7cc(-c8ccccc8)ccc7c6)c5ccc43)c2)cc1]

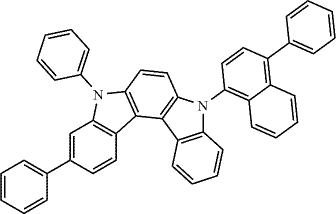 [SMILES: c1ccc(-c2ccc3c4c5c6ccccc6n(-c6ccc(-c7ccccc7)c7ccccc67)c5ccc4n(-c4ccccc4)c3c2)cc1]

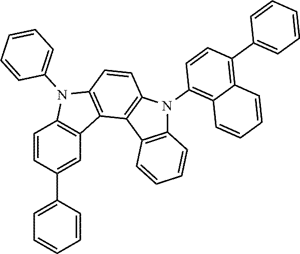 [SMILES: c1ccc(-c2ccc3c(c2)c2c4c5ccccc5n(-c5ccc(-c6ccccc6)c6ccccc56)c4ccc2n3-c2ccccc2)cc1]

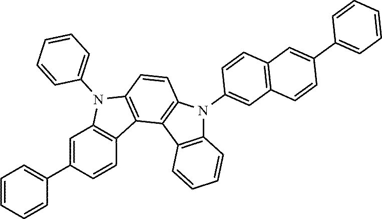 [SMILES: c1ccc(-c2ccc3cc(-n4c5ccccc5c5c6c7ccc(-c8ccccc8)cc7n(-c7ccccc7)c6ccc54)ccc3c2)cc1]

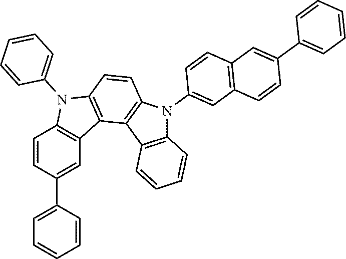 [SMILES: c1ccc(-c2ccc3cc(-n4c5ccccc5c5c6c7cc(-c8ccccc8)ccc7n(-c7ccccc7)c6ccc54)ccc3c2)cc1]

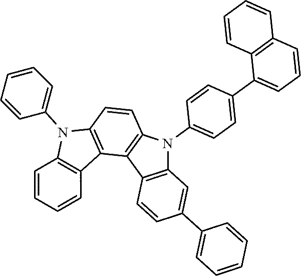 [SMILES: c1ccc(-c2ccc3c4c5c6ccccc6n(-c6ccccc6)c5ccc4n(-c4ccc(-c5cccc6ccccc56)cc4)c3c2)cc1]

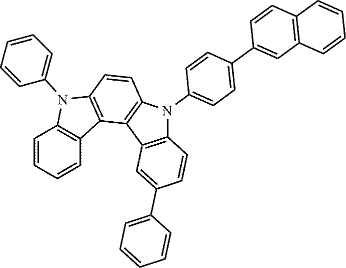 [SMILES: c1ccc(-c2ccc3c(c2)c2c4c5ccccc5n(-c5ccccc5)c4ccc2n3-c2ccc(-c3ccc4ccccc4c3)cc2)cc1]

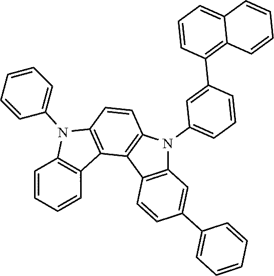 [SMILES: c1ccc(-c2ccc3c4c5c6ccccc6n(-c6ccccc6)c5ccc4n(-c4cccc(-c5cccc6ccccc56)c4)c3c2)cc1]

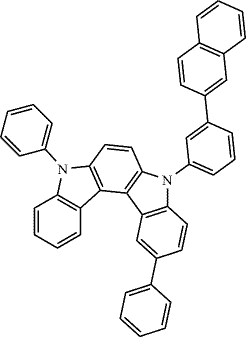 [SMILES: c1ccc(-c2ccc3c(c2)c2c4c5ccccc5n(-c5ccccc5)c4ccc2n3-c2cccc(-c3ccc4ccccc4c3)c2)cc1]

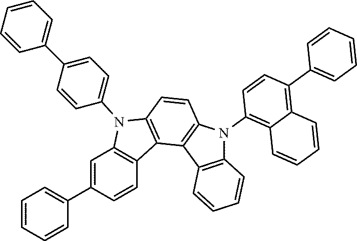 [SMILES: c1ccc(-c2ccc(-n3c4cc(-c5ccccc5)ccc4c4c5c6ccccc6n(-c6ccc(-c7ccccc7)c7ccccc67)c5ccc43)cc2)cc1]

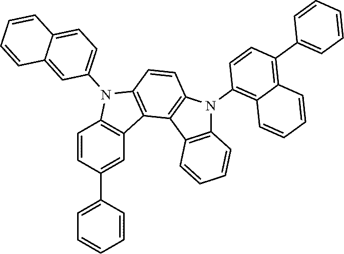 [SMILES: c1ccc(-c2ccc3c(c2)c2c4c5ccccc5n(-c5ccc(-c6ccccc6)c6ccccc56)c4ccc2n3-c2ccc3ccccc3c2)cc1]

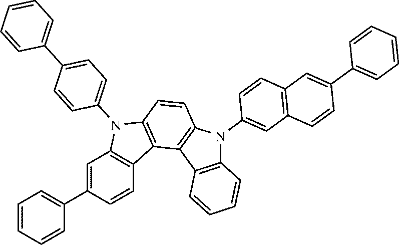 [SMILES: c1ccc(-c2ccc(-n3c4cc(-c5ccccc5)ccc4c4c5c6ccccc6n(-c6ccc7cc(-c8ccccc8)ccc7c6)c5ccc43)cc2)cc1]

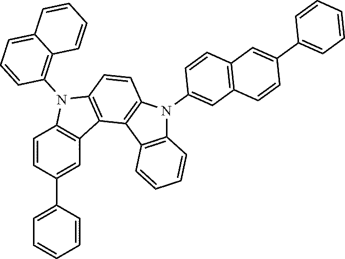 [SMILES: c1ccc(-c2ccc3cc(-n4c5ccccc5c5c6c7cc(-c8ccccc8)ccc7n(-c7cccc8ccccc78)c6ccc54)ccc3c2)cc1]

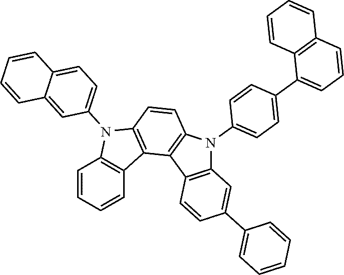 [SMILES: c1ccc(-c2ccc3c4c5c6ccccc6n(-c6ccc7ccccc7c6)c5ccc4n(-c4ccc(-c5cccc6ccccc56)cc4)c3c2)cc1]

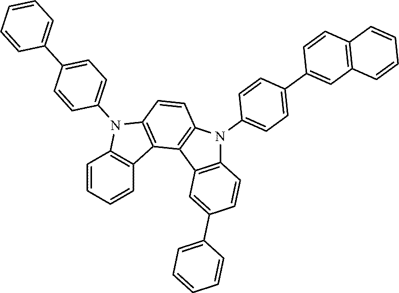 [SMILES: c1ccc(-c2ccc(-n3c4ccccc4c4c5c6cc(-c7ccccc7)ccc6n(-c6ccc(-c7ccc8ccccc8c7)cc6)c5ccc43)cc2)cc1]

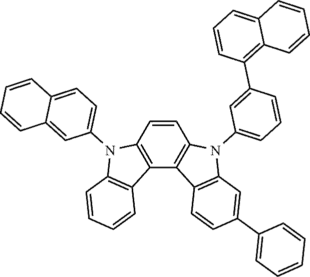 [SMILES: c1ccc(-c2ccc3c4c5c6ccccc6n(-c6ccc7ccccc7c6)c5ccc4n(-c4cccc(-c5cccc6ccccc56)c4)c3c2)cc1]

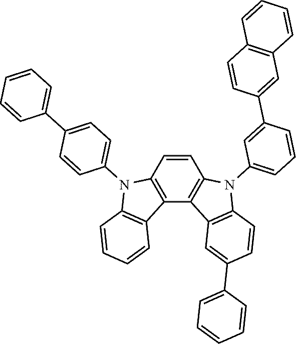 [SMILES: c1ccc(-c2ccc(-n3c4ccccc4c4c5c6cc(-c7ccccc7)ccc6n(-c6cccc(-c7ccc8ccccc8c7)c6)c5ccc43)cc2)cc1]

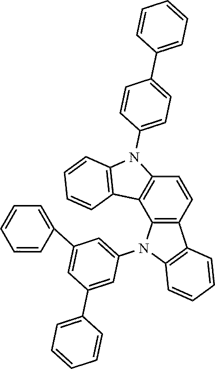 [SMILES: c1ccc(-c2ccc(-n3c4ccccc4c4c3ccc3c5ccccc5n(-c5cc(-c6ccccc6)cc(-c6ccccc6)c5)c34)cc2)cc1]

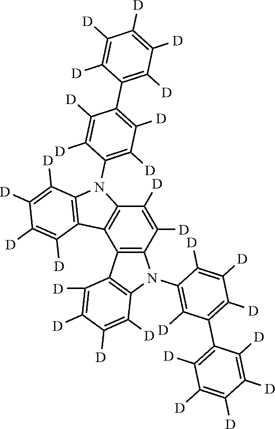 [SMILES: [2H]c1c([2H])c([2H])c(-c2c([2H])c([2H])c(-n3c4c([2H])c([2H])c([2H])c([2H])c4c4c5c6c([2H])c([2H])c([2H])c([2H])c6n(-c6c([2H])c([2H])c([2H])c(-c7c([2H])c([2H])c([2H])c([2H])c7[2H])c6[2H])c5c([2H])c([2H])c43)c([2H])c2[2H])c([2H])c1[2H]]

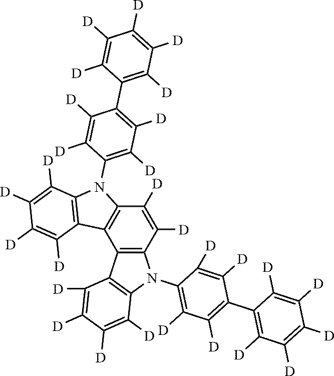 [SMILES: [2H]c1c([2H])c([2H])c(-c2c([2H])c([2H])c(-n3c4c([2H])c([2H])c([2H])c([2H])c4c4c5c6c([2H])c([2H])c([2H])c([2H])c6n(-c6c([2H])c([2H])c(-c7c([2H])c([2H])c([2H])c([2H])c7[2H])c([2H])c6[2H])c5c([2H])c([2H])c43)c([2H])c2[2H])c([2H])c1[2H]]

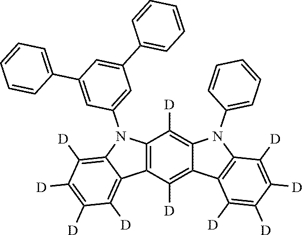 [SMILES: [2H]c1c([2H])c([2H])c2c(c1[2H])c1c([2H])c3c4c([2H])c([2H])c([2H])c([2H])c4n(-c4cc(-c5ccccc5)cc(-c5ccccc5)c4)c3c([2H])c1n2-c1ccccc1]

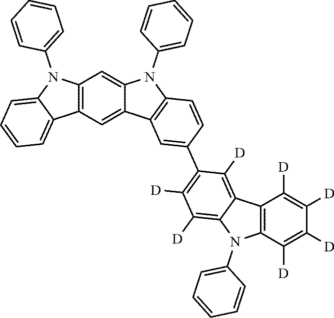 [SMILES: [2H]c1c([2H])c([2H])c2c(c1[2H])c1c([2H])c(-c3ccc4c(c3)c3cc5c6ccccc6n(-c6ccccc6)c5cc3n4-c3ccccc3)c([2H])c([2H])c1n2-c1ccccc1]

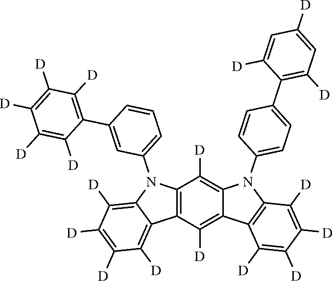 [SMILES: [2H]c1cc([2H])c(-c2ccc(-n3c4c([2H])c([2H])c([2H])c([2H])c4c4c([2H])c5c6c([2H])c([2H])c([2H])c([2H])c6n(-c6cccc(-c7c([2H])c([2H])c([2H])c([2H])c7[2H])c6)c5c([2H])c43)cc2)c([2H])c1]

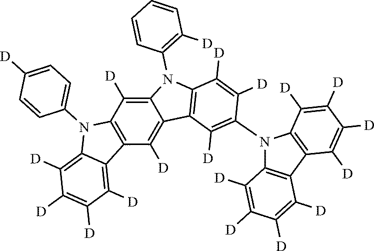 [SMILES: [2H]c1ccc(-n2c3c([2H])c([2H])c([2H])c([2H])c3c3c([2H])c4c5c([2H])c(-n6c7c([2H])c([2H])c([2H])c([2H])c7c7c([2H])c([2H])c([2H])c([2H])c76)c([2H])c([2H])c5n(-c5ccccc5[2H])c4c([2H])c32)cc1]

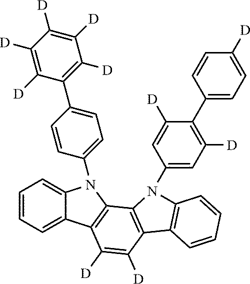 [SMILES: [2H]c1ccc(-c2c([2H])cc(-n3c4ccccc4c4c([2H])c([2H])c5c6ccccc6n(-c6ccc(-c7c([2H])c([2H])c([2H])c([2H])c7[2H])cc6)c5c43)cc2[2H])cc1]

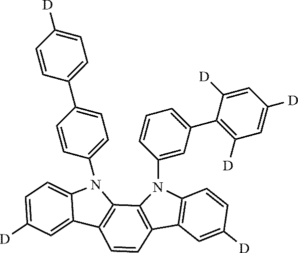 [SMILES: [2H]c1ccc(-c2ccc(-n3c4ccc([2H])cc4c4ccc5c6cc([2H])ccc6n(-c6cccc(-c7c([2H])cc([2H])cc7[2H])c6)c5c43)cc2)cc1]

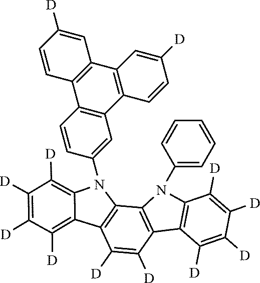 [SMILES: [2H]c1ccc2c3ccc(-n4c5c([2H])c([2H])c([2H])c([2H])c5c5c([2H])c([2H])c6c7c([2H])c([2H])c([2H])c([2H])c7n(-c7ccccc7)c6c54)cc3c3ccc([2H])cc3c2c1]

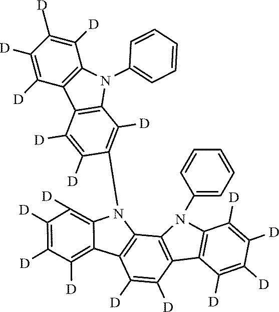

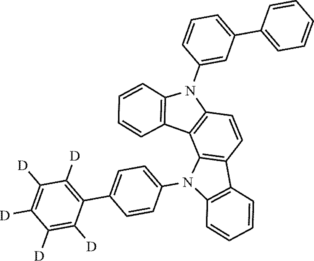 [SMILES: [2H]c1c([2H])c([2H])c(-c2ccc(-n3c4ccccc4c4ccc5c(c6ccccc6n5-c5cccc(-c6ccccc6)c5)c43)cc2)c([2H])c1[2H]]

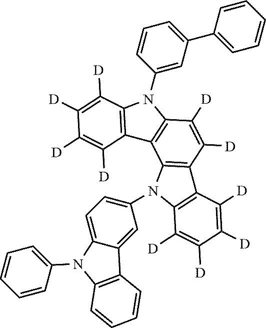 [SMILES: [2H]c1c([2H])c([2H])c2c(c1[2H])c1c(c([2H])c([2H])c3c4c([2H])c([2H])c([2H])c([2H])c4n(-c4ccc5c(c4)c4ccccc4n5-c4ccccc4)c31)n2-c1cccc(-c2ccccc2)c1]

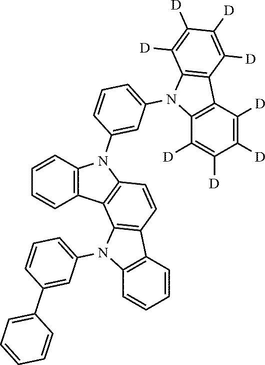 [SMILES: [2H]c1c([2H])c([2H])c2c(c1[2H])c1c([2H])c([2H])c([2H])c([2H])c1n2-c1cccc(-n2c3ccccc3c3c2ccc2c4ccccc4n(-c4cccc(-c5ccccc5)c4)c23)c1]

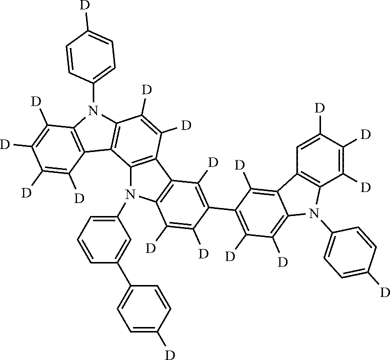 [SMILES: [2H]c1ccc(-c2cccc(-n3c4c([2H])c([2H])c(-c5c([2H])c([2H])c6c(c5[2H])c5cc([2H])c([2H])c([2H])c5n6-c5ccc([2H])cc5)c([2H])c4c4c([2H])c([2H])c5c(c6c([2H])c([2H])c([2H])c([2H])c6n5-c5ccc([2H])cc5)c43)c2)cc1]

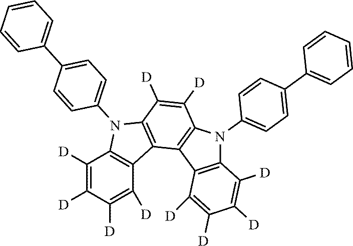 [SMILES: [2H]c1c([2H])c([2H])c2c(c1[2H])c1c3c4c([2H])c([2H])c([2H])c([2H])c4n(-c4ccc(-c5ccccc5)cc4)c3c([2H])c([2H])c1n2-c1ccc(-c2ccccc2)cc1]

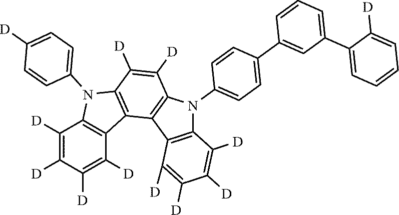 [SMILES: [2H]c1ccc(-n2c3c([2H])c([2H])c([2H])c([2H])c3c3c4c5c([2H])c([2H])c([2H])c([2H])c5n(-c5ccc(-c6cccc(-c7ccccc7[2H])c6)cc5)c4c([2H])c([2H])c32)cc1]

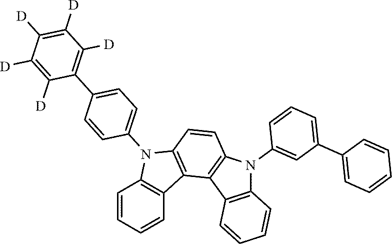 [SMILES: [2H]c1c([2H])c([2H])c(-c2ccc(-n3c4ccccc4c4c5c6ccccc6n(-c6cccc(-c7ccccc7)c6)c5ccc43)cc2)c([2H])c1[2H]]

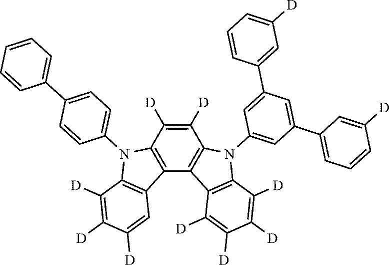 [SMILES: [2H]c1cccc(-c2cc(-c3cccc([2H])c3)cc(-n3c4c([2H])c([2H])c([2H])c([2H])c4c4c5c6cc([2H])c([2H])c([2H])c6n(-c6ccc(-c7ccccc7)cc6)c5c([2H])c([2H])c43)c2)c1]

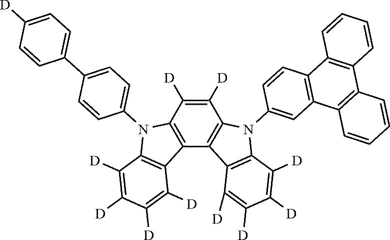 [SMILES: [2H]c1ccc(-c2ccc(-n3c4c([2H])c([2H])c([2H])c([2H])c4c4c5c6c([2H])c([2H])c([2H])c([2H])c6n(-c6ccc7c8ccccc8c8ccccc8c7c6)c5c([2H])c([2H])c43)cc2)cc1]

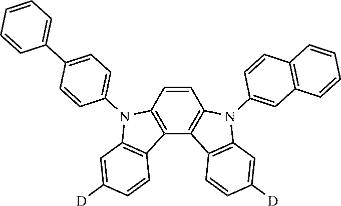 [SMILES: [2H]c1ccc2c3c4c5ccc([2H])cc5n(-c5ccc6ccccc6c5)c4ccc3n(-c3ccc(-c4ccccc4)cc3)c2c1]

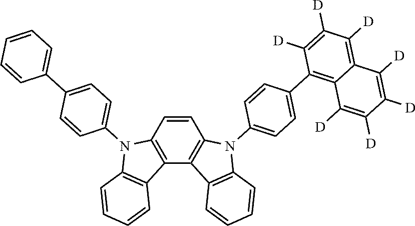 [SMILES: [2H]c1c([2H])c([2H])c2c(-c3ccc(-n4c5ccccc5c5c6c7ccccc7n(-c7ccc(-c8ccccc8)cc7)c6ccc54)cc3)c([2H])c([2H])c([2H])c2c1[2H]]

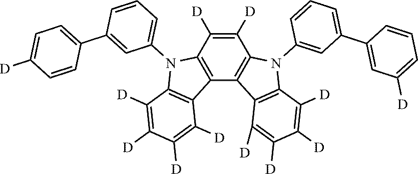 [SMILES: [2H]c1ccc(-c2cccc(-n3c4c([2H])c([2H])c([2H])c([2H])c4c4c5c6c([2H])c([2H])c([2H])c([2H])c6n(-c6cccc(-c7cccc([2H])c7)c6)c5c([2H])c([2H])c43)c2)cc1]

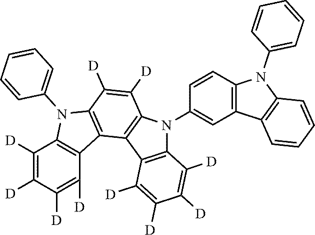 [SMILES: [2H]c1c([2H])c([2H])c2c(c1[2H])c1c3c4c([2H])c([2H])c([2H])c([2H])c4n(-c4ccc5c(c4)c4ccccc4n5-c4ccccc4)c3c([2H])c([2H])c1n2-c1ccccc1]

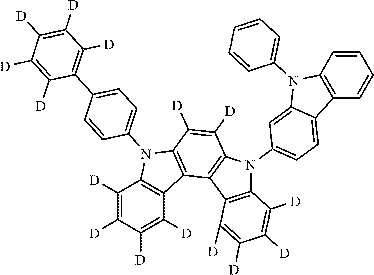 [SMILES: [2H]c1c([2H])c([2H])c(-c2ccc(-n3c4c([2H])c([2H])c([2H])c([2H])c4c4c5c6c([2H])c([2H])c([2H])c([2H])c6n(-c6ccc7c8ccccc8n(-c8ccccc8)c7c6)c5c([2H])c([2H])c43)cc2)c([2H])c1[2H]]

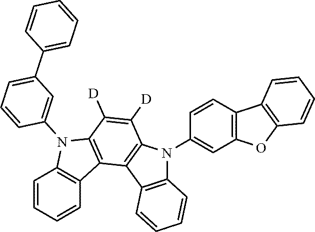 [SMILES: [2H]c1c([2H])c2c(c3ccccc3n2-c2ccc3c(c2)oc2ccccc23)c2c3ccccc3n(-c3cccc(-c4ccccc4)c3)c12]

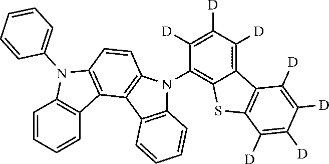 [SMILES: [2H]c1c([2H])c([2H])c2c(sc3c(-n4c5ccccc5c5c6c7ccccc7n(-c7ccccc7)c6ccc54)c([2H])c([2H])c([2H])c32)c1[2H]]

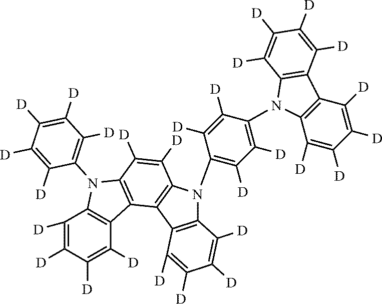 [SMILES: [2H]c1c([2H])c([2H])c(-n2c3c([2H])c([2H])c([2H])c([2H])c3c3c4c5c([2H])c([2H])c([2H])c([2H])c5n(-c5c([2H])c([2H])c(-n6c7c([2H])c([2H])c([2H])c([2H])c7c7c([2H])c([2H])c([2H])c([2H])c76)c([2H])c5[2H])c4c([2H])c([2H])c32)c([2H])c1[2H]]

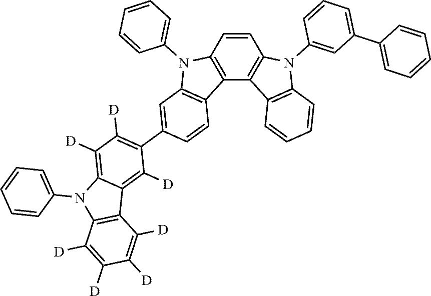 [SMILES: [2H]c1c([2H])c([2H])c2c(c1[2H])c1c([2H])c(-c3ccc4c5c6c7ccccc7n(-c7cccc(-c8ccccc8)c7)c6ccc5n(-c5ccccc5)c4c3)c([2H])c([2H])c1n2-c1ccccc1]

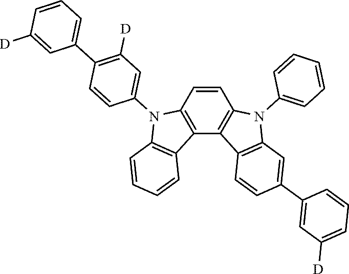 [SMILES: [2H]c1cccc(-c2ccc3c4c5c6ccccc6n(-c6ccc(-c7cccc([2H])c7)c([2H])c6)c5ccc4n(-c4ccccc4)c3c2)c1]

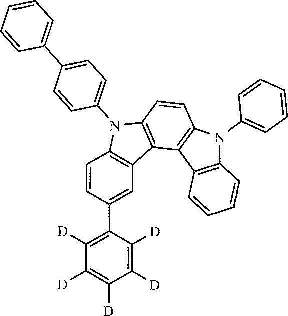 [SMILES: [2H]c1c([2H])c([2H])c(-c2ccc3c(c2)c2c4c5ccccc5n(-c5ccccc5)c4ccc2n3-c2ccc(-c3ccccc3)cc2)c([2H])c1[2H]]

In the composition, the first compound and the second compound may be included, for example, in a weight ratio of about 1:99 to about 99:1.

Within the above range, an appropriate weight ratio may be adjusted using the electron transport capability of the first compound and the hole transport capability of the second compound to implement bipolar characteristics and to improve the efficiency and life-span. Within the above range, for example, they may be included in a weight ratio of about 10:90 to about 90:10, about 20:80 to about 80:20, for example about 20:80 to about 70: 30, about 20:80 to about 60:40, and about 30:70 to about 60:40. As a specific example, they may be included in a weight ratio of about 40:60, about 50:50, or about 60:40.

The composition may further include one or more compounds in addition to the aforementioned first and second compounds.

The aforementioned compound for an organic optoelectronic device or the composition for an organic optoelectronic device may be implemented as a composition that further includes a dopant. The dopant may be for example a phosphorescent dopant, for example a red, green, or blue phosphorescent dopant, for example a red or green phosphorescent dopant.

A dopant is a material that emits light by being mixed in a small amount with the compound or composition for the organic optoelectronic device. In general, the dopant may be a material such as a metal complex that emits light by multiple excitation into a triplet or more. The dopant may be, for example, an inorganic, organic, or organic-inorganic compound, and may include one or two or more.

An example of the dopant may be a phosphorescent dopant, and examples of the phosphorescent dopant may include an organometallic compound including Ir, Pt, Os, Ti, Zr, Hf, Eu, Tb, Tm, Fe, Co, Ni, Ru, Rh, Pd, or a combination thereof. The phosphorescent dopant may use, e.g., a compound represented by Chemical Formula Z.

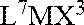

In an example embodiment of Chemical Formula Z,
M is a metal, and
L⁷ and X³ are the same as or different from each other and are ligands forming a complex with M.

In Chemical Formula Z, M may be, for example, Ir, Pt, Os, Ti, Zr, Hf, Eu, Tb, Tm, Fe, Co, Ni, Ru, Rh, Pd, or a combination thereof.

In Chemical Formula Z, L¹⁵ and X⁵ may be, for example, a bidentate ligand. Examples of the ligands represented by L⁷ and X³ may be selected from the chemical formulas of Group A.

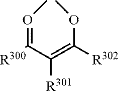

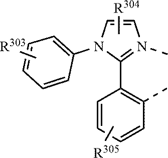

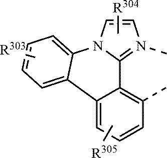

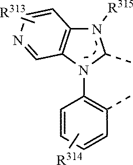

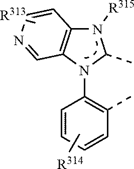

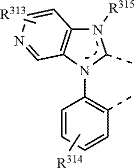

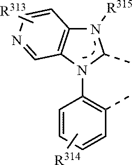

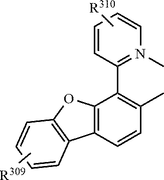

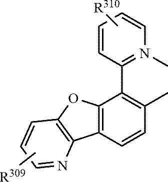

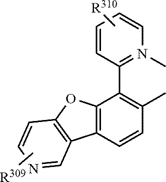

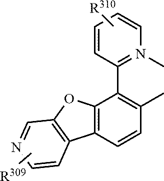

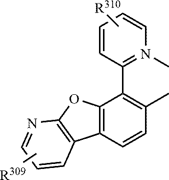

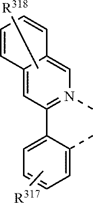

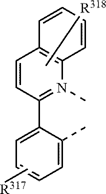

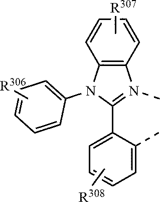

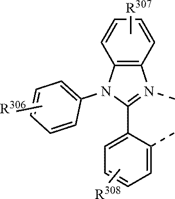

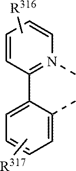

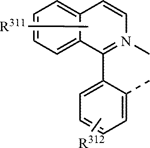

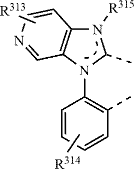

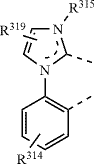

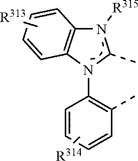

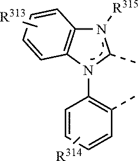

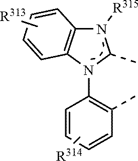

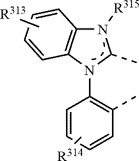

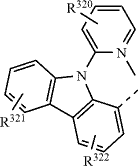

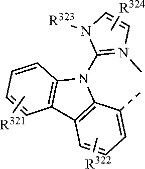

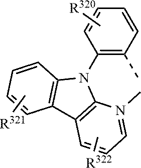

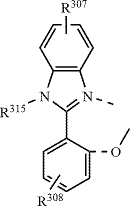

In Group A,
R³⁰⁰ to R³⁰² are each independently hydrogen, deuterium a C1 to C30 alkyl group that is substituted or unsubstituted with a halogen, a C6 to C30 aryl group that is substituted or unsubstituted with a C1 to C30 alkyl, or a halogen, and
R³⁰³ to R³²⁴ are each independently hydrogen, deuterium, halogen, a substituted or unsubstituted C1 to C30 alkyl group, a substituted or unsubstituted C1 to C30 alkoxy group, a substituted or unsubstituted C3 to C30 cycloalkyl group, a substituted or unsubstituted C2 to C30 alkenyl group, a substituted or unsubstituted C6 to C30 aryl group, a substituted or unsubstituted C1 to C30 heteroaryl group, a substituted or unsubstituted C1 to C30 amino group, a substituted or unsubstituted C6 to C30 arylamino group, SF₅, a trialkylsilyl group having a substituted or unsubstituted C1 to C30 alkyl group, a dialkylarylsilyl group having a substituted or unsubstituted C1 to C30 alkyl group and C6 to C30 aryl group, or a triarylsilyl group having a substituted or unsubstituted C6 to C30 aryl group.

As an example of the composition including a dopant, a dopant represented by Chemical Formula I may be included.

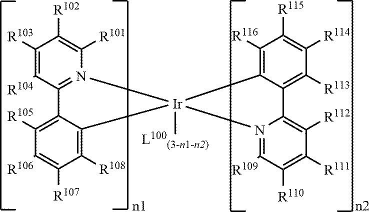

In an example embodiment of Chemical Formula I,
R¹⁰¹ to R¹¹⁶ are each independently hydrogen, deuterium, a substituted or unsubstituted C1 to C10 alkyl group, a substituted or unsubstituted C6 to C20 aryl group, or -SiR¹³²R¹³³R¹³⁴,
R¹³² to R¹³⁴ are each independently a C1 to C6 alkyl group,
at least one of R¹⁰¹ to R¹¹⁶ is a functional group represented by Chemical Formula I-1,
L¹⁰⁰ is a bidentate ligand of a monovalent anion, and is a ligand that coordinates to iridium through a lone pair of carbons or heteroatoms, and
n1 and n2 are each independently any one of integers of 0 to 3 and n1 + n2 is any one of integers of 1 to 3,

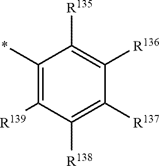

wherein, in Chemical Formula I-1,
R¹³⁵ to R¹³⁹ are each independently hydrogen, deuterium, a substituted or unsubstituted C1 to C10 alkyl group, a substituted or unsubstituted C6 to C20 aryl group, or -SiR¹³²R¹³³R¹³⁴, and
indicates a portion linked to a carbon atom.

As an example of the composition including a dopant, a dopant represented by Chemical Formula Z-1 may be included.

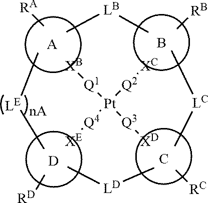

In an example embodiment of Chemical Formula Z-1,
rings A, B, C, and D are each independently a 5-membered or 6-membered carbocyclic or heterocyclic ring;
R^{A}, R^{B}, R^{C}, and R^{D} are each independently mono-, di-, tri-, or tetra-substituted, or unsubstituted;
L^{B}, L^{C}, and L^{D} are each independently selected from a direct bond, BR, NR, PR, O, S, Se, C=O, S=O, SO₂, CRR′, SiRR′, GeRR′, and a combination thereof;
when nA is 1, L^{E} is selected from a direct bond, BR, NR, PR, O, S, Se, C=O, S=O, SO₂, CRR′, SiRR′, GeRR′, and a combination thereof; when nA is 0, L^{E} does not exist; and
R^{A}, R^{B}, R^{C}, R^{D}, R, and R′ are each independently selected from hydrogen, deuterium, a halogen, an alkyl group, a cycloalkyl group, a heteroalkyl group, an arylalkyl group, an alkoxy group, an aryloxy group, an amino group, a silyl group, an alkenyl group, a cycloalkenyl group, a heteroalkenyl group, an alkynyl group, an aryl group, a heteroaryl group, an acyl group, a carbonyl group, a carboxylic acid group, an ester group, a nitrile group, an isonitrile group, a sulfanyl group, a sulfinyl group, a sulfonyl group, a phosphino group, and a combination thereof; any adjacent R^{A}, R^{B}, R^{C}, R^{D}, R, and R′ are optionally linked to each other to provide a ring; X^{B}, X^{C}, X^{D}, and X^{E} are each independently selected from carbon and nitrogen; and Q¹, Q², Q³, and Q⁴ each represent oxygen or a direct bond.

The dopant according to an example embodiment may be a platinum complex, and may be, for example, represented by Chemical Formula II.

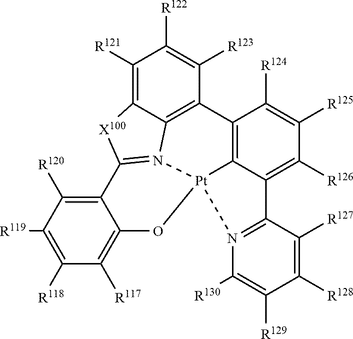

In Chemical Formula II,
X¹⁰⁰ is selected from O, S, and NR¹³¹,
R¹¹⁷ to R¹³¹ are each independently hydrogen, deuterium, a substituted or unsubstituted C1 to C10 alkyl group, a substituted or unsubstituted C6 to C20 aryl group, or - SiR¹³²R¹³³R¹³⁴,
R¹³² to R¹³⁴ are each independently a C1 to C6 alkyl group, and
at least one of R¹¹⁷ to R¹³¹ is -SiR¹³²R¹³³R¹³⁴ or a tert-butyl group.

Hereinafter, an organic optoelectronic device including the aforementioned compound for the organic optoelectronic device or composition for the organic optoelectronic device is described.

The organic optoelectronic device may be any device to convert electrical energy into photoenergy and vice versa, and may be, for example an organic photoelectric device, an organic light emitting diode, an organic solar cell, and an organic photoconductor drum.

Herein, an organic light emitting diode as one example of an organic optoelectronic device is described referring to drawing.

The FIGURE is a cross-sectional view illustrating an organic light emitting diode according to an example embodiment.

Referring to the FIGURE, an organic light emitting diode 100 according to an example embodiment includes an anode 120 and a cathode 110 facing each other and an organic layer 105 disposed between the anode 120 and cathode 110.

The anode 120 may be made of a conductor having a large work function to help hole injection, and may be for example a metal, a metal oxide and/or a conductive polymer. The anode 120 may be, for example a metal such as nickel, platinum, vanadium, chromium, copper, zinc, gold, and the like or an alloy thereof; a metal oxide such as zinc oxide, indium oxide, indium tin oxide (ITO), indium zinc oxide (IZO), and the like; a combination of a metal and an oxide such as ZnO and Al or SnO₂ and Sb; or a conductive polymer such as poly(3-methylthiophene), poly(3,4-(ethylene-1,2-dioxy)thiophene) (PEDOT), polypyrrole, and polyaniline.

The cathode 110 may be made of a conductor having a small work function to help electron injection, and may be for example a metal, a metal oxide, and/or a conductive polymer. The cathode 110 may be for example a metal such as magnesium, calcium, sodium, potassium, titanium, indium, yttrium, lithium, gadolinium, aluminum silver, tin, lead, cesium, barium, and the like, or an alloy thereof; or a multi-layer structure material such as LiF/Al, LiO₂/Al, LiF/Ca, and BaF₂/Ca.

The organic layer 105 may include the aforementioned compound for the organic optoelectronic device or composition for the organic optoelectronic device.

The organic layer 105 may include, for example, the light emitting layer 130, and the light emitting layer 130 may include, for example, the aforementioned compound for the organic optoelectronic device or composition for the organic optoelectronic device.

The composition for the organic optoelectronic device further including a dopant may be, for example, a green light-emitting composition.

The light emitting layer 130 may include, for example, the aforementioned compound for the organic optoelectronic device or composition for the organic optoelectronic device as a phosphorescent host.

The organic layer may further include a charge transport region in addition to the light emitting layer.

The charge transport region may be, for example, the hole transport region 140.

The hole transport region 140 may further increase hole injection and/or hole mobility between the anode 120 and the light emitting layer 130 and block electrons. Specifically, the hole transport region 140 may include a hole transport layer between the anode 120 and the light emitting layer 130, and a hole transport auxiliary layer between the light emitting layer 130 and the hole transport layer, and at least one of the compounds of Group B may be included in at least one of the hole transport layer and the hole transport auxiliary layer.

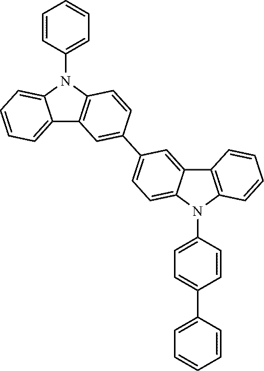 [SMILES: c1ccc(-c2ccc(-n3c4ccccc4c4cc(-c5ccc6c(c5)c5ccccc5n6-c5ccccc5)ccc43)cc2)cc1]

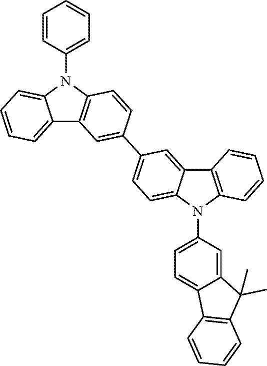 [SMILES: CC1(C)c2ccccc2-c2ccc(-n3c4ccccc4c4cc(-c5ccc6c(c5)c5ccccc5n6-c5ccccc5)ccc43)cc21]

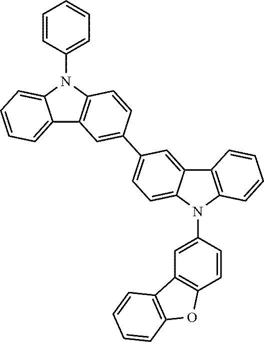 [SMILES: c1ccc(-n2c3ccccc3c3cc(-c4ccc5c(c4)c4ccccc4n5-c4ccc5oc6ccccc6c5c4)ccc32)cc1]

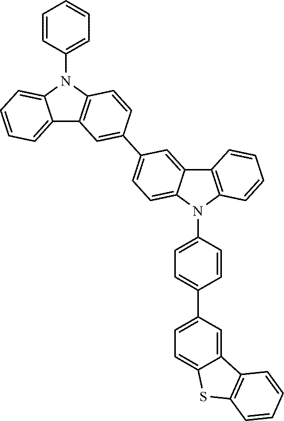 [SMILES: c1ccc(-n2c3ccccc3c3cc(-c4ccc5c(c4)c4ccccc4n5-c4ccc(-c5ccc6sc7ccccc7c6c5)cc4)ccc32)cc1]

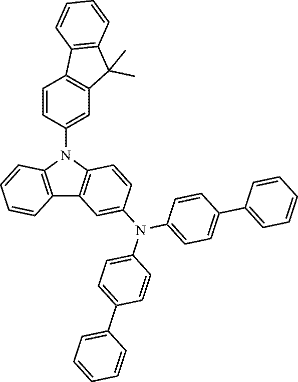 [SMILES: CC1(C)c2ccccc2-c2ccc(-n3c4ccccc4c4cc(N(c5ccc(-c6ccccc6)cc5)c5ccc(-c6ccccc6)cc5)ccc43)cc21]

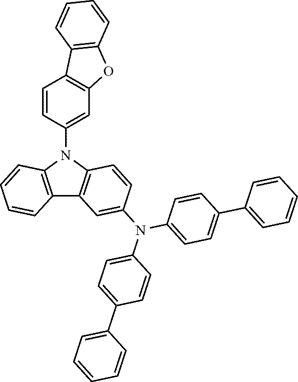 [SMILES: c1ccc(-c2ccc(N(c3ccc(-c4ccccc4)cc3)c3ccc4c(c3)c3ccccc3n4-c3ccc4c(c3)oc3ccccc34)cc2)cc1]

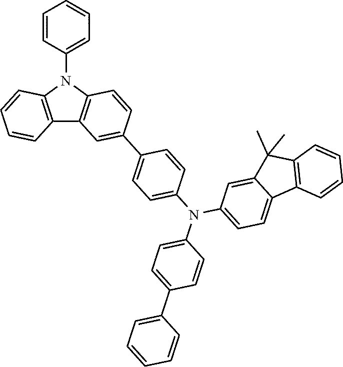 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21]

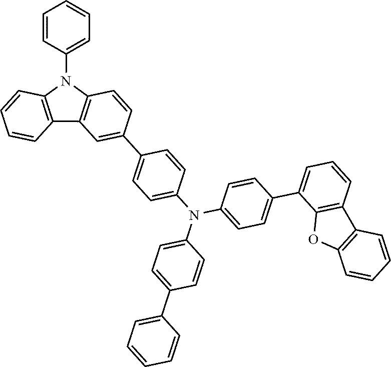 [SMILES: c1ccc(-c2ccc(N(c3ccc(-c4ccc5c(c4)c4ccccc4n5-c4ccccc4)cc3)c3ccc(-c4cccc5c4oc4ccccc45)cc3)cc2)cc1]

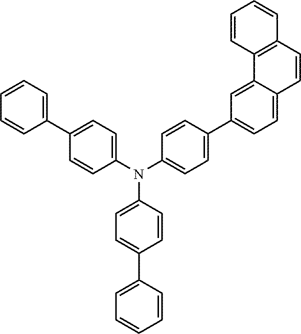 [SMILES: c1ccc(-c2ccc(N(c3ccc(-c4ccccc4)cc3)c3ccc(-c4ccc5ccc6ccccc6c5c4)cc3)cc2)cc1]

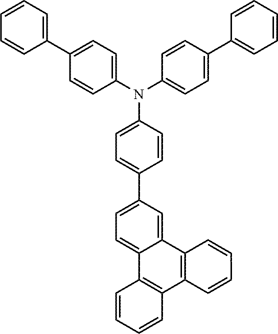 [SMILES: c1ccc(-c2ccc(N(c3ccc(-c4ccccc4)cc3)c3ccc(-c4ccc5c6ccccc6c6ccccc6c5c4)cc3)cc2)cc1]

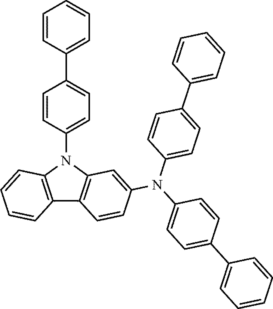 [SMILES: c1ccc(-c2ccc(N(c3ccc(-c4ccccc4)cc3)c3ccc4c5ccccc5n(-c5ccc(-c6ccccc6)cc5)c4c3)cc2)cc1]

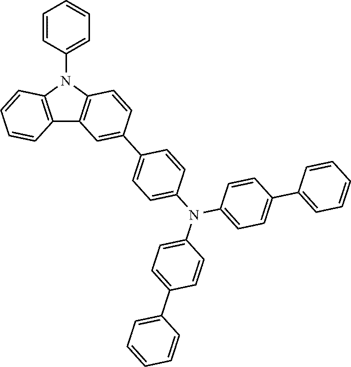 [SMILES: c1ccc(-c2ccc(N(c3ccc(-c4ccccc4)cc3)c3ccc(-c4ccc5c(c4)c4ccccc4n5-c4ccccc4)cc3)cc2)cc1]

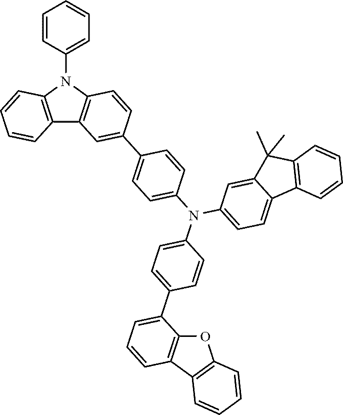 [SMILES: CC1(C)c2ccccc2-c2ccc(N(c3ccc(-c4ccc5c(c4)c4ccccc4n5-c4ccccc4)cc3)c3ccc(-c4cccc5c4oc4ccccc45)cc3)cc21]

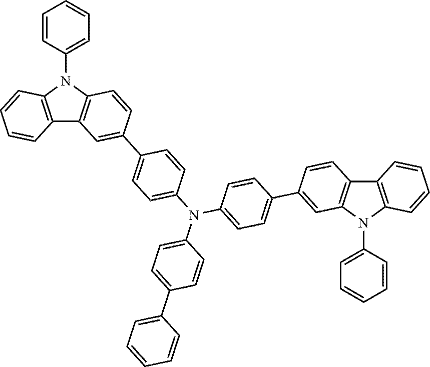 [SMILES: c1ccc(-c2ccc(N(c3ccc(-c4ccc5c(c4)c4ccccc4n5-c4ccccc4)cc3)c3ccc(-c4ccc5c6ccccc6n(-c6ccccc6)c5c4)cc3)cc2)cc1]

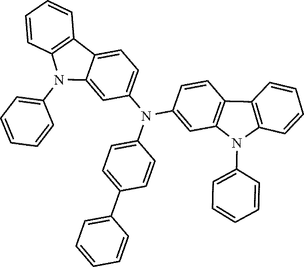 [SMILES: c1ccc(-c2ccc(N(c3ccc4c5ccccc5n(-c5ccccc5)c4c3)c3ccc4c5ccccc5n(-c5ccccc5)c4c3)cc2)cc1]

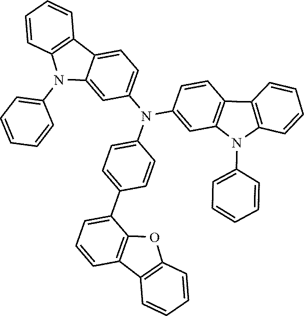 [SMILES: c1ccc(-n2c3ccccc3c3ccc(N(c4ccc(-c5cccc6c5oc5ccccc56)cc4)c4ccc5c6ccccc6n(-c6ccccc6)c5c4)cc32)cc1]

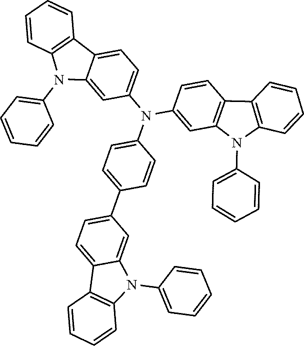 [SMILES: c1ccc(-n2c3ccccc3c3ccc(-c4ccc(N(c5ccc6c7ccccc7n(-c7ccccc7)c6c5)c5ccc6c7ccccc7n(-c7ccccc7)c6c5)cc4)cc32)cc1]

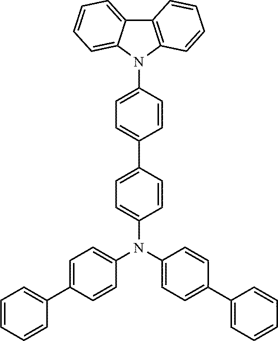 [SMILES: c1ccc(-c2ccc(N(c3ccc(-c4ccccc4)cc3)c3ccc(-c4ccc(-n5c6ccccc6c6ccccc65)cc4)cc3)cc2)cc1]

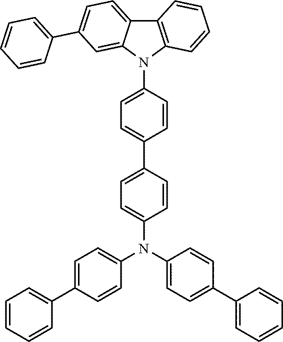 [SMILES: c1ccc(-c2ccc(N(c3ccc(-c4ccccc4)cc3)c3ccc(-c4ccc(-n5c6ccccc6c6ccc(-c7ccccc7)cc65)cc4)cc3)cc2)cc1]

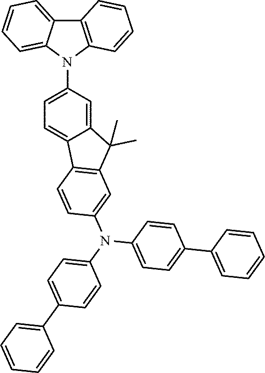 [SMILES: CC1(C)c2cc(N(c3ccc(-c4ccccc4)cc3)c3ccc(-c4ccccc4)cc3)ccc2-c2ccc(-n3c4ccccc4c4ccccc43)cc21]

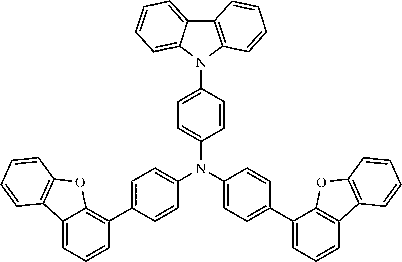 [SMILES: c1ccc2c(c1)oc1c(-c3ccc(N(c4ccc(-c5cccc6c5oc5ccccc56)cc4)c4ccc(-n5c6ccccc6c6ccccc65)cc4)cc3)cccc12]

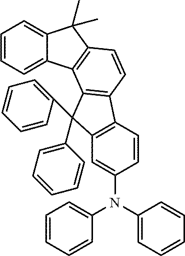 [SMILES: CC1(C)c2ccccc2-c2c1ccc1c2C(c2ccccc2)(c2ccccc2)c2cc(N(c3ccccc3)c3ccccc3)ccc2-1]

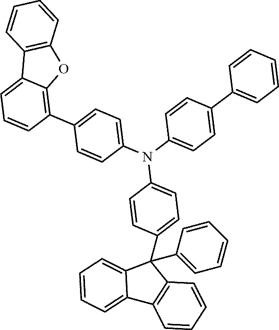 [SMILES: c1ccc(-c2ccc(N(c3ccc(-c4cccc5c4oc4ccccc45)cc3)c3ccc(C4(c5ccccc5)c5ccccc5-c5ccccc54)cc3)cc2)cc1]

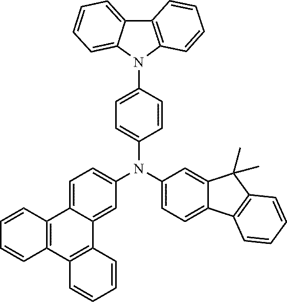 [SMILES: CC1(C)c2ccccc2-c2ccc(N(c3ccc(-n4c5ccccc5c5ccccc54)cc3)c3ccc4c5ccccc5c5ccccc5c4c3)cc21]

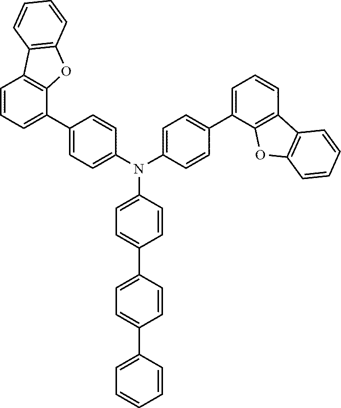 [SMILES: c1ccc(-c2ccc(-c3ccc(N(c4ccc(-c5cccc6c5oc5ccccc56)cc4)c4ccc(-c5cccc6c5oc5ccccc56)cc4)cc3)cc2)cc1]

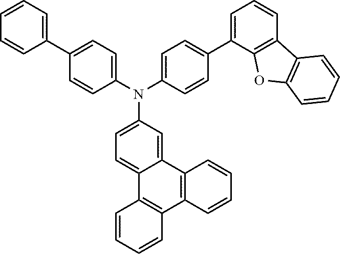 [SMILES: c1ccc(-c2ccc(N(c3ccc(-c4cccc5c4oc4ccccc45)cc3)c3ccc4c5ccccc5c5ccccc5c4c3)cc2)cc1]

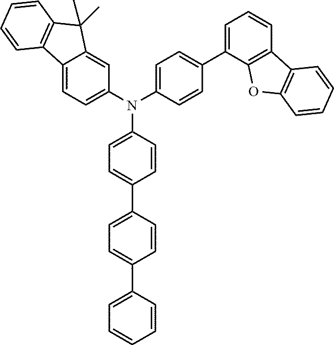 [SMILES: CC1(C)c2ccccc2-c2ccc(N(c3ccc(-c4ccc(-c5ccccc5)cc4)cc3)c3ccc(-c4cccc5c4oc4ccccc45)cc3)cc21]

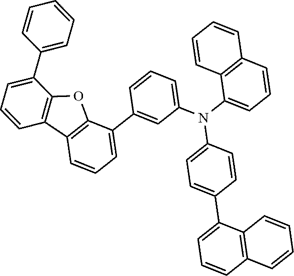 [SMILES: c1ccc(-c2cccc3c2oc2c(-c4cccc(N(c5ccc(-c6cccc7ccccc67)cc5)c5cccc6ccccc56)c4)cccc23)cc1]

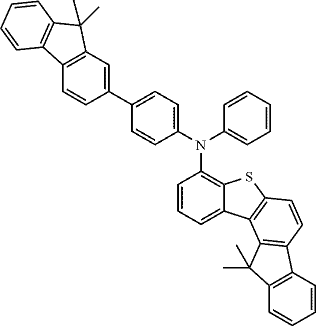 [SMILES: CC1(C)c2ccccc2-c2ccc(-c3ccc(N(c4ccccc4)c4cccc5c4sc4ccc6c(c45)C(C)(C)c4ccccc4-6)cc3)cc21]

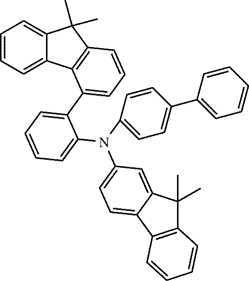 [SMILES: CC1(C)c2ccccc2-c2ccc(N(c3ccc(-c4ccccc4)cc3)c3ccccc3-c3cccc4c3-c3ccccc3C4(C)C)cc21]

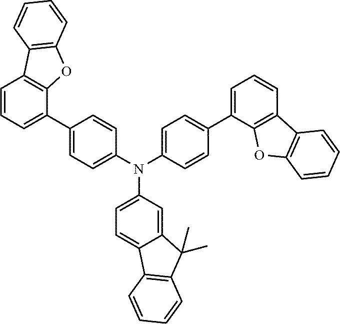 [SMILES: CC1(C)c2ccccc2-c2ccc(N(c3ccc(-c4cccc5c4oc4ccccc45)cc3)c3ccc(-c4cccc5c4oc4ccccc45)cc3)cc21]

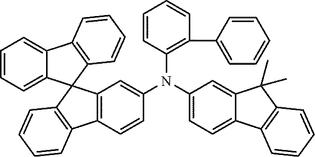 [SMILES: CC1(C)c2ccccc2-c2ccc(N(c3ccc4c(c3)C3(c5ccccc5-c5ccccc53)c3ccccc3-4)c3ccccc3-c3ccccc3)cc21]

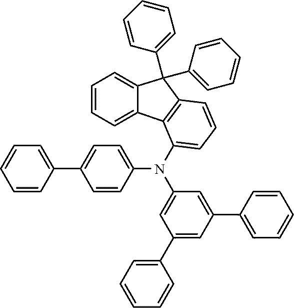 [SMILES: c1ccc(-c2ccc(N(c3cc(-c4ccccc4)cc(-c4ccccc4)c3)c3cccc4c3-c3ccccc3C4(c3ccccc3)c3ccccc3)cc2)cc1]

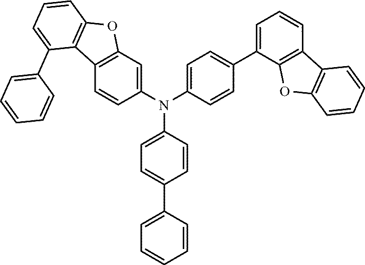 [SMILES: c1ccc(-c2ccc(N(c3ccc(-c4cccc5c4oc4ccccc45)cc3)c3ccc4c(c3)oc3cccc(-c5ccccc5)c34)cc2)cc1]

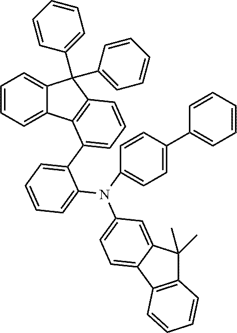 [SMILES: CC1(C)c2ccccc2-c2ccc(N(c3ccc(-c4ccccc4)cc3)c3ccccc3-c3cccc4c3-c3ccccc3C4(c3ccccc3)c3ccccc3)cc21]

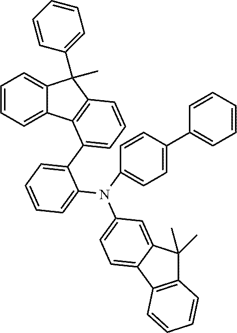 [SMILES: CC1(C)c2ccccc2-c2ccc(N(c3ccc(-c4ccccc4)cc3)c3ccccc3-c3cccc4c3-c3ccccc3C4(C)c3ccccc3)cc21]

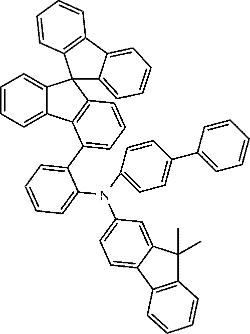 [SMILES: CC1(C)c2ccccc2-c2ccc(N(c3ccc(-c4ccccc4)cc3)c3ccccc3-c3cccc4c3-c3ccccc3C43c4ccccc4-c4ccccc43)cc21]

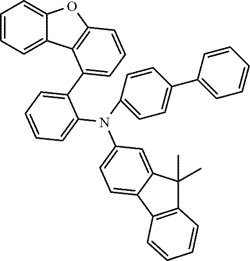 [SMILES: CC1(C)c2ccccc2-c2ccc(N(c3ccc(-c4ccccc4)cc3)c3ccccc3-c3cccc4oc5ccccc5c34)cc21]

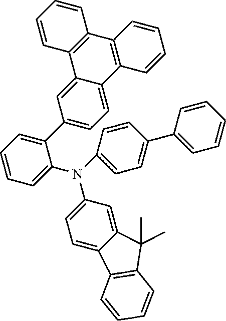 [SMILES: CC1(C)c2ccccc2-c2ccc(N(c3ccc(-c4ccccc4)cc3)c3ccccc3-c3ccc4c5ccccc5c5ccccc5c4c3)cc21]

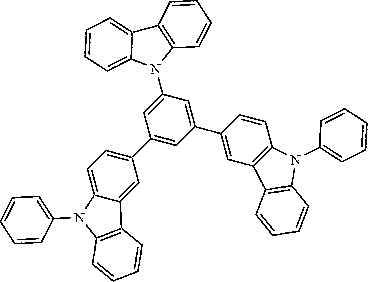 [SMILES: c1ccc(-n2c3ccccc3c3cc(-c4cc(-c5ccc6c(c5)c5ccccc5n6-c5ccccc5)cc(-n5c6ccccc6c6ccccc65)c4)ccc32)cc1]

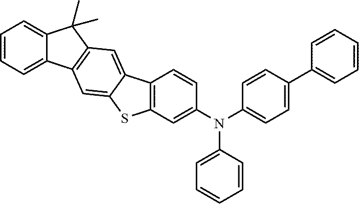 [SMILES: CC1(C)c2ccccc2-c2cc3sc4cc(N(c5ccccc5)c5ccc(-c6ccccc6)cc5)ccc4c3cc21]

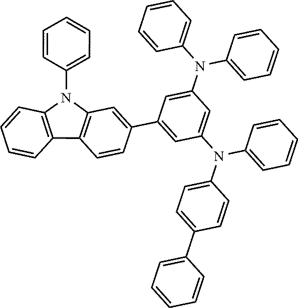 [SMILES: c1ccc(-c2ccc(N(c3ccccc3)c3cc(-c4ccc5c6ccccc6n(-c6ccccc6)c5c4)cc(N(c4ccccc4)c4ccccc4)c3)cc2)cc1]

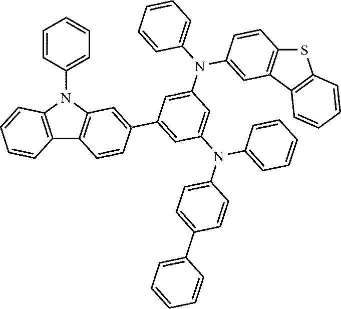 [SMILES: c1ccc(-c2ccc(N(c3ccccc3)c3cc(-c4ccc5c6ccccc6n(-c6ccccc6)c5c4)cc(N(c4ccccc4)c4ccc5sc6ccccc6c5c4)c3)cc2)cc1]

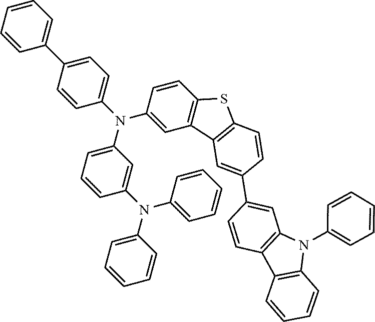 [SMILES: c1ccc(-c2ccc(N(c3cccc(N(c4ccccc4)c4ccccc4)c3)c3ccc4sc5ccc(-c6ccc7c8ccccc8n(-c8ccccc8)c7c6)cc5c4c3)cc2)cc1]

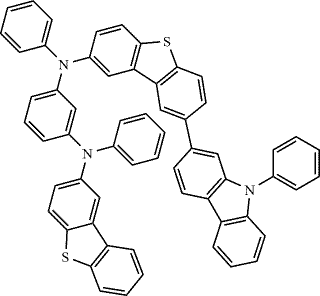 [SMILES: c1ccc(N(c2cccc(N(c3ccccc3)c3ccc4sc5ccc(-c6ccc7c8ccccc8n(-c8ccccc8)c7c6)cc5c4c3)c2)c2ccc3sc4ccccc4c3c2)cc1]

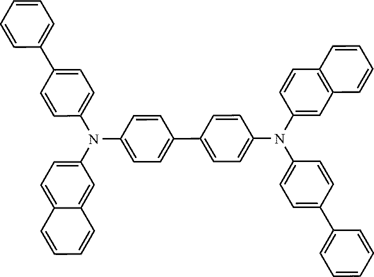 [SMILES: c1ccc(-c2ccc(N(c3ccc(-c4ccc(N(c5ccc(-c6ccccc6)cc5)c5ccc6ccccc6c5)cc4)cc3)c3ccc4ccccc4c3)cc2)cc1]

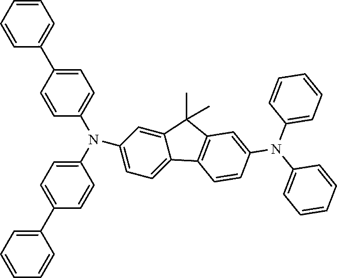 [SMILES: CC1(C)c2cc(N(c3ccccc3)c3ccccc3)ccc2-c2ccc(N(c3ccc(-c4ccccc4)cc3)c3ccc(-c4ccccc4)cc3)cc21]

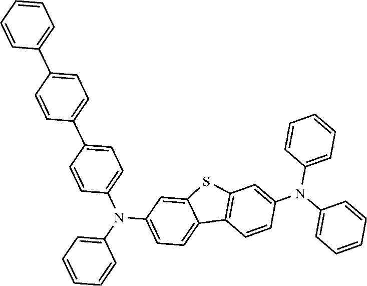 [SMILES: c1ccc(-c2ccc(-c3ccc(N(c4ccccc4)c4ccc5c(c4)sc4cc(N(c6ccccc6)c6ccccc6)ccc45)cc3)cc2)cc1]

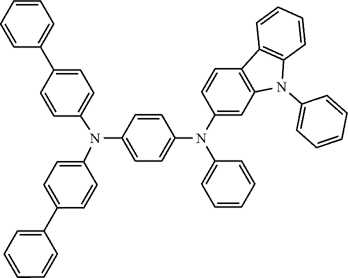 [SMILES: c1ccc(-c2ccc(N(c3ccc(-c4ccccc4)cc3)c3ccc(N(c4ccccc4)c4ccc5c6ccccc6n(-c6ccccc6)c5c4)cc3)cc2)cc1]

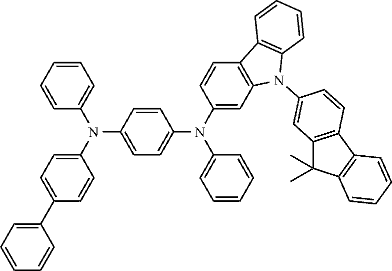 [SMILES: CC1(C)c2ccccc2-c2ccc(-n3c4ccccc4c4ccc(N(c5ccccc5)c5ccc(N(c6ccccc6)c6ccc(-c7ccccc7)cc6)cc5)cc43)cc21]

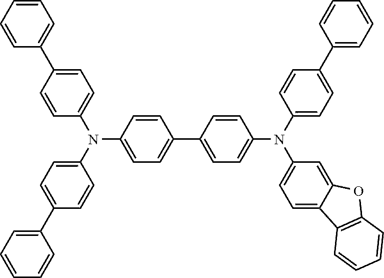 [SMILES: c1ccc(-c2ccc(N(c3ccc(-c4ccccc4)cc3)c3ccc(-c4ccc(N(c5ccc(-c6ccccc6)cc5)c5ccc6c(c5)oc5ccccc56)cc4)cc3)cc2)cc1]

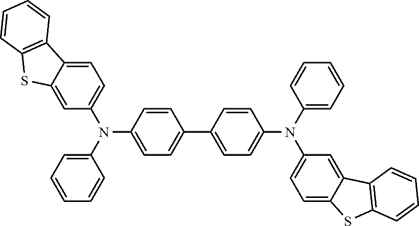 [SMILES: c1ccc(N(c2ccc(-c3ccc(N(c4ccccc4)c4ccc5sc6ccccc6c5c4)cc3)cc2)c2ccc3c(c2)sc2ccccc23)cc1]

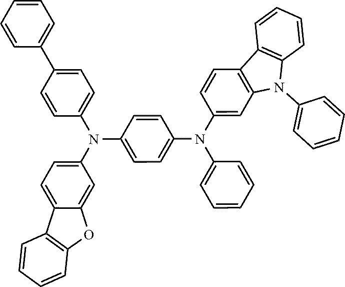 [SMILES: c1ccc(-c2ccc(N(c3ccc(N(c4ccccc4)c4ccc5c6ccccc6n(-c6ccccc6)c5c4)cc3)c3ccc4c(c3)oc3ccccc34)cc2)cc1]

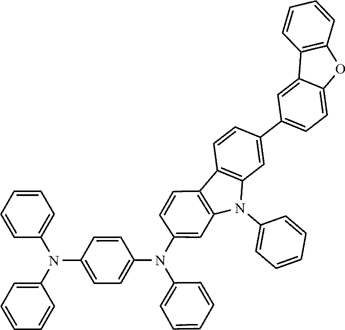 [SMILES: c1ccc(N(c2ccccc2)c2ccc(N(c3ccccc3)c3ccc4c5ccc(-c6ccc7oc8ccccc8c7c6)cc5n(-c5ccccc5)c4c3)cc2)cc1]

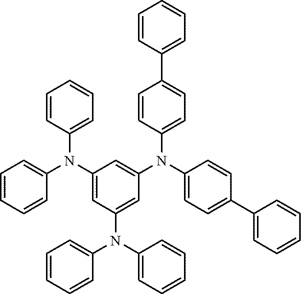 [SMILES: c1ccc(-c2ccc(N(c3ccc(-c4ccccc4)cc3)c3cc(N(c4ccccc4)c4ccccc4)cc(N(c4ccccc4)c4ccccc4)c3)cc2)cc1]

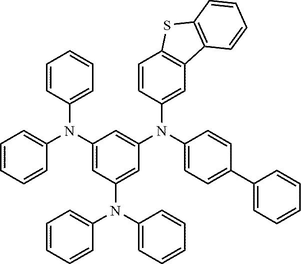 [SMILES: c1ccc(-c2ccc(N(c3cc(N(c4ccccc4)c4ccccc4)cc(N(c4ccccc4)c4ccccc4)c3)c3ccc4sc5ccccc5c4c3)cc2)cc1]

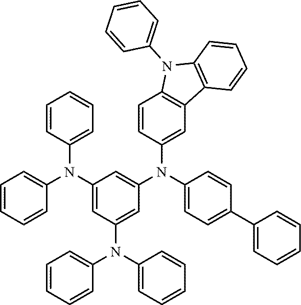 [SMILES: c1ccc(-c2ccc(N(c3cc(N(c4ccccc4)c4ccccc4)cc(N(c4ccccc4)c4ccccc4)c3)c3ccc4c(c3)c3ccccc3n4-c3ccccc3)cc2)cc1]

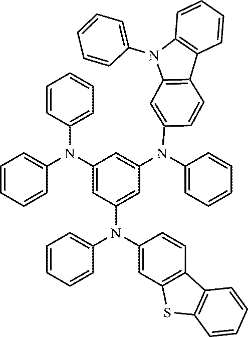 [SMILES: c1ccc(N(c2ccccc2)c2cc(N(c3ccccc3)c3ccc4c(c3)sc3ccccc34)cc(N(c3ccccc3)c3ccc4c5ccccc5n(-c5ccccc5)c4c3)c2)cc1]

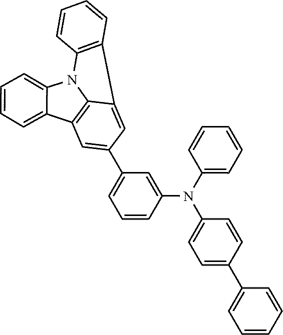 [SMILES: c1ccc(-c2ccc(N(c3ccccc3)c3cccc(-c4cc5c6ccccc6n6c7ccccc7c(c4)c56)c3)cc2)cc1]

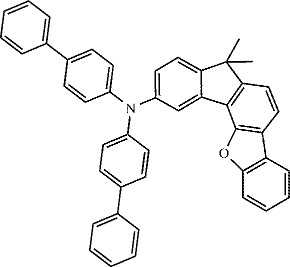 [SMILES: CC1(C)c2ccc(N(c3ccc(-c4ccccc4)cc3)c3ccc(-c4ccccc4)cc3)cc2-c2c1ccc1c2oc2ccccc21]

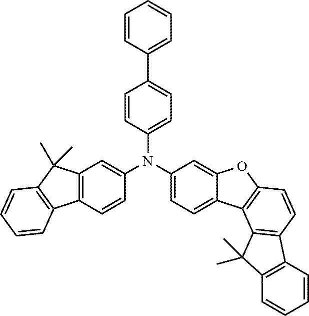 [SMILES: CC1(C)c2ccccc2-c2ccc(N(c3ccc(-c4ccccc4)cc3)c3ccc4c(c3)oc3ccc5c(c34)C(C)(C)c3ccccc3-5)cc21]

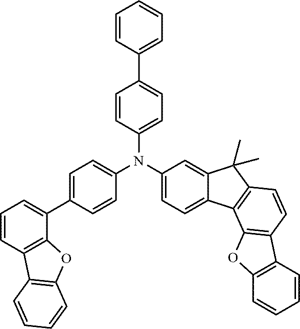 [SMILES: CC1(C)c2cc(N(c3ccc(-c4ccccc4)cc3)c3ccc(-c4cccc5c4oc4ccccc45)cc3)ccc2-c2c1ccc1c2oc2ccccc21]

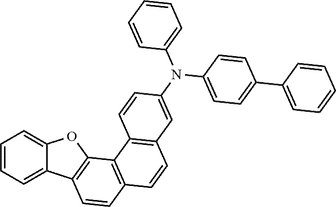 [SMILES: c1ccc(-c2ccc(N(c3ccccc3)c3ccc4c(ccc5ccc6c7ccccc7oc6c54)c3)cc2)cc1]

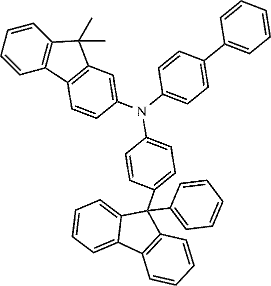 [SMILES: CC1(C)c2ccccc2-c2ccc(N(c3ccc(-c4ccccc4)cc3)c3ccc(C4(c5ccccc5)c5ccccc5-c5ccccc54)cc3)cc21]

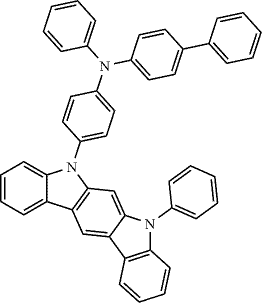 [SMILES: c1ccc(-c2ccc(N(c3ccccc3)c3ccc(-n4c5ccccc5c5cc6c7ccccc7n(-c7ccccc7)c6cc54)cc3)cc2)cc1]

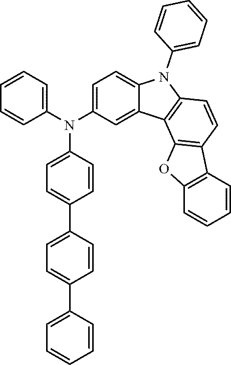 [SMILES: c1ccc(-c2ccc(-c3ccc(N(c4ccccc4)c4ccc5c(c4)c4c6oc7ccccc7c6ccc4n5-c4ccccc4)cc3)cc2)cc1]

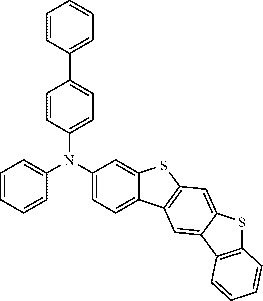 [SMILES: c1ccc(-c2ccc(N(c3ccccc3)c3ccc4c(c3)sc3cc5sc6ccccc6c5cc34)cc2)cc1]

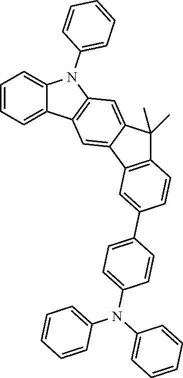 [SMILES: CC1(C)c2ccc(-c3ccc(N(c4ccccc4)c4ccccc4)cc3)cc2-c2cc3c4ccccc4n(-c4ccccc4)c3cc21]

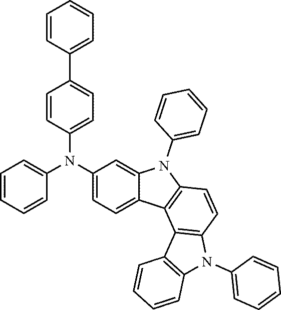 [SMILES: c1ccc(-c2ccc(N(c3ccccc3)c3ccc4c5c6c7ccccc7n(-c7ccccc7)c6ccc5n(-c5ccccc5)c4c3)cc2)cc1]

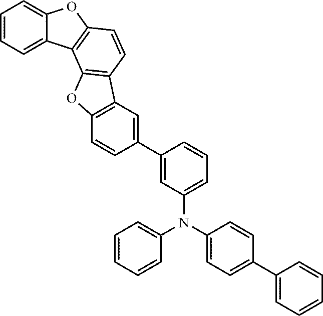 [SMILES: c1ccc(-c2ccc(N(c3ccccc3)c3cccc(-c4ccc5oc6c(ccc7oc8ccccc8c76)c5c4)c3)cc2)cc1]

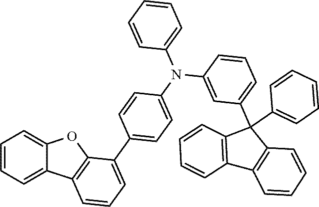 [SMILES: c1ccc(N(c2ccc(-c3cccc4c3oc3ccccc34)cc2)c2cccc(C3(c4ccccc4)c4ccccc4-c4ccccc43)c2)cc1]

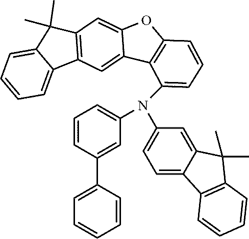 [SMILES: CC1(C)c2ccccc2-c2ccc(N(c3cccc(-c4ccccc4)c3)c3cccc4oc5cc6c(cc5c34)-c3ccccc3C6(C)C)cc21]

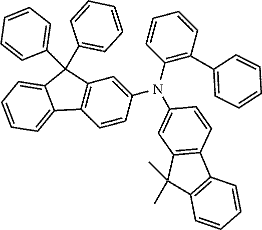 [SMILES: CC1(C)c2ccccc2-c2ccc(N(c3ccc4c(c3)C(c3ccccc3)(c3ccccc3)c3ccccc3-4)c3ccccc3-c3ccccc3)cc21]

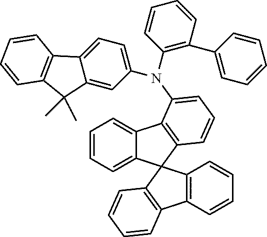 [SMILES: CC1(C)c2ccccc2-c2ccc(N(c3ccccc3-c3ccccc3)c3cccc4c3-c3ccccc3C43c4ccccc4-c4ccccc43)cc21]

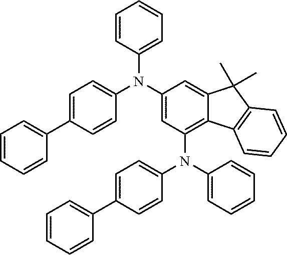 [SMILES: CC1(C)c2ccccc2-c2c(N(c3ccccc3)c3ccc(-c4ccccc4)cc3)cc(N(c3ccccc3)c3ccc(-c4ccccc4)cc3)cc21]

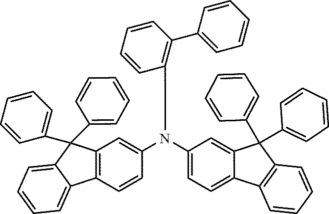 [SMILES: c1ccc(-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1]

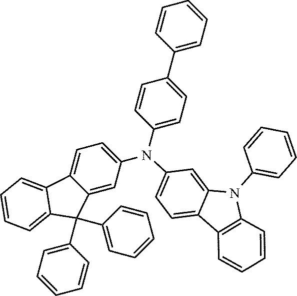 [SMILES: c1ccc(-c2ccc(N(c3ccc4c(c3)C(c3ccccc3)(c3ccccc3)c3ccccc3-4)c3ccc4c5ccccc5n(-c5ccccc5)c4c3)cc2)cc1]

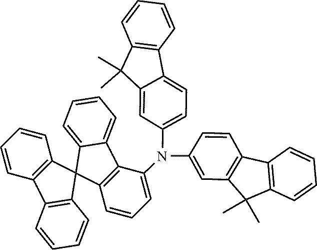 [SMILES: CC1(C)c2ccccc2-c2ccc(N(c3ccc4c(c3)C(C)(C)c3ccccc3-4)c3cccc4c3-c3ccccc3C43c4ccccc4-c4ccccc43)cc21]

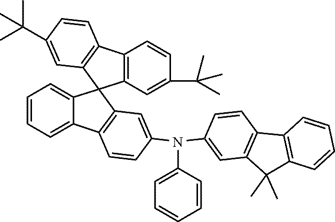 [SMILES: CC(C)(C)c1ccc2c(c1)C1(c3ccccc3-c3ccc(N(c4ccccc4)c4ccc5c(c4)C(C)(C)c4ccccc4-5)cc31)c1cc(C(C)(C)C)ccc1-2]

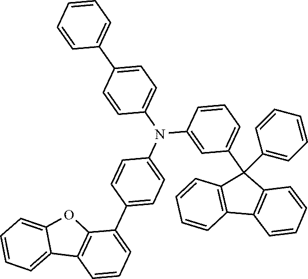 [SMILES: c1ccc(-c2ccc(N(c3ccc(-c4cccc5c4oc4ccccc45)cc3)c3cccc(C4(c5ccccc5)c5ccccc5-c5ccccc54)c3)cc2)cc1]

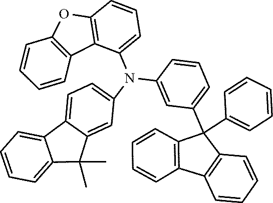 [SMILES: CC1(C)c2ccccc2-c2ccc(N(c3cccc(C4(c5ccccc5)c5ccccc5-c5ccccc54)c3)c3cccc4oc5ccccc5c34)cc21]

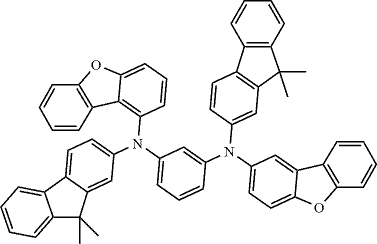 [SMILES: CC1(C)c2ccccc2-c2ccc(N(c3cccc(N(c4ccc5c(c4)C(C)(C)c4ccccc4-5)c4cccc5oc6ccccc6c45)c3)c3ccc4oc5ccccc5c4c3)cc21]

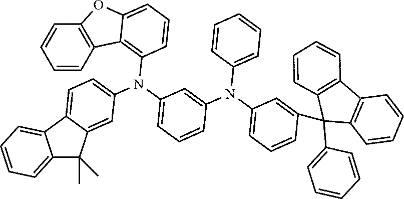 [SMILES: CC1(C)c2ccccc2-c2ccc(N(c3cccc(N(c4ccccc4)c4cccc(C5(c6ccccc6)c6ccccc6-c6ccccc65)c4)c3)c3cccc4oc5ccccc5c34)cc21]

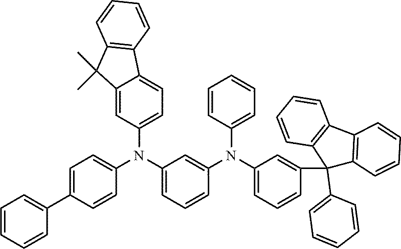 [SMILES: CC1(C)c2ccccc2-c2ccc(N(c3ccc(-c4ccccc4)cc3)c3cccc(N(c4ccccc4)c4cccc(C5(c6ccccc6)c6ccccc6-c6ccccc65)c4)c3)cc21]

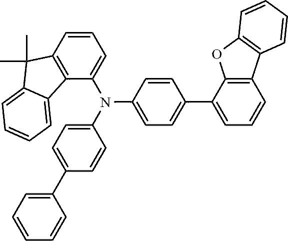 [SMILES: CC1(C)c2ccccc2-c2c(N(c3ccc(-c4ccccc4)cc3)c3ccc(-c4cccc5c4oc4ccccc45)cc3)cccc21]

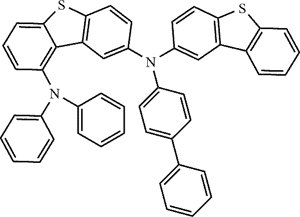 [SMILES: c1ccc(-c2ccc(N(c3ccc4sc5ccccc5c4c3)c3ccc4sc5cccc(N(c6ccccc6)c6ccccc6)c5c4c3)cc2)cc1]

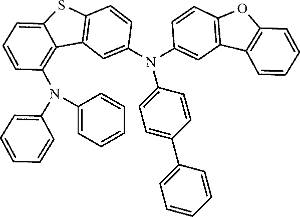 [SMILES: c1ccc(-c2ccc(N(c3ccc4oc5ccccc5c4c3)c3ccc4sc5cccc(N(c6ccccc6)c6ccccc6)c5c4c3)cc2)cc1]

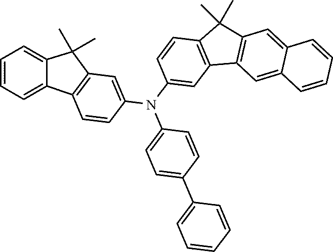 [SMILES: CC1(C)c2ccccc2-c2ccc(N(c3ccc(-c4ccccc4)cc3)c3ccc4c(c3)-c3cc5ccccc5cc3C4(C)C)cc21]

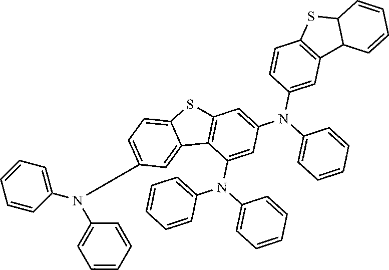 [SMILES: C1=CC2Sc3ccc(N(c4ccccc4)c4cc(N(c5ccccc5)c5ccccc5)c5c(c4)sc4ccc(N(c6ccccc6)c6ccccc6)cc45)cc3C2C=C1]

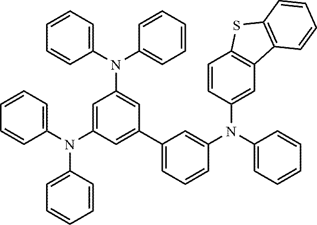 [SMILES: c1ccc(N(c2ccccc2)c2cc(-c3cccc(N(c4ccccc4)c4ccc5sc6ccccc6c5c4)c3)cc(N(c3ccccc3)c3ccccc3)c2)cc1]

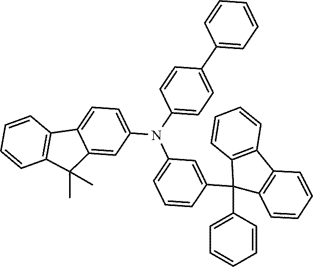 [SMILES: CC1(C)c2ccccc2-c2ccc(N(c3ccc(-c4ccccc4)cc3)c3cccc(C4(c5ccccc5)c5ccccc5-c5ccccc54)c3)cc21]

In the hole transport region 140, compounds disclosed in U.S. 5,061,569, JP 1993-009471 A, WO 1995-009147 A1, JP 1995-126615 A, JP 1998-095973 A, and the like, and compounds similar thereto, may be used in addition to the compounds.

Also, the charge transport region may be, for example, the electron transport region 150.

The electron transport region 150 may further increase electron injection and/or electron mobility between the cathode 110 and the light emitting layer 130 and block holes.

Specifically, the electron transport region 150 may include an electron transport layer between the cathode 110 and the light emitting layer 130, and an electron transport auxiliary layer between the light emitting layer 130 and the electron transport layer and at least one of the compounds of Group C may be included in at least one of the electron transport layer and the electron transport auxiliary layer.

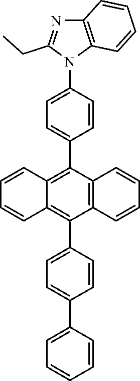 [SMILES: CCc1nc2ccccc2n1-c1ccc(-c2c3ccccc3c(-c3ccc(-c4ccccc4)cc3)c3ccccc23)cc1]

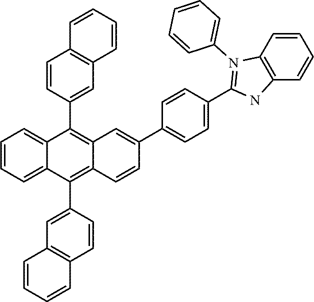

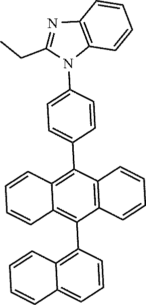 [SMILES: CCc1nc2ccccc2n1-c1ccc(-c2c3ccccc3c(-c3cccc4ccccc34)c3ccccc23)cc1]

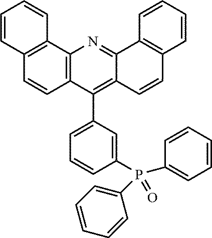 [SMILES: O=P(c1ccccc1)(c1ccccc1)c1cccc(-c2c3ccc4ccccc4c3nc3c2ccc2ccccc23)c1]

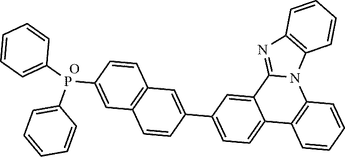

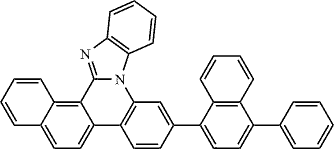 [SMILES: c1ccc(-c2ccc(-c3ccc4c5ccc6ccccc6c5c5nc6ccccc6n5c4c3)c3ccccc23)cc1]

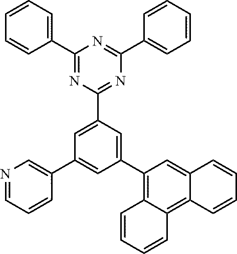 [SMILES: c1ccc(-c2nc(-c3ccccc3)nc(-c3cc(-c4cccnc4)cc(-c4cc5ccccc5c5ccccc45)c3)n2)cc1]

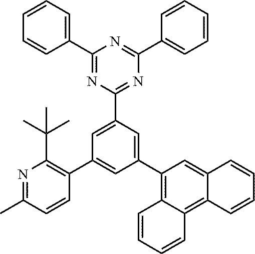 [SMILES: Cc1ccc(-c2cc(-c3nc(-c4ccccc4)nc(-c4ccccc4)n3)cc(-c3cc4ccccc4c4ccccc34)c2)c(C(C)(C)C)n1]

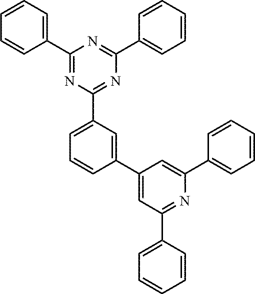 [SMILES: c1ccc(-c2cc(-c3cccc(-c4nc(-c5ccccc5)nc(-c5ccccc5)n4)c3)cc(-c3ccccc3)n2)cc1]

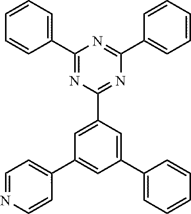 [SMILES: c1ccc(-c2cc(-c3ccncc3)cc(-c3nc(-c4ccccc4)nc(-c4ccccc4)n3)c2)cc1]

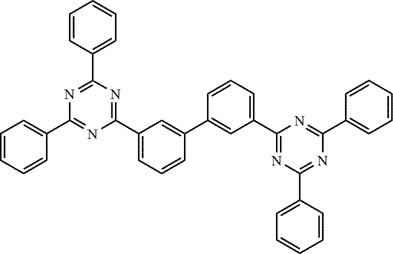 [SMILES: c1ccc(-c2nc(-c3ccccc3)nc(-c3cccc(-c4cccc(-c5nc(-c6ccccc6)nc(-c6ccccc6)n5)c4)c3)n2)cc1]

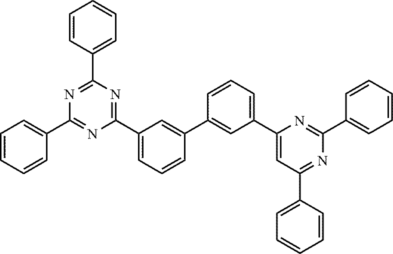 [SMILES: c1ccc(-c2cc(-c3cccc(-c4cccc(-c5nc(-c6ccccc6)nc(-c6ccccc6)n5)c4)c3)nc(-c3ccccc3)n2)cc1]

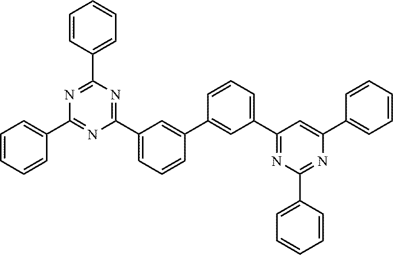 [SMILES: c1ccc(-c2cc(-c3cccc(-c4cccc(-c5nc(-c6ccccc6)nc(-c6ccccc6)n5)c4)c3)nc(-c3ccccc3)n2)cc1]

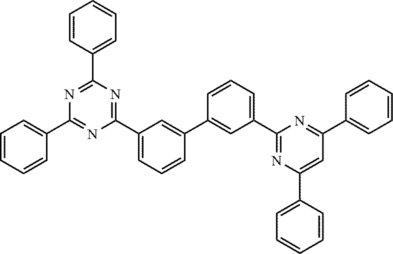 [SMILES: c1ccc(-c2cc(-c3ccccc3)nc(-c3cccc(-c4cccc(-c5nc(-c6ccccc6)nc(-c6ccccc6)n5)c4)c3)n2)cc1]

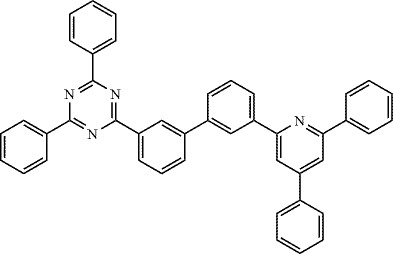 [SMILES: c1ccc(-c2cc(-c3ccccc3)nc(-c3cccc(-c4cccc(-c5nc(-c6ccccc6)nc(-c6ccccc6)n5)c4)c3)c2)cc1]

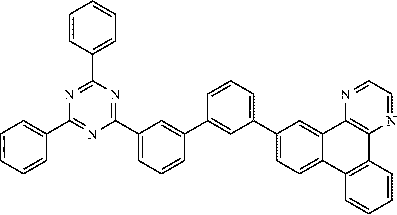 [SMILES: c1ccc(-c2nc(-c3ccccc3)nc(-c3cccc(-c4cccc(-c5ccc6c7ccccc7c7nccnc7c6c5)c4)c3)n2)cc1]

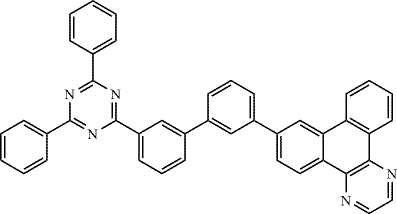 [SMILES: c1ccc(-c2nc(-c3ccccc3)nc(-c3cccc(-c4cccc(-c5ccc6c(c5)c5ccccc5c5nccnc65)c4)c3)n2)cc1]

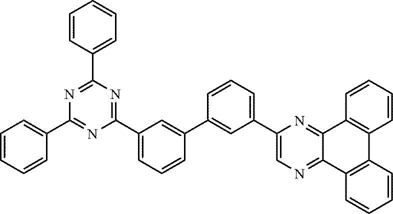 [SMILES: c1ccc(-c2nc(-c3ccccc3)nc(-c3cccc(-c4cccc(-c5cnc6c7ccccc7c7ccccc7c6n5)c4)c3)n2)cc1]

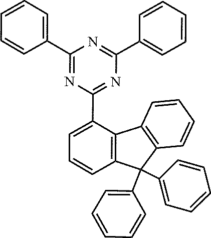 [SMILES: c1ccc(-c2nc(-c3ccccc3)nc(-c3cccc4c3-c3ccccc3C4(c3ccccc3)c3ccccc3)n2)cc1]

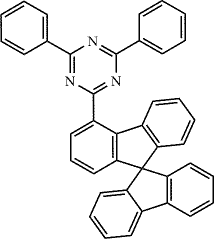 [SMILES: c1ccc(-c2nc(-c3ccccc3)nc(-c3cccc4c3-c3ccccc3C43c4ccccc4-c4ccccc43)n2)cc1]

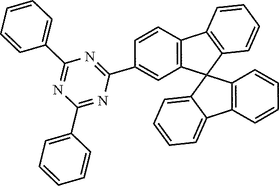 [SMILES: c1ccc(-c2nc(-c3ccccc3)nc(-c3ccc4c(c3)C3(c5ccccc5-c5ccccc53)c3ccccc3-4)n2)cc1]

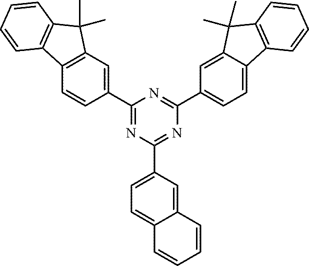 [SMILES: CC1(C)c2ccccc2-c2ccc(-c3nc(-c4ccc5c(c4)C(C)(C)c4ccccc4-5)nc(-c4ccc5ccccc5c4)n3)cc21]

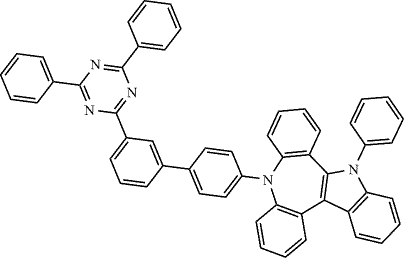 [SMILES: c1ccc(-c2nc(-c3ccccc3)nc(-c3cccc(-c4ccc(N5c6ccccc6-c6c(n(-c7ccccc7)c7ccccc67)-c6ccccc65)cc4)c3)n2)cc1]

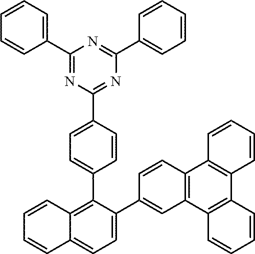 [SMILES: c1ccc(-c2nc(-c3ccccc3)nc(-c3ccc(-c4c(-c5ccc6c7ccccc7c7ccccc7c6c5)ccc5ccccc45)cc3)n2)cc1]

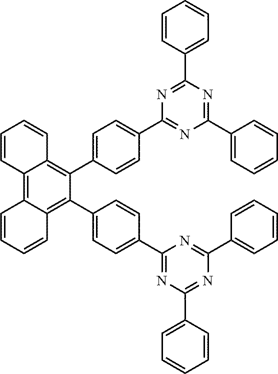 [SMILES: c1ccc(-c2nc(-c3ccccc3)nc(-c3ccc(-c4c(-c5ccc(-c6nc(-c7ccccc7)nc(-c7ccccc7)n6)cc5)c5ccccc5c5ccccc45)cc3)n2)cc1]

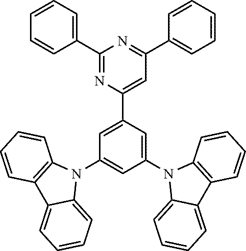 [SMILES: c1ccc(-c2cc(-c3cc(-n4c5ccccc5c5ccccc54)cc(-n4c5ccccc5c5ccccc54)c3)nc(-c3ccccc3)n2)cc1]

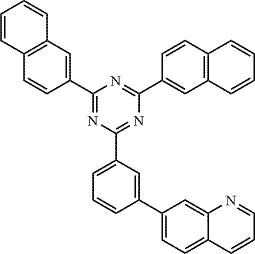 [SMILES: c1cc(-c2ccc3cccnc3c2)cc(-c2nc(-c3ccc4ccccc4c3)nc(-c3ccc4ccccc4c3)n2)c1]

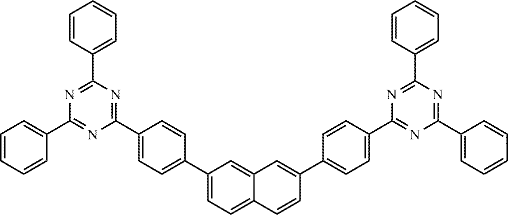 [SMILES: c1ccc(-c2nc(-c3ccccc3)nc(-c3ccc(-c4ccc5ccc(-c6ccc(-c7nc(-c8ccccc8)nc(-c8ccccc8)n7)cc6)cc5c4)cc3)n2)cc1]

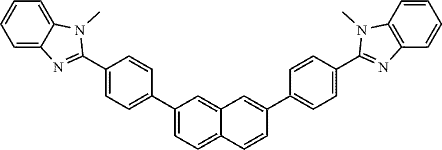 [SMILES: Cn1c(-c2ccc(-c3ccc4ccc(-c5ccc(-c6nc7ccccc7n6C)cc5)cc4c3)cc2)nc2ccccc21]

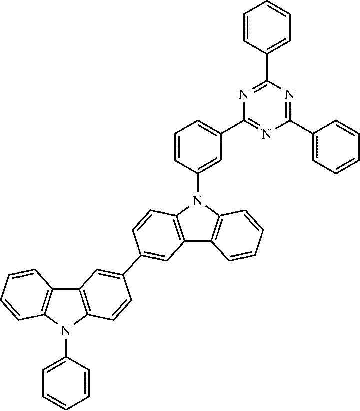 [SMILES: c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1]

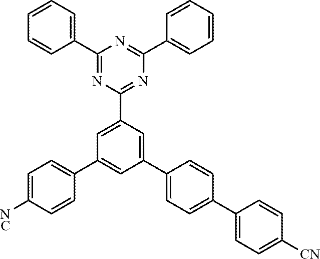 [SMILES: N#Cc1ccc(-c2ccc(-c3cc(-c4ccc(C#N)cc4)cc(-c4nc(-c5ccccc5)nc(-c5ccccc5)n4)c3)cc2)cc1]

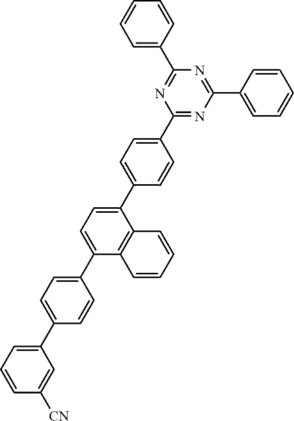 [SMILES: N#Cc1cccc(-c2ccc(-c3ccc(-c4ccc(-c5nc(-c6ccccc6)nc(-c6ccccc6)n5)cc4)c4ccccc34)cc2)c1]

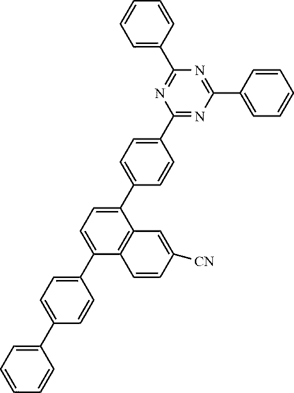 [SMILES: N#Cc1ccc2c(-c3ccc(-c4ccccc4)cc3)ccc(-c3ccc(-c4nc(-c5ccccc5)nc(-c5ccccc5)n4)cc3)c2c1]

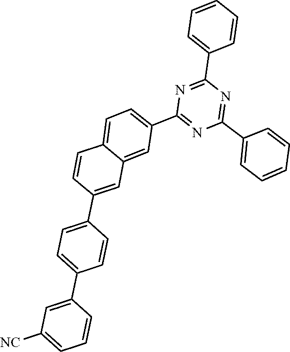

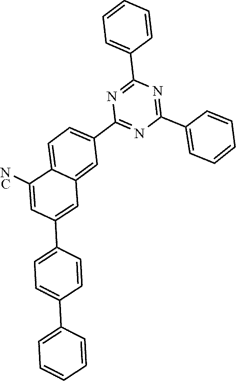

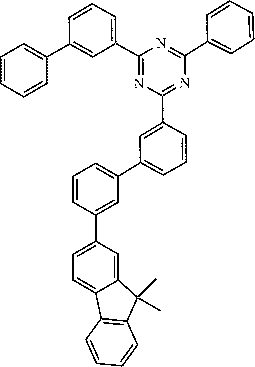 [SMILES: CC1(C)c2ccccc2-c2ccc(-c3cccc(-c4cccc(-c5nc(-c6ccccc6)nc(-c6cccc(-c7ccccc7)c6)n5)c4)c3)cc21]

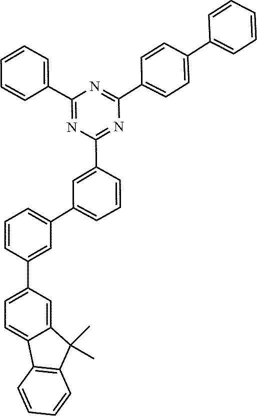 [SMILES: CC1(C)c2ccccc2-c2ccc(-c3cccc(-c4cccc(-c5nc(-c6ccccc6)nc(-c6ccc(-c7ccccc7)cc6)n5)c4)c3)cc21]

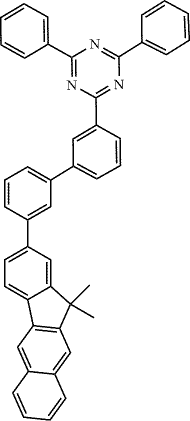 [SMILES: CC1(C)c2cc(-c3cccc(-c4cccc(-c5nc(-c6ccccc6)nc(-c6ccccc6)n5)c4)c3)ccc2-c2cc3ccccc3cc21]

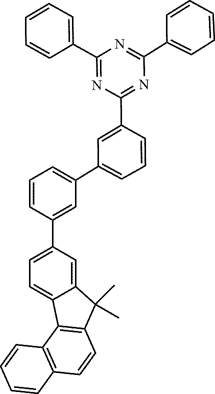 [SMILES: CC1(C)c2cc(-c3cccc(-c4cccc(-c5nc(-c6ccccc6)nc(-c6ccccc6)n5)c4)c3)ccc2-c2c1ccc1ccccc21]

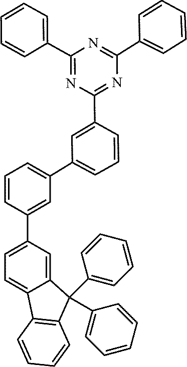 [SMILES: c1ccc(-c2nc(-c3ccccc3)nc(-c3cccc(-c4cccc(-c5ccc6c(c5)C(c5ccccc5)(c5ccccc5)c5ccccc5-6)c4)c3)n2)cc1]

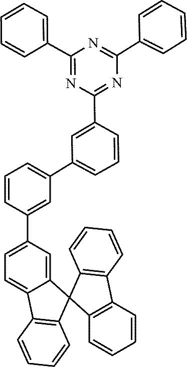 [SMILES: c1ccc(-c2nc(-c3ccccc3)nc(-c3cccc(-c4cccc(-c5ccc6c(c5)C5(c7ccccc7-c7ccccc75)c5ccccc5-6)c4)c3)n2)cc1]

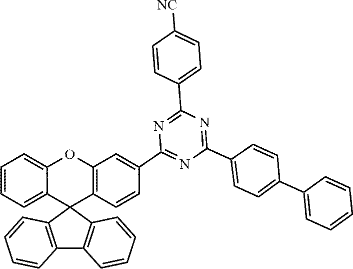

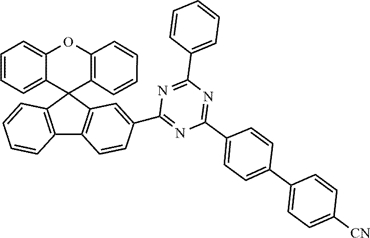 [SMILES: N#Cc1ccc(-c2ccc(-c3nc(-c4ccccc4)nc(-c4ccc5c(c4)C4(c6ccccc6Oc6ccccc64)c4ccccc4-5)n3)cc2)cc1]

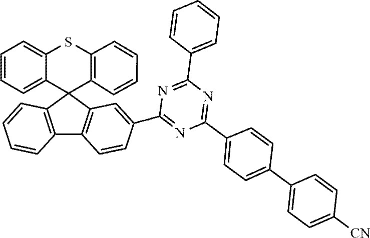 [SMILES: N#Cc1ccc(-c2ccc(-c3nc(-c4ccccc4)nc(-c4ccc5c(c4)C4(c6ccccc6Sc6ccccc64)c4ccccc4-5)n3)cc2)cc1]

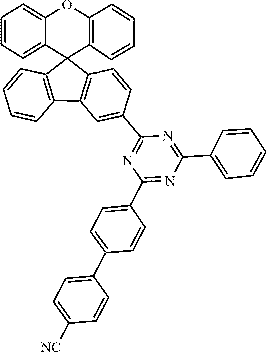

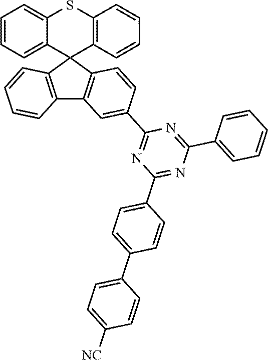

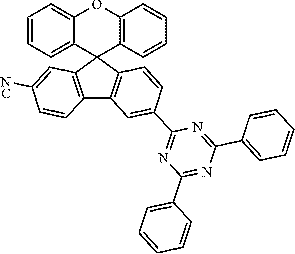

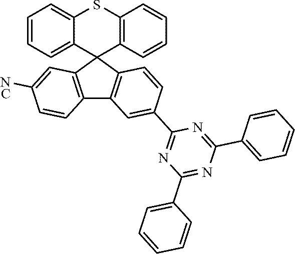

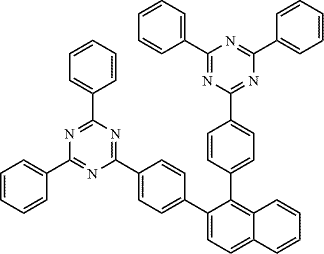 [SMILES: c1ccc(-c2nc(-c3ccccc3)nc(-c3ccc(-c4ccc5ccccc5c4-c4ccc(-c5nc(-c6ccccc6)nc(-c6ccccc6)n5)cc4)cc3)n2)cc1]

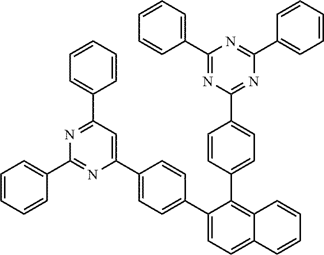 [SMILES: c1ccc(-c2cc(-c3ccc(-c4ccc5ccccc5c4-c4ccc(-c5nc(-c6ccccc6)nc(-c6ccccc6)n5)cc4)cc3)nc(-c3ccccc3)n2)cc1]

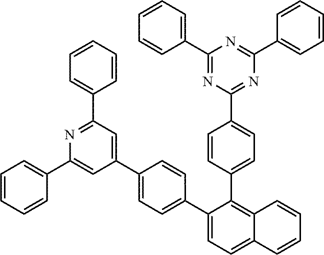 [SMILES: c1ccc(-c2cc(-c3ccc(-c4ccc5ccccc5c4-c4ccc(-c5nc(-c6ccccc6)nc(-c6ccccc6)n5)cc4)cc3)cc(-c3ccccc3)n2)cc1]

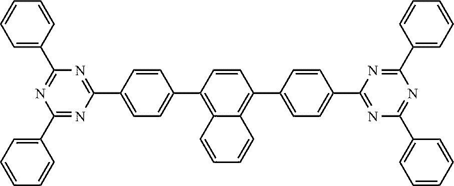 [SMILES: c1ccc(-c2nc(-c3ccccc3)nc(-c3ccc(-c4ccc(-c5ccc(-c6nc(-c7ccccc7)nc(-c7ccccc7)n6)cc5)c5ccccc45)cc3)n2)cc1]

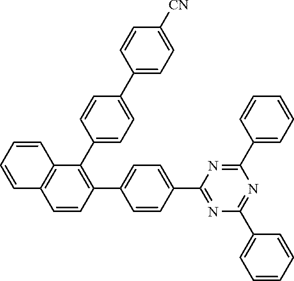 [SMILES: N#Cc1ccc(-c2ccc(-c3c(-c4ccc(-c5nc(-c6ccccc6)nc(-c6ccccc6)n5)cc4)ccc4ccccc34)cc2)cc1]

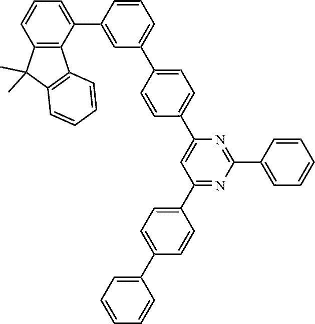 [SMILES: CC1(C)c2ccccc2-c2c(-c3cccc(-c4ccc(-c5cc(-c6ccc(-c7ccccc7)cc6)nc(-c6ccccc6)n5)cc4)c3)cccc21]

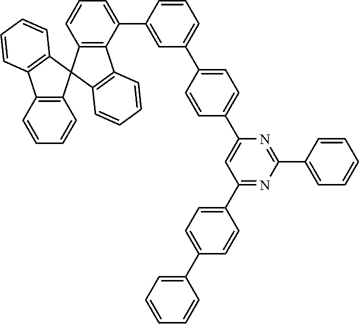 [SMILES: c1ccc(-c2ccc(-c3cc(-c4ccc(-c5cccc(-c6cccc7c6-c6ccccc6C76c7ccccc7-c7ccccc76)c5)cc4)nc(-c4ccccc4)n3)cc2)cc1]

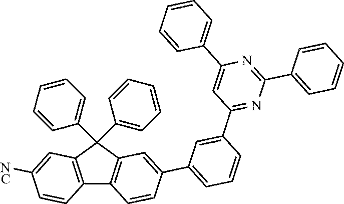

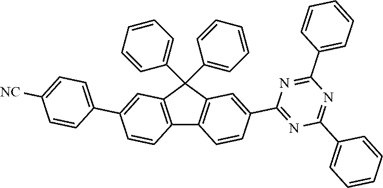

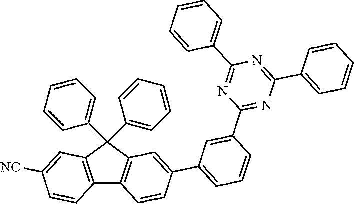

An example embodiment may provide an organic light emitting diode including a light emitting layer as an organic layer.

Another example embodiment may provide an organic light emitting diode including a light emitting layer and a hole transport region as an organic layer.

Another example embodiment may provide an organic light emitting diode including a light emitting layer and an electron transport region as an organic layer.

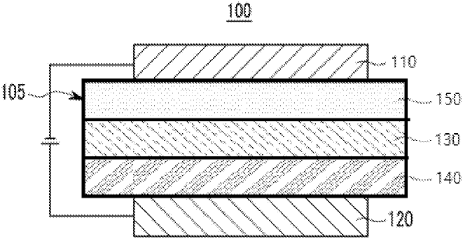

The organic light emitting diode according to an example embodiment may include a hole transport region 140 and an electron transport region 150 in addition to the light emitting layer 130 as the organic layer 105, as shown in FIG. 1.

The organic light emitting diode according to an example embodiment may further include an electron injection layer (not shown), a hole injection layer (not shown), etc., in addition to the light emitting layer as the aforementioned organic layer.

The organic light emitting diode 100 may be produced by forming an anode or a cathode on a substrate, forming an organic layer using a dry film formation method such as a vacuum deposition method (evaporation), sputtering, plasma plating, and ion plating, and forming a cathode or an anode thereon.

The organic light emitting diode may be applied to an organic light emitting display device.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

Hereinafter, starting materials and reactants used in the Examples and Synthesis Examples were purchased from Sigma-Aldrich Co. Ltd., TCI Inc., or Tokyo Chemical Industry, or were synthesized by general methods, unless otherwise stated.

### Preparation of Compound for Organic Optoelectronic Device

### Synthesis Example 1: Synthesis of Compound 1-1

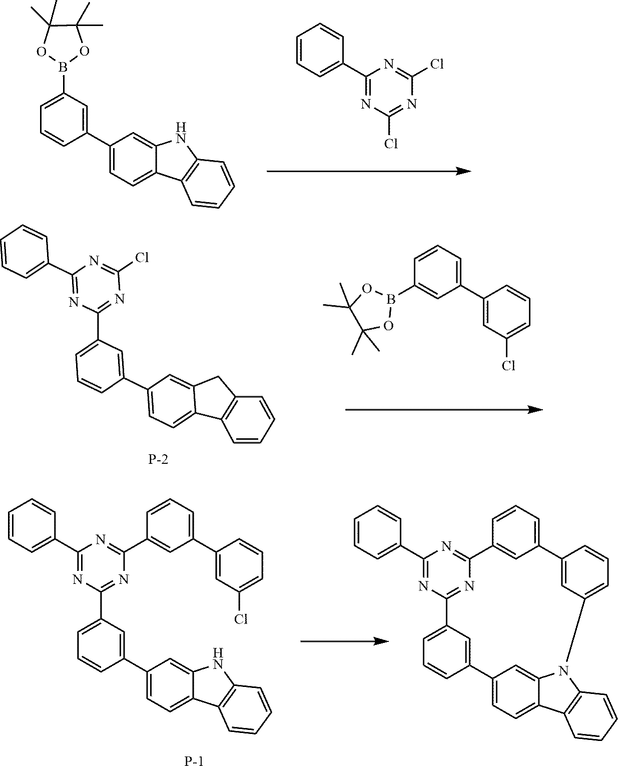

### 1st Step: Synthesis of Intermediate P-2

2-[3-(4,4,5,5-tetramethyl-1,3,2-dioxaborolan-2-yl)phenyl]-9H-carbazole (50 g), 2,4-dichloro-6-phenyl-1,3,5-triazine (31 g), Pd(PPh₃)₄ (7.8 g), and K₂CO₃ (56 g) were injected into a flask together with THF (500 mL) and distilled water (165 mL) and refluxed at 80° C. After 12 hours, the reaction was terminated and then the resultant was diluted with DCM (dichloromethane), washed 3 times with brine, and dried with MgSO₄. 35 g of Intermediate P-2 was obtained using column chromatography.

### 2nd Step: Synthesis of Intermediate P-1

Intermediate P-2 (35 g), 2-(3′-chloro[1,1′-biphenyl]-3-yl)-4,4,5,5-tetramethyl-1,3,2-dioxaborolane (26 g), Pd(PPh₃)₄ (4.7 g), and K₂CO₃ (34 g) were introduced into a flask together with THF (300 mL) and distilled water (100 mL) and refluxed at 80° C. After 12 hours, the reaction was terminated and then the resultant was diluted with DCM, washed 3 times with brine, and dried with MgSO₄. 33 g of Intermediate P-1 was obtained by column chromatography.

### 3rd Step: Synthesis of Compound 1-1

Intermediate P-1 (33 g), Pd(OAc)₂ (1.3 g), P(t-Bu)₃ (2.3 g), and K₂CO₃ (24 g) were injected into a flask together with xylene (500 mL) and refluxed at 150° C.

After 12 hours, the reaction was terminated and then the resultant was diluted with DCM, washed 3 times with brine, and dried with MgSO₄. 17 g of compound 1-1 was obtained by column chromatography.

Synthesis Example 2: Synthesis of Compound 1-17

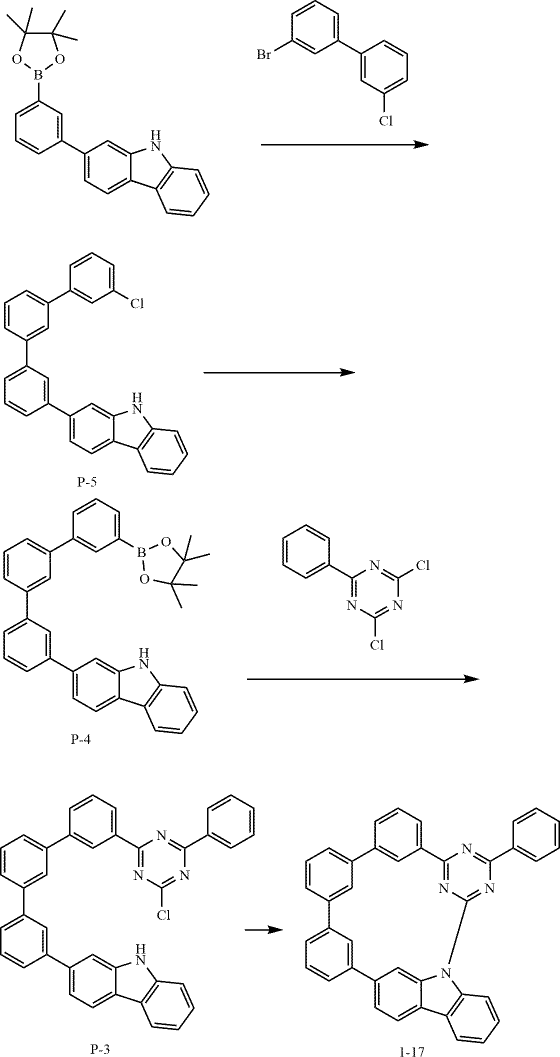

### 1st Step: Synthesis of Intermediate P-5

00163] 2-[3-(4,4,5,5-tetramethyl-1,3,2-dioxaborolan-2-yl)phenyl]-9H-carbazole (50 g), 3-bromo-3′-chloro-1, 1′-biphenyl (36 g), Pd(PPh₃)₄ (7.8 g), and K₂CO₃ (56 g) were injected into a flask together with THF (500 mL) and distilled water (165 mL) and refluxed at 80° C. After 12 hours, the reaction was terminated and then the resultant was diluted with DCM, washed 3 times with brine, and dried with MgSO₄. 29 g of Intermediate P-5 was obtained using column chromatography.

### 2nd Step: Synthesis of Intermediate P-4

Intermediate P-5 (29 g), bis(pinacolato)diboron 34 g, Pd(OAc)₂ (1.5 g), K₃PO₄ (46 g),and s-Phos (2.8 g) were injected into a flask together with 1,4-dioxane (300 mL) and refluxed at 100° C. After 12 hours, the reaction was terminated and then the resultant was diluted with DCM, washed 3 times with brine, and dried with MgSO₄. 28 g of Intermediate P-4 was obtained using column chromatography.

### 3rd Step: Synthesis of Intermediate P-3

Intermediate P-4 (28 g), 2,4-dichloro-6-phenyl-1,3,5-triazine (12 g), Pd(PPh₃)₄ (3.2 g),and K₂CO₃ (23 g) were injected into a flask together with THF (300 mL) and distilled water (100 mL) and refluxed at 80° C. After 12 hours, the reaction was terminated and then the resultant was diluted with DCM, washed 3 times with brine, and dried with MgSO₄. 19 g of Intermediate P-3 was obtained using column chromatography.

### 4th Step: Synthesis of Compound 1-17

Intermediate P-3 (19 g), Pd(OAc)₂ (0.8 g), P(t-Bu)₃ (1.3 g), and K₂CO₃ (13 g) were injected into a flask together with xylene (200 mL) and refluxed at 150° C. After 12 hours, the reaction was terminated and then the resultant was diluted with DCM, washed 3 times with brine, and dried with MgSO₄. 11 g of Compound 1-17 was obtained using column chromatography.

Synthesis Example 3: Synthesis of Compound 1-33

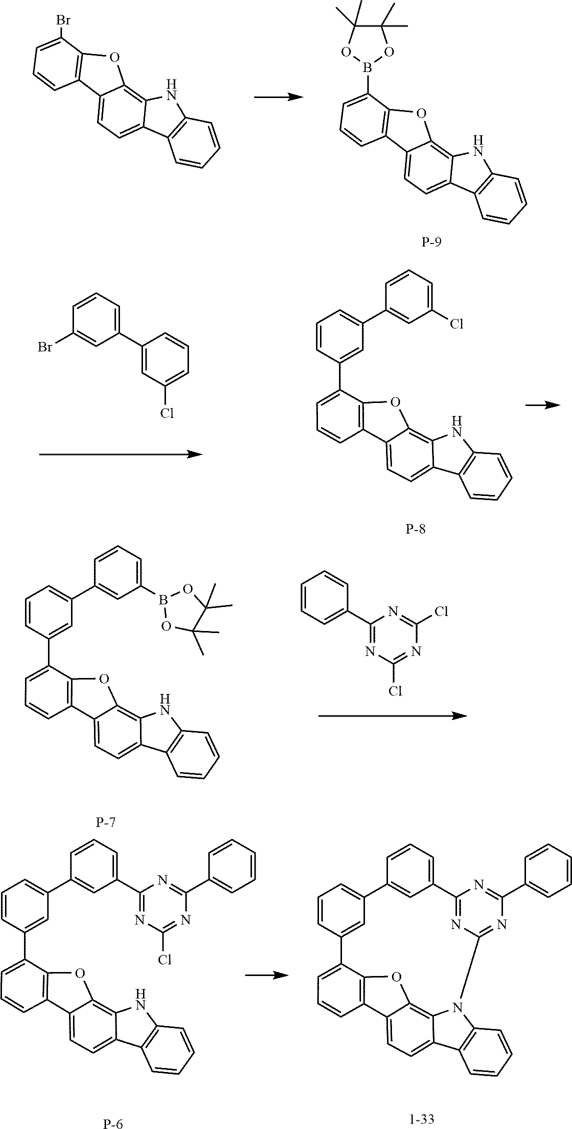

### 1st Step: Synthesis of Intermediate P-9

10-bromo-12H-benzofuro[2,3-a]carbazole (50 g), bis(pinacolato)diboron (75 g), Pd(OAc)₂ (3.3 g), K₃PO₄ (101 g), and s-Phos (6.2 g) were injected into a flask together with 1,4-dioxane (700 mL) and refluxed at 100° C. After 12 hours, the reaction was terminated and then the resultant was diluted with DCM, washed 3 times with brine, and dried with MgSO₄. 48 g of Intermediate P-9 was obtained using column chromatography.

### 2nd Step: Synthesis of Intermediate P-8

Intermediate P-9 (48 g), 3-bromo-3′-chloro-1,1′-biphenyl (34 g), Pd(PPh₃)₄ (7.3 g), and K₂CO₃ (53 g) were injected into a flask together with THF (500 mL) and distilled water (165 mL) and refluxed at 80° C. After 12 hours, the reaction was terminated and then the resultant was diluted with DCM, washed 3 times with brine, and dried with MgSO₄. 39 g of Intermediate P-8 was obtained using column chromatography.

### 3rd Step: Synthesis of Intermediate P-7

Intermediate P-8 (39 g), bis(pinacolato)diboron (45 g), Pd(OAc)₂ (2.0 g), K₃PO₄ (61 g),and s-Phos (3.7 g) were injected into a flask together with 1,4-dioxane (400 mL) and refluxed at 100° C. After 12 hours, the reaction was terminated and then the resultant was diluted with DCM, washed 3 times with brine, and dried with MgSO₄. 44 g of Intermediate P-7 was obtained using column chromatography.

### 4th Step: Synthesis of Intermediate P-6

Intermediate P-7 (44 g), 2,4-dichloro-6-phenyl-1,3,5-triazine (19 g), Pd(PPh₃)₄ (5.1 g),and K₂CO₃ (36.3 g) were injected into a flask together with THF (300 mL) and distilled water (100 mL) and refluxed at 80° C. After 12 hours, the reaction was terminated and then the resultant was diluted with DCM, washed 3 times with brine, and dried with MgSO₄. 30 g of Intermediate P-6 was obtained using column chromatography.

### 5th Step: Synthesis of Compound 1-33

Intermediate P-6 (30 g), Pd(OAc)₂ (1.1 g), P(t-Bu)₃ (1.8 g), and K₂CO₃ (18 g) were injected into a flask together with xylene (300 mL) and refluxed at 150° C. After 12 hours, the reaction was terminated and then the resultant was diluted with DCM, washed 3 times with brine, and dried with MgSO₄. 22 g of Compound 1-33 was obtained using column chromatography.

Comparative Synthesis Example 1: Synthesis of Compound C1

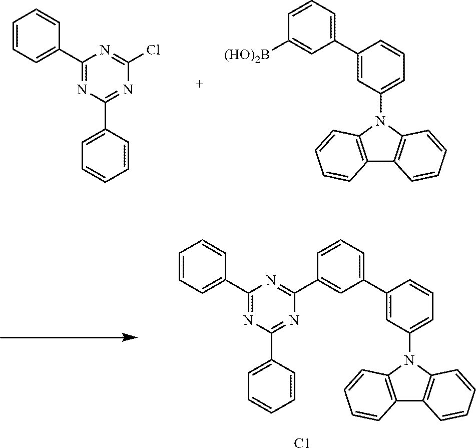

2-Chloro-4,6-diphenyl-1,3,5-triazine (50 g), [3′-(9H-carbazol-9-yl)[1,1′-biphenyl]-3-yl]-boronic acid, (68 g), Pd(PPh₃)₄ (11 g), and K₂CO₃ (77 g) were injected into a flask together with THF (450 mL) and distilled water (150 mL) and refluxed at 80° C. After 12 hours, the reaction was terminated and then the resultant was diluted with DCM, washed 3 times with brine, and dried with MgSO₄. 82 g of Compound C1 was obtained using column chromatography.

Comparative Synthesis Example 2: Synthesis of Compound C2

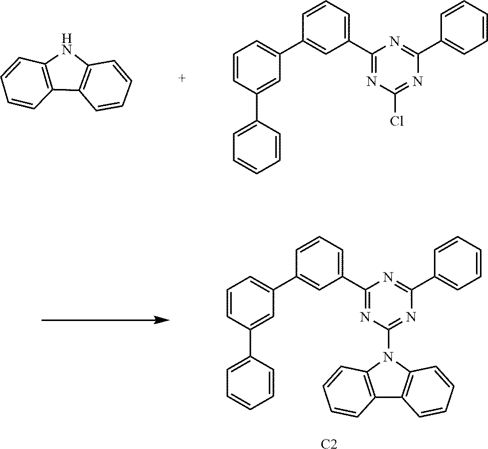

Carbazole (20 g), 2-chloro-4-phenyl-6-[1,1′:3′,1″-terphenyl]-3-yl-1,3,5-triazine (50 g), Pd(OAc)₂ (2.7 g), P(t-Bu)₃ (4.8 g), and K₂CO₃ (50 g) were injected into a flask together with xylene (200 mL) and refluxed at 150° C. After 12 hours, the reaction was terminated and then the resultant was diluted with DCM, washed 3 times with brine, and dried with MgSO₄. 52 g of Compound C2 was obtained using column chromatography.

Comparative Synthesis Example 3: Synthesis of Compound C3

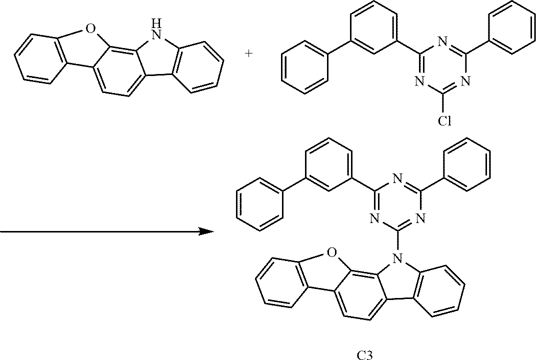

12H-Benzofuro[2,3-a]carbazole (20 g), 2-([1,1′-biphenyl]-3-yl)-4-chloro-6-phenyl-1,3,5-triazine (27 g), Pd(OAc)₂ (1.5 g), P(t-Bu)₃ (2.6 g), and K₂CO₃ (27 g) were injected into a flask together with xylene (100 mL) and refluxed at 150° C. After 12 hours, the reaction was terminated and then the resultant was diluted with DCM, washed 3 times with brine, and dried with MgSO₄. 39 g of Compound C3 was obtained using column chromatography.

### Manufacture of Organic Light Emitting Diode

### Example 1

Referring to the list of compounds below, a glass substrate coated with ITO (indium tin oxide) was washed with distilled water and ultrasonic waves. After washing with the distilled water, the glass substrate was washed with a solvent such as isopropyl alcohol, acetone, methanol, and the like ultrasonically and dried and then, moved to a plasma cleaner, cleaned by using oxygen plasma for 10 minutes, and moved to a vacuum depositor. The thus prepared ITO transparent electrode was used as an anode, Compound A doped with 3% NDP-9 (Novaled GmbH) was vacuum-deposited on the ITO substrate to form a 100 Å-thick hole injection layer, and Compound A was deposited on the hole injection layer to a thickness of 1350 Å to form a hole transport layer. Compound B was deposited on the hole transport layer to a thickness of 350 Å to form a hole transport auxiliary layer. On the hole transport auxiliary layer, Compound 1-1 of Synthesis Example 1 was used as a host and 7 wt% of PhGD was used as a dopant to form a 400 Å-thick light emitting layer by vacuum deposition. Subsequently, Compound C was deposited to form a 50 Å-thick electron transport auxiliary layer on the light emitting layer, and Compound D and LiQ were simultaneously vacuum-deposited in a weight ratio of 1:1 to form a 300 Å-thick electron transport layer. On the electron transport layer, LiQ and Al were sequentially vacuum-deposited to be 15 Å-thick and 1200 Å-thick, manufacturing an organic light emitting diode.

The structure was ITO / Compound A (3% NDP-9 doping, 100 Å)/ Compound A (1350 Å)/ Compound B (350 Å)/ EML [Compound 1-1:PhGD=93:7 wt%)] (400 Å)/Compound C (50 Å)/Compound D:LiQ (300 Å)/LiQ (15 Å) / Al (1200 Å).

Compound A: N-(biphenyl-4-yl)-9,9-dimethyl-N-(4-(9-phenyl-9H-carbazol-3-yl)phenyl)-9H-fluoren-2-amine

Compound B: N-[4-(4-Dibenzofuranyl)phenyl]-N-[4-(9-phenyl-9H-fluoren-9-yl)phenyl][1,1′-biphenyl]-4-amine

Compound C: 2,4-Diphenyl-6-(4′,5′,6′-triphenyl[1,1′:2′,1″:3″,1′″:3″′,1⁗-quinquephenyl]-3⁗-yl)-1,3,5-triazine

Compound D: 2-(1,1′-Biphenyl-4-yl)-4-(9,9-diphenylfluoren-4-yl)-6-phenyl-1,3,5-triazine

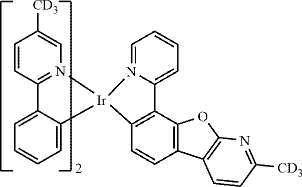

### Example 2

An organic light emitting diode was manufactured in the same manner as Example 1 except that Compound 1-17 was used instead of Compound 1-1.

### Example 3

An organic light emitting diode was manufactured in the same manner as Example 1 except that Compound 1-33 was used instead of Compound 1-1.

### Example 4

Referring to the compounds below, a glass substrate coated with ITO (indium tin oxide) was washed with distilled water and ultrasonic waves. After washing with the distilled water, the glass substrate was washed with a solvent such as isopropyl alcohol, acetone, methanol, and the like ultrasonically and dried and then, moved to a plasma cleaner, cleaned by using oxygen plasma for 10 minutes, and moved to a vacuum depositor. The thus prepared ITO transparent electrode was used as an anode, Compound A doped with 3% NDP-9 (Novaled GmbH) was vacuum-deposited on the ITO substrate to form a 100 Å-thick hole injection layer, and Compound A was deposited on the hole injection layer to a thickness of 1350 Å to form a hole transport layer. Compound E was deposited to be a thickness of 350 Å on the hole transport layer to form a hole transport auxiliary layer. On the hole transport auxiliary layer, a mixture of Compound 1-1 of Synthesis Example 1 and Compound A-136 in a weight ratio of 3:7 was used as a host, and was doped with 10 wt% of PhGD as a dopant to form a 400 Å-thick light emitting layer by vacuum deposition. Subsequently, Compound F was deposited to form a 50 Å-thick electron transport auxiliary layer on the light emitting layer, and Compound G and LiQ were simultaneously vacuum-deposited in a weight ratio of 1:1 to form a 300 Å-thick electron transport layer. On the electron transport layer, LiQ and Al were sequentially vacuum-deposited to be 15 Å-thick and 1200 Å-thick, manufacturing an organic light emitting diode.

The structure was ITO / Compound A (3% NDP-9 doping, 100 Å) / Compound A (1350 Å)/Compound E (350 Å) /EML [[{Host (Compound 1-1: Compound A-136)} : {Dopant (PhGD)}=90:10 wt%] (400 Å)/Compound F (50 Å)/Compound G:LiQ (300 Å) / LiQ (15 Å) / Al (1200 Å).

Compound E: N,N-bis(9,9-dimethyl-9H-fluoren-4-yl)-9,9-spirobi(fluorene)-2-amine

Compound F: 2-[3′-(9,9-Dimethyl-9H-fluoren-2-yl)[1,1′-biphenyl]-3-yl]-4,6-diphenyl-1,3,5-triazine

Compound G: 2-[4-[4-(4′-cyano-1,1′-biphenyl-4-yl)-1-naphthyl]phenyl]-4,6-diphenyl-1,3,5-triazine

### Example 5

An organic light emitting diode was manufactured in the same manner as in Example 4, except that Compound 1-17 and Compound A-136 were used in a weight ratio of 3:7 instead of Compound 1-1.

### Example 6

An organic light emitting diode was manufactured in the same manner as in Example 4, except that Compound 1-33 and Compound A-136 were used in a weight ratio of 3:7 instead of Compound 1-1.

### Comparative Example 1

An organic light emitting diode was manufactured in the same manner as in Example 1, except that Compound C1 was used instead of Compound 1-1.

### Comparative Example 2

An organic light emitting diode was manufactured in the same manner as in Example 1, except that Compound C2 was used instead of Compound 1-1.

### Comparative Example 3

An organic light emitting diode was manufactured in the same manner as in Example 1, except that Compound C3 was used instead of Compound 1-1.

### Comparative Example 4

An organic light emitting diode was manufactured in the same manner as in Example 4, except that Compound C1 and Compound A-136 were used in a weight ratio of 3:7 instead of Compound 1-1.

### Comparative Example 5

An organic light emitting diode was manufactured in the same manner as in Example 4, except that Compound C2 and Compound A-136 were used in a weight ratio of 3:7 instead of Compound 1-1.

### Comparative Example 6

An organic light emitting diode was manufactured in the same manner as in Example 4, except that Compound C3 and Compound A-136 were used in a weight ratio of 3:7 instead of Compound 1-1.

### Evaluation

The driving voltage and life-span characteristics of the organic light emitting diodes according to Examples 1 to 6 and Comparative Examples 1 to 6 were evaluated.

Specific measurement methods are as follows, and the results are shown in Table 1.

### Measurement of Current Density Change Depending on Voltage Change

The obtained organic light emitting diodes were measured regarding a current value flowing in the unit device, while increasing the voltage from 0 V to 10 V using a current-voltage meter (Keithley 2400). The measured current value is divided by area to provide the results.

### Measurement of Luminance Change Depending on Voltage Change

Luminance was measured by using a luminance meter (Minolta Cs-1000A), while the voltage of the organic light emitting diodes was increased from 0 V to 10 V.

### Measurement of Luminous Efficiency

Luminous Efficiency (cd/A) at the same current density (10 mA/cm²) is calculated by using the luminance and current density from the items (1) and (2) and voltages.

### Measurement of Life-span

The result was obtained by measuring the time for the luminance (cd/m²) to be maintained at 18000 cd/m² and the current efficiency (cd/A) to decrease to 97%.

### Measurement of Driving Voltage

A driving voltage of each diode was measured by using a current-voltage meter (Keithley 2400) at 15 mA/cm².

TABLE 1

| Nos. | Compound | Driving voltage (%) | Luminous efficiency (%) | Life-span (%) |
|---|---|---|---|---|
| Example 1 | 1-1 | 90.8 | 109.3 | 125.0 |
| Example 2 | 1-17 | 98.0 | 103.7 | 115.0 |
| Example 3 | 1-33 | 89.8 | 109.3 | 130.0 |
| Comparative Example 1 | C1 | 100.0 | 100.0 | 100.0 |
| Comparative Example 2 | C2 | 105.5 | 98.1 | 90.0 |
| Comparative Example 3 | C3 | 101.0 | 98.1 | 105.0 |

TABLE 2

| Nos. | Compound (first host) | Compound (second host) | Driving voltage (%) | Luminous efficiency (%) | Life-span (%) |
|---|---|---|---|---|---|
| Example 4 | 1-1 | A-136 | 93.9 | 111.2 | 140.0 |
| Example 5 | 1-17 | A-136 | 98.7 | 112.4 | 133.0 |
| Example 6 | 1-33 | A-136 | 94.7 | 111.8 | 137.0 |
| Comparative Example 4 | C1 | A-136 | 100.0 | 100.0 | 100.0 |
| Comparative Example 5 | C2 | A-136 | 104.0 | 101.6 | 92.0 |
| Comparative Example 6 | C3 | A-136 | 100.8 | 100.2 | 102.0 |

Referring to Tables 1 and 2, the compounds according to the Examples, which included a macro compound rather than an open structure, had improved driving, efficiency, and life-span. Without being bound by theory, it is believed that this increases mobility of holes and electrons injected into the EML layer due to the extended resonance, and stabilizes it through delocalization, thereby increasing the life-span. In addition, as the molecule is cyclized, movement of the molecule is limited, which inevitably results in small structural changes in the excited state and the ground state. Due to this, the side reaction pathway in the excited state is limited, and thus it shows high efficiency and long life-span.

As described above, an example embodiment may provide a compound for an organic optoelectronic device capable of implementing an organic optoelectronic device having high efficiency and a long life-span.

An example embodiment provides a composition for an organic optoelectronic device including the compound for the organic optoelectronic device. Another example embodiment provides an organic optoelectronic device including the compound for the organic optoelectronic device or the composition for the organic optoelectronic device. Another example embodiment provides a display device including the organic optoelectronic device.

According to an example embodiment, an organic optoelectronic device having high efficiency and long life-span may be realized.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

### Description of Symbols

100: organic light emitting diode

105: organic layer

110: cathode

120: anode

130: light emitting layer

140: hole transport region

150: electron transport region

What is claimed is:

1. A compound for an organic optoelectronic device, the compound being represented by Chemical Formula 1: 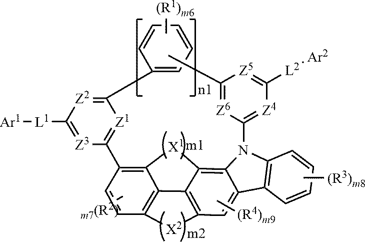
wherein, in Chemical Formula 1,
m1 and m2 are each independently an integer of 0 or 1,
X¹ and X² are each independently NR^{g}, O, S, CR^{a}R^{b}, or SiR^{c}R^{d},
n1 is an integer of 1 to 5,
Z¹ to Z⁶ are each independently N or CR^{e},
at least two of Z¹ to Z³ are N; at least two of Z⁴ to Z⁶ are N; or at least two of Z¹ to Z³ are N and at least two of Z⁴ to Z⁶ are N,
R^{a}, R^{b}, R^{c}, R^{d} and R^{g} are each independently a substituted or unsubstituted C1 to C30 alkyl group, or a substituted or unsubstituted C6 to C30 aryl group,
R^{e} and R¹ toR⁴ are each independently hydrogen, deuterium, a halogen, a substituted or unsubstituted C1 to C30 alkyl group, or a substituted or unsubstituted C6 to C30 aryl group,
m6 and m8 are each independently an integer of 1 to 4,
m7 is an integer of 1 to 4,
m9 is an integer of 1 to 3,
Ar¹ and Ar² are each independently a substituted or unsubstituted C6 to C30 aryl group or a substituted or unsubstituted C2 to C30 heterocyclic group, and
L¹ and L² are each independently a single bond, a substituted or unsubstituted C6 to C30 arylene group, or a substituted or unsubstituted C2 to C30 heterocyclic group.

2. The compound as claimed in claim 1, wherein Chemical Formula 1 is represented by any one of Chemical Formula 1A to Chemical Formula 1D: 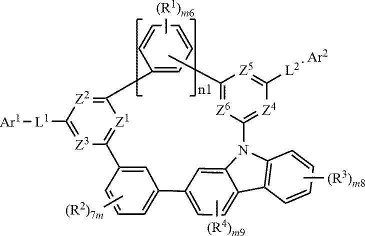 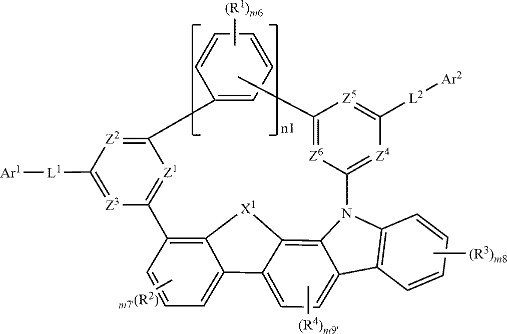 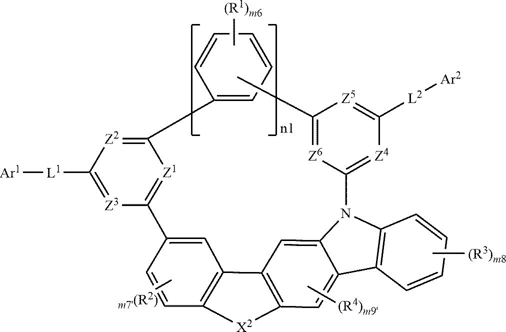 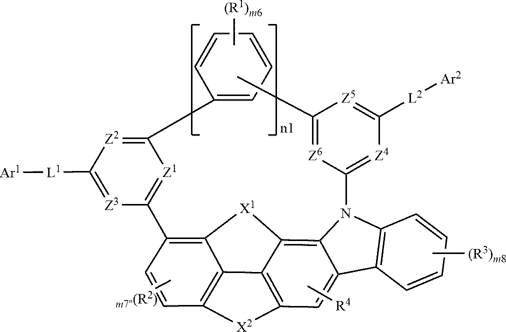
wherein, in Chemical Formula 1A to Chemical Formula 1D,
Ar¹, Ar², L¹, L², X¹, X², Z¹ to Z⁶, n1, m6 to m9, and R¹ to R⁴ are as defined for Chemical Formula 1,
m7′ is an integer of 1 to 3,
m7″ is an integer of 1 or 2, and
m9′ is an integer of 1 or 2.

3. The compound as claimed in claim 1, wherein Chemical Formula 1 is represented by any one of Chemical Formula 1-I, Chemical Formula 1-II, and Chemical Formula 1-III: 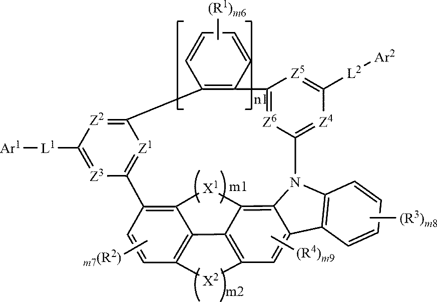 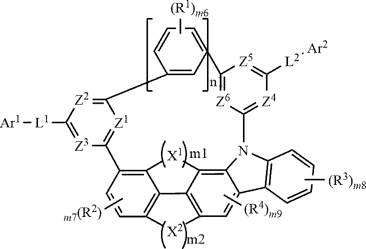 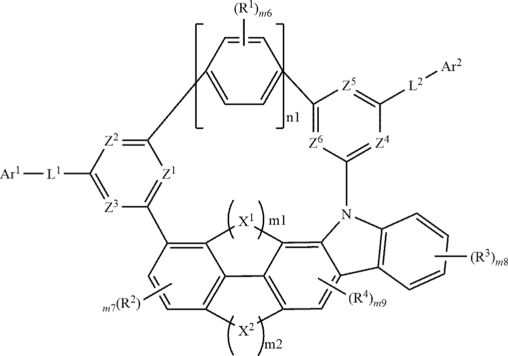
wherein, in Chemical Formula 1-I, Chemical Formula 1-II, and Chemical Formula 1-III,
Ar¹, Ar², L¹, L², X¹, X², m1, m2, m6 to m9, Z¹ to Z⁶, n1, and R¹ toR⁴ are as defined for Chemical Formula 1.

4. The compound as claimed in claim 1, wherein n1 is an integer of 1 to 3.

5. The compound as claimed in claim 4, wherein Chemical Formula 1 is represented by any one of Chemical Formula 1-IIa, Chemical Formula 1-IIb, and Chemical Formula 1-IIc: 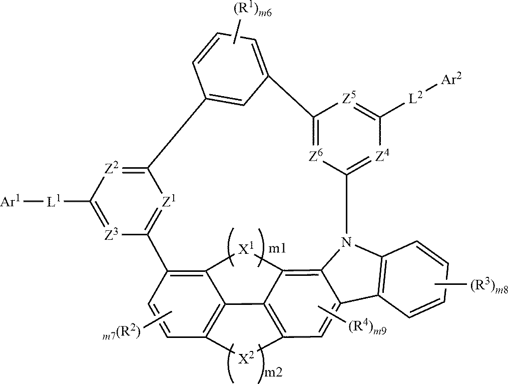 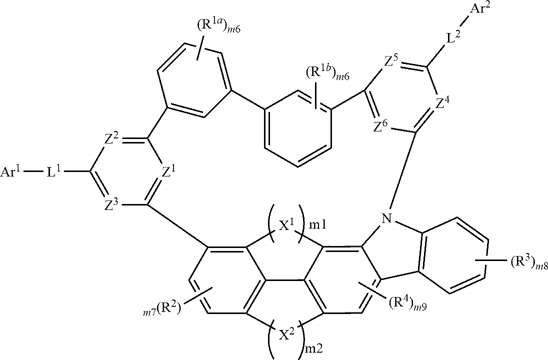 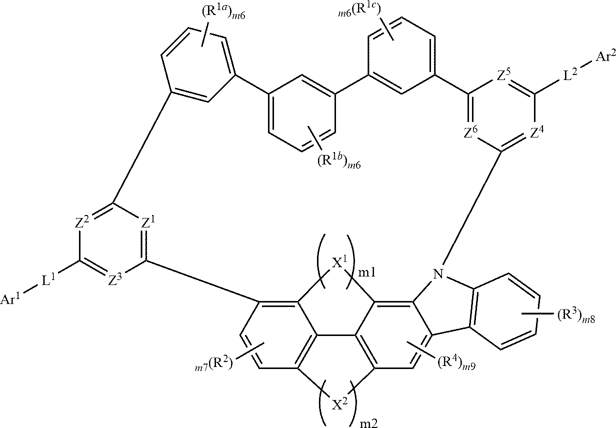
wherein, in Chemical Formula 1-IIa, Chemical Formula 1-IIb, and Chemical Formula 1-IIc,
Ar¹, Ar², L¹, L², X¹, X², m1, m2, m6 to m9, Z¹ to Z⁶, and R¹ to R⁴ are as defined for Chemical Formula 1, and
R^{1a}, R^{1b}, and R^{1c} have the same definitions as R¹.

6. The compound as claimed in claim 1, wherein X¹ and X² are each independently O or S.

7. The compound as claimed in claim 1, wherein Ar¹ and Ar² are each independently a substituted or unsubstituted phenyl group, a substituted or unsubstituted biphenyl group, a substituted or unsubstituted terphenyl group, a substituted or unsubstituted naphthyl group, a substituted or unsubstituted fluorenyl group, a substituted or unsubstituted triphenylene group, a substituted or unsubstituted dibenzofuranyl group, a substituted or unsubstituted dibenzothiophenyl group, or a substituted or unsubstituted dibenzosilolyl group.

8. The compound as claimed in claim 1, wherein ^{∗}-L¹-Ar¹ and ^{∗}-L²-Ar² are each independently selected from the groups of Group I: 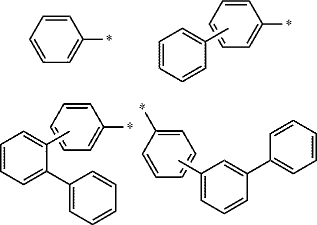 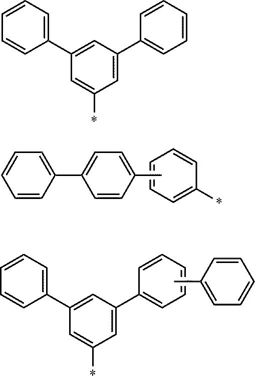 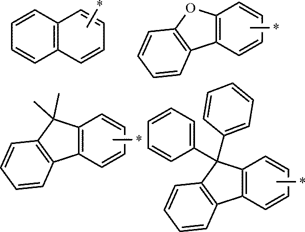 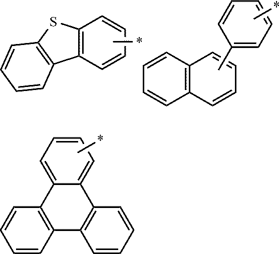
wherein, in Group I, * is a linking point.

9. The compound as claimed in claim 1, wherein the compound is one of compounds of Group 1: 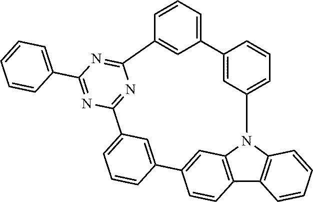 [SMILES: c1ccc(-c2nc3nc(n2)-c2cccc(c2)-c2ccc4c5ccccc5n(c4c2)-c2cccc(c2)-c2cccc-3c2)cc1] 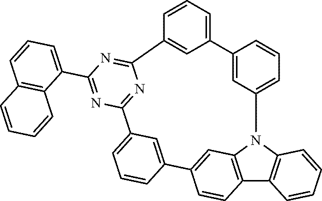 [SMILES: c1cc2cc(c1)-c1nc(nc(-c3cccc4ccccc34)n1)-c1cccc(c1)-c1ccc3c4ccccc4n(c3c1)-c1cccc-2c1] 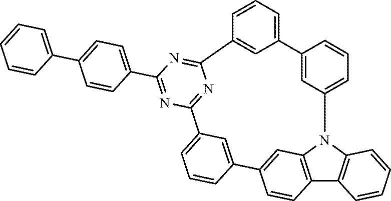 [SMILES: c1ccc(-c2ccc(-c3nc4nc(n3)-c3cccc(c3)-c3ccc5c6ccccc6n(c5c3)-c3cccc(c3)-c3cccc-4c3)cc2)cc1] 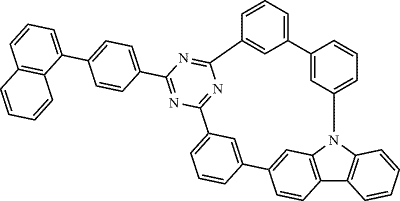 [SMILES: c1cc2cc(c1)-c1nc(-c3ccc(-c4cccc5ccccc45)cc3)nc(n1)-c1cccc(c1)-c1ccc3c4ccccc4n(c3c1)-c1cccc-2c1] 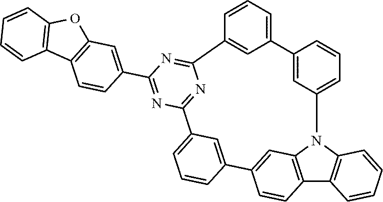 [SMILES: c1cc2cc(c1)-c1nc(nc(-c3ccc4c(c3)oc3ccccc34)n1)-c1cccc(c1)-c1ccc3c4ccccc4n(c3c1)-c1cccc-2c1] 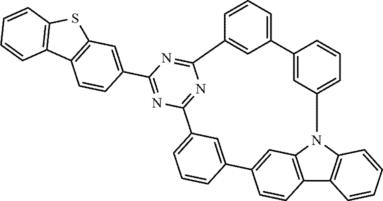 [SMILES: c1cc2cc(c1)-c1nc(nc(-c3ccc4c(c3)sc3ccccc34)n1)-c1cccc(c1)-c1ccc3c4ccccc4n(c3c1)-c1cccc-2c1] 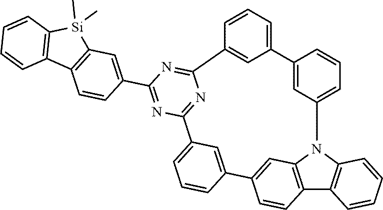 [SMILES: C[Si]1(C)c2ccccc2-c2ccc(-c3nc4nc(n3)-c3cccc(c3)-c3ccc5c6ccccc6n(c5c3)-c3cccc(c3)-c3cccc-4c3)cc21] 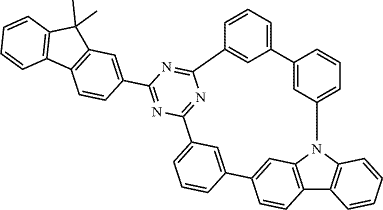 [SMILES: CC1(C)c2ccccc2-c2ccc(-c3nc4nc(n3)-c3cccc(c3)-c3ccc5c6ccccc6n(c5c3)-c3cccc(c3)-c3cccc-4c3)cc21] 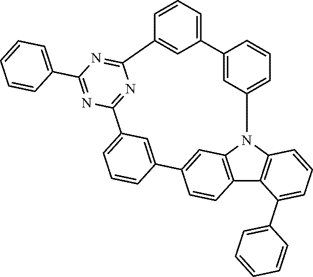 [SMILES: c1ccc(-c2nc3nc(n2)-c2cccc(c2)-c2ccc4c5c(-c6ccccc6)cccc5n(c4c2)-c2cccc(c2)-c2cccc-3c2)cc1] 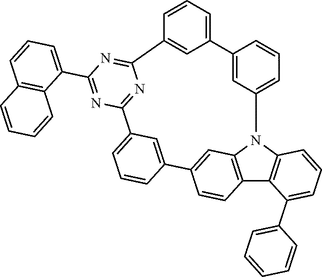 [SMILES: c1ccc(-c2cccc3c2c2ccc4cc2n3-c2cccc(c2)-c2cccc(c2)-c2nc(nc(-c3cccc5ccccc35)n2)-c2cccc-4c2)cc1] 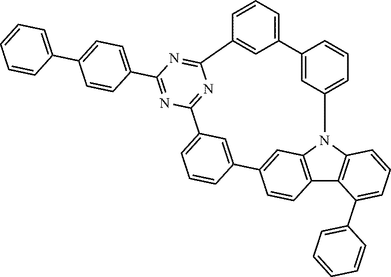 [SMILES: c1ccc(-c2ccc(-c3nc4nc(n3)-c3cccc(c3)-c3ccc5c6c(-c7ccccc7)cccc6n(c5c3)-c3cccc(c3)-c3cccc-4c3)cc2)cc1] 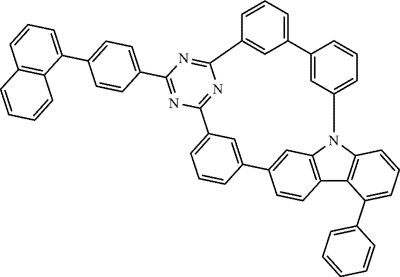 [SMILES: c1ccc(-c2cccc3c2c2ccc4cc2n3-c2cccc(c2)-c2cccc(c2)-c2nc(-c3ccc(-c5cccc6ccccc56)cc3)nc(n2)-c2cccc-4c2)cc1] 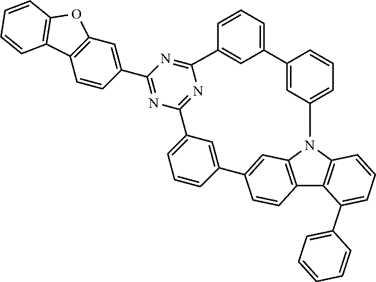 [SMILES: c1ccc(-c2cccc3c2c2ccc4cc2n3-c2cccc(c2)-c2cccc(c2)-c2nc(nc(-c3ccc5c(c3)oc3ccccc35)n2)-c2cccc-4c2)cc1] 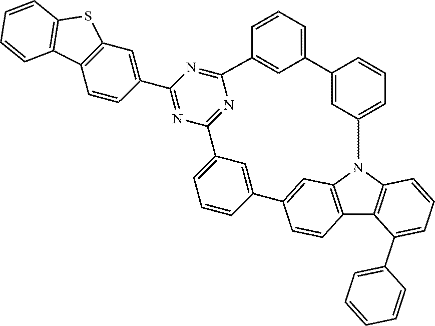 [SMILES: c1ccc(-c2cccc3c2c2ccc4cc2n3-c2cccc(c2)-c2cccc(c2)-c2nc(nc(-c3ccc5c(c3)sc3ccccc35)n2)-c2cccc-4c2)cc1] 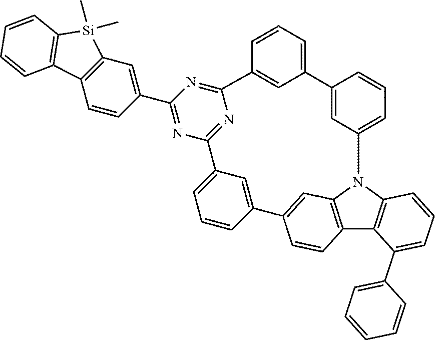 [SMILES: C[Si]1(C)c2ccccc2-c2ccc(-c3nc4nc(n3)-c3cccc(c3)-c3ccc5c6c(-c7ccccc7)cccc6n(c5c3)-c3cccc(c3)-c3cccc-4c3)cc21] 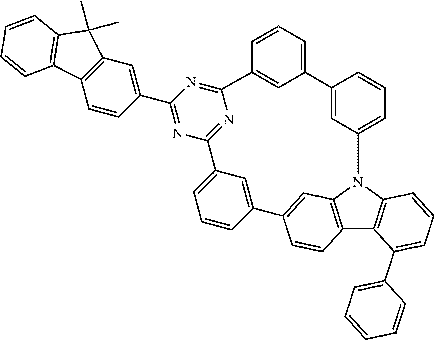 [SMILES: CC1(C)c2ccccc2-c2ccc(-c3nc4nc(n3)-c3cccc(c3)-c3ccc5c6c(-c7ccccc7)cccc6n(c5c3)-c3cccc(c3)-c3cccc-4c3)cc21] 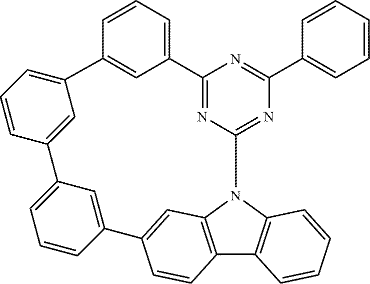 [SMILES: c1ccc(-c2nc3nc(n2)-n2c4ccccc4c4ccc(cc42)-c2cccc(c2)-c2cccc(c2)-c2cccc-3c2)cc1] 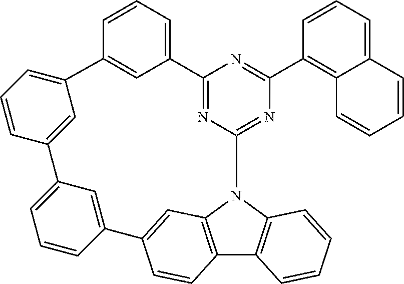 [SMILES: c1cc2cc(c1)-c1cccc(c1)-c1nc(-c3cccc4ccccc34)nc(n1)-n1c3ccccc3c3ccc(cc31)-c1cccc-2c1] 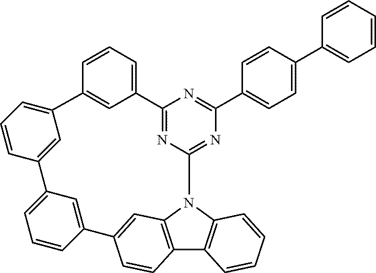 [SMILES: c1ccc(-c2ccc(-c3nc4nc(n3)-n3c5ccccc5c5ccc(cc53)-c3cccc(c3)-c3cccc(c3)-c3cccc-4c3)cc2)cc1] 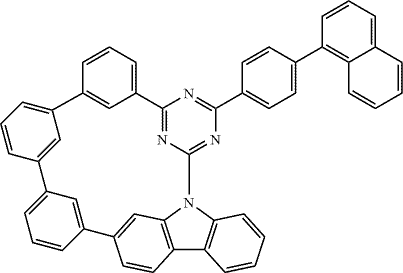 [SMILES: c1cc2cc(c1)-c1cccc(c1)-c1nc(-c3ccc(-c4cccc5ccccc45)cc3)nc(n1)-n1c3ccccc3c3ccc(cc31)-c1cccc-2c1] 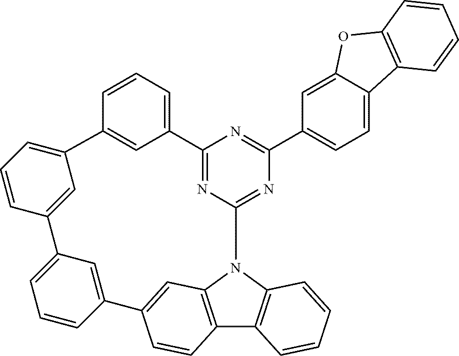 [SMILES: c1cc2cc(c1)-c1cccc(c1)-c1nc(-c3ccc4c(c3)oc3ccccc34)nc(n1)-n1c3ccccc3c3ccc(cc31)-c1cccc-2c1] 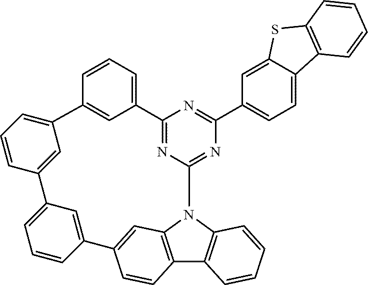 [SMILES: c1cc2cc(c1)-c1cccc(c1)-c1nc(-c3ccc4c(c3)sc3ccccc34)nc(n1)-n1c3ccccc3c3ccc(cc31)-c1cccc-2c1] 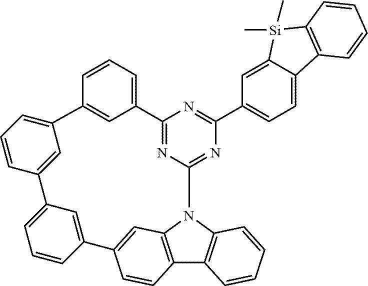 [SMILES: C[Si]1(C)c2ccccc2-c2ccc(-c3nc4nc(n3)-n3c5ccccc5c5ccc(cc53)-c3cccc(c3)-c3cccc(c3)-c3cccc-4c3)cc21] 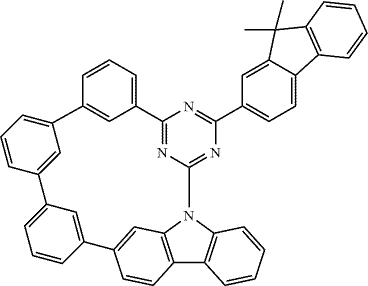 [SMILES: CC1(C)c2ccccc2-c2ccc(-c3nc4nc(n3)-n3c5ccccc5c5ccc(cc53)-c3cccc(c3)-c3cccc(c3)-c3cccc-4c3)cc21] 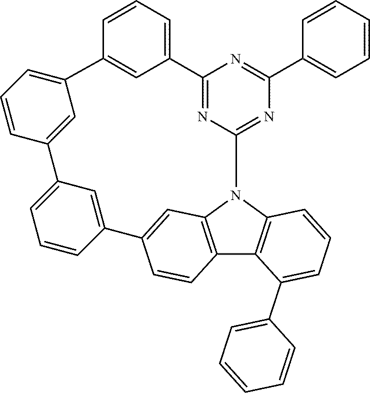 [SMILES: c1ccc(-c2nc3nc(n2)-n2c4cc(ccc4c4c(-c5ccccc5)cccc42)-c2cccc(c2)-c2cccc(c2)-c2cccc-3c2)cc1] 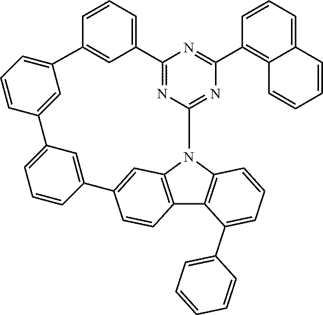 [SMILES: c1ccc(-c2cccc3c2c2ccc4cc2n3-c2nc(nc(-c3cccc5ccccc35)n2)-c2cccc(c2)-c2cccc(c2)-c2cccc-4c2)cc1] 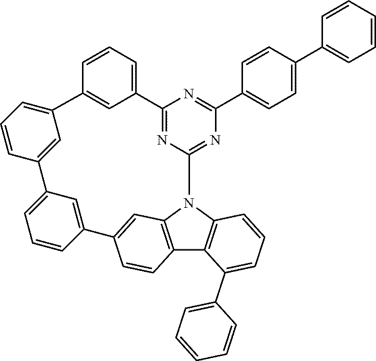 [SMILES: c1ccc(-c2ccc(-c3nc4nc(n3)-n3c5cc(ccc5c5c(-c6ccccc6)cccc53)-c3cccc(c3)-c3cccc(c3)-c3cccc-4c3)cc2)cc1] 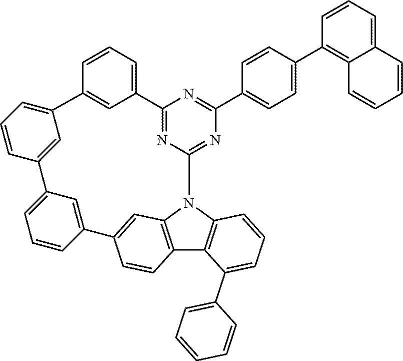 [SMILES: c1ccc(-c2cccc3c2c2ccc4cc2n3-c2nc(-c3ccc(-c5cccc6ccccc56)cc3)nc(n2)-c2cccc(c2)-c2cccc(c2)-c2cccc-4c2)cc1] 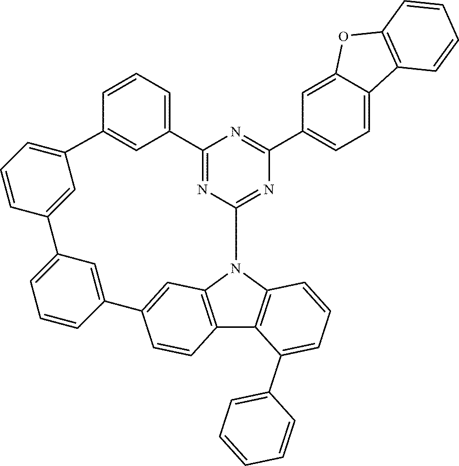 [SMILES: c1ccc(-c2cccc3c2c2ccc4cc2n3-c2nc(nc(-c3ccc5c(c3)oc3ccccc35)n2)-c2cccc(c2)-c2cccc(c2)-c2cccc-4c2)cc1] 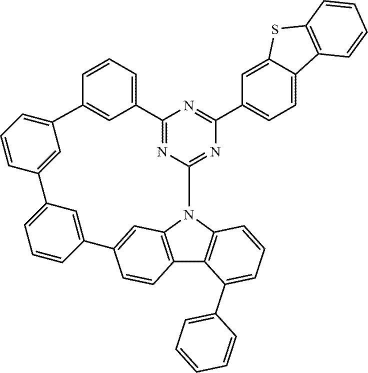 [SMILES: c1ccc(-c2cccc3c2c2ccc4cc2n3-c2nc(nc(-c3ccc5c(c3)sc3ccccc35)n2)-c2cccc(c2)-c2cccc(c2)-c2cccc-4c2)cc1] 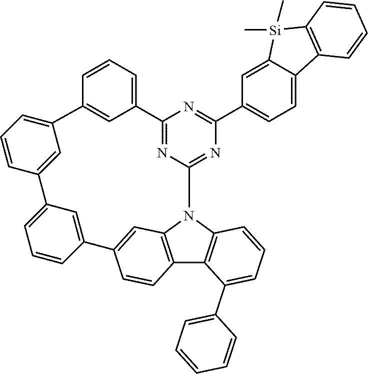 [SMILES: C[Si]1(C)c2ccccc2-c2ccc(-c3nc4nc(n3)-n3c5cc(ccc5c5c(-c6ccccc6)cccc53)-c3cccc(c3)-c3cccc(c3)-c3cccc-4c3)cc21] 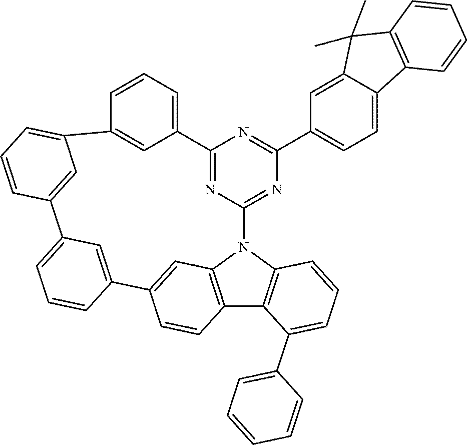 [SMILES: CC1(C)c2ccccc2-c2ccc(-c3nc4nc(n3)-n3c5cc(ccc5c5c(-c6ccccc6)cccc53)-c3cccc(c3)-c3cccc(c3)-c3cccc-4c3)cc21] 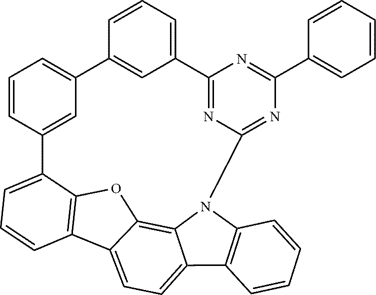 [SMILES: c1ccc(-c2nc3nc(n2)n2c4ccccc4c4ccc5c6cccc(c7cccc(c7)c7cccc3c7)c6oc5c42)cc1] 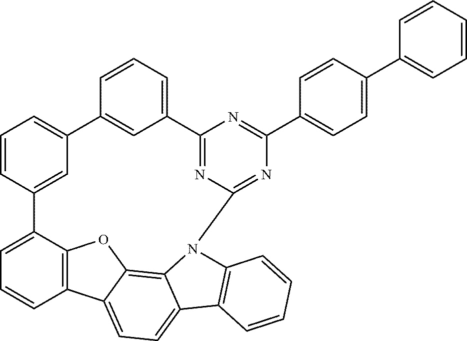 [SMILES: c1ccc(-c2ccc(-c3nc4nc(n3)n3c5ccccc5c5ccc6c7cccc(c8cccc(c8)c8cccc4c8)c7oc6c53)cc2)cc1] 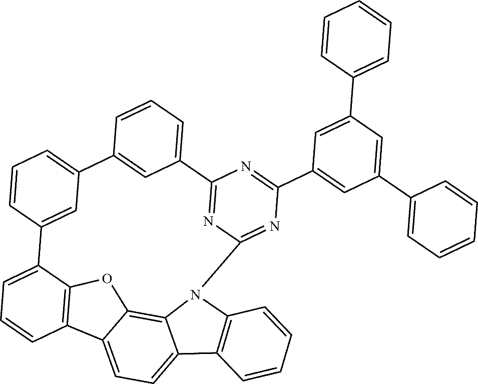 [SMILES: c1ccc(-c2cc(-c3ccccc3)cc(-c3nc4nc(n3)n3c5ccccc5c5ccc6c7cccc(c8cccc(c8)c8cccc4c8)c7oc6c53)c2)cc1] 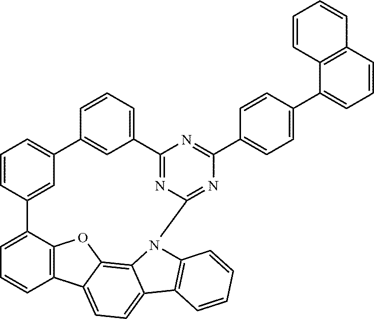 [SMILES: c1cc2cc(c1)c1nc(-c3ccc(-c4cccc5ccccc45)cc3)nc(n1)n1c3ccccc3c3ccc4c5cccc(c6cccc2c6)c5oc4c31] 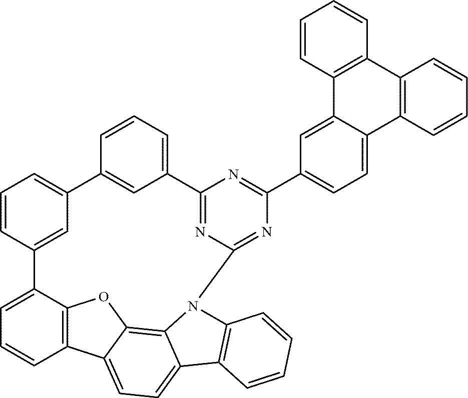 [SMILES: c1cc2cc(c1)c1nc(-c3ccc4c5ccccc5c5ccccc5c4c3)nc(n1)n1c3ccccc3c3ccc4c5cccc(c6cccc2c6)c5oc4c31] 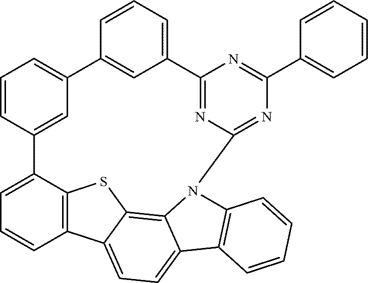 [SMILES: c1ccc(-c2nc3nc(n2)n2c4ccccc4c4ccc5c6cccc(c7cccc(c7)c7cccc3c7)c6sc5c42)cc1] 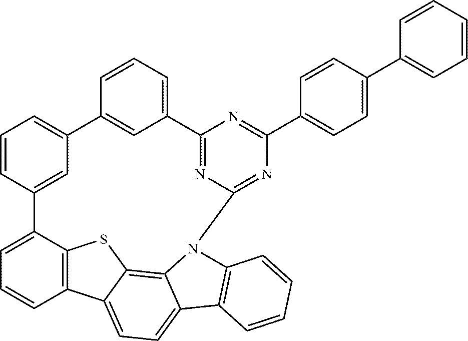 [SMILES: c1ccc(-c2ccc(-c3nc4nc(n3)n3c5ccccc5c5ccc6c7cccc(c8cccc(c8)c8cccc4c8)c7sc6c53)cc2)cc1] 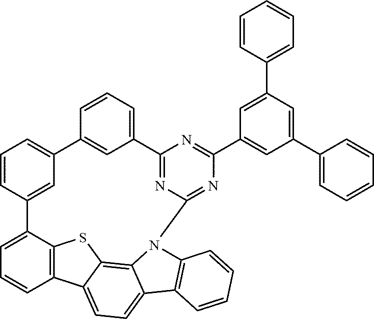 [SMILES: c1ccc(-c2cc(-c3ccccc3)cc(-c3nc4nc(n3)n3c5ccccc5c5ccc6c7cccc(c8cccc(c8)c8cccc4c8)c7sc6c53)c2)cc1] 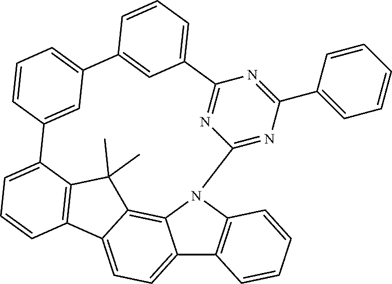 [SMILES: CC1(C)c2c3cccc2-c2ccc4c5ccccc5n(c4c21)-c1nc(-c2ccccc2)nc(n1)-c1cccc(c1)-c1cccc-3c1] 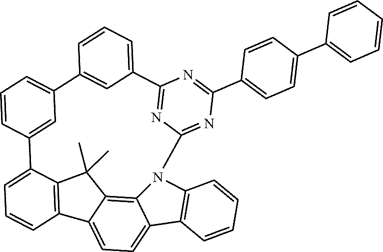 [SMILES: CC1(C)c2c3cccc2-c2ccc4c5ccccc5n(c4c21)-c1nc(-c2ccc(-c4ccccc4)cc2)nc(n1)-c1cccc(c1)-c1cccc-3c1] 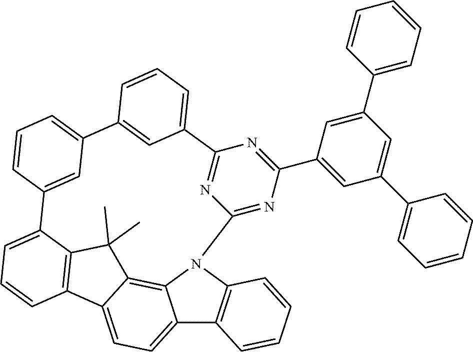 [SMILES: CC1(C)c2c3cccc2-c2ccc4c5ccccc5n(c4c21)-c1nc(nc(-c2cc(-c4ccccc4)cc(-c4ccccc4)c2)n1)-c1cccc(c1)-c1cccc-3c1] 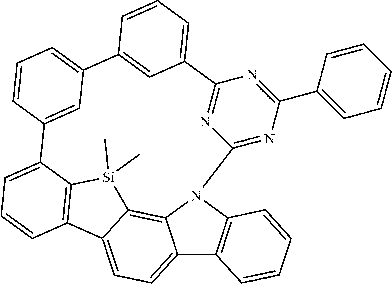 [SMILES: C[Si]1(C)c2c3cccc2-c2ccc4c5ccccc5n(c4c21)-c1nc(-c2ccccc2)nc(n1)-c1cccc(c1)-c1cccc-3c1] 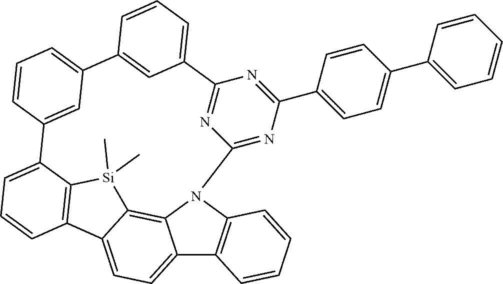 [SMILES: C[Si]1(C)c2c3cccc2-c2ccc4c5ccccc5n(c4c21)-c1nc(-c2ccc(-c4ccccc4)cc2)nc(n1)-c1cccc(c1)-c1cccc-3c1] 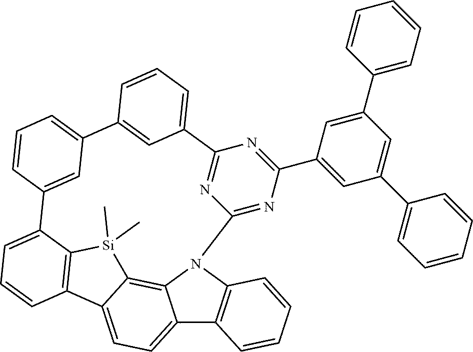 [SMILES: C[Si]1(C)c2c3cccc2-c2ccc4c5ccccc5n(c4c21)-c1nc(nc(-c2cc(-c4ccccc4)cc(-c4ccccc4)c2)n1)-c1cccc(c1)-c1cccc-3c1] 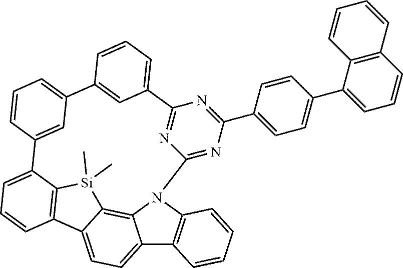 [SMILES: C[Si]1(C)c2c3cccc2-c2ccc4c5ccccc5n(c4c21)-c1nc(-c2ccc(-c4cccc5ccccc45)cc2)nc(n1)-c1cccc(c1)-c1cccc-3c1] 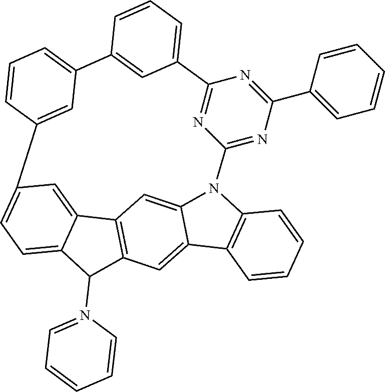 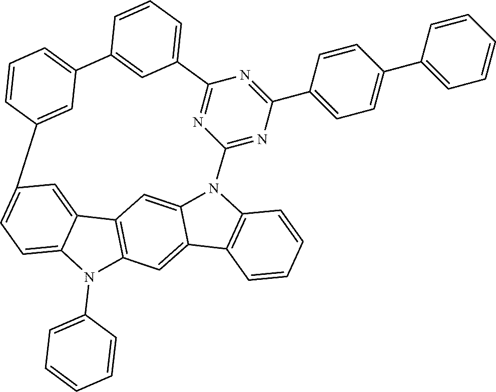 [SMILES: c1ccc(-c2ccc(-c3nc4nc(n3)n3c5ccccc5c5cc6c(cc53)c3cc(ccc3n6-c3ccccc3)c3cccc(c3)c3cccc4c3)cc2)cc1] 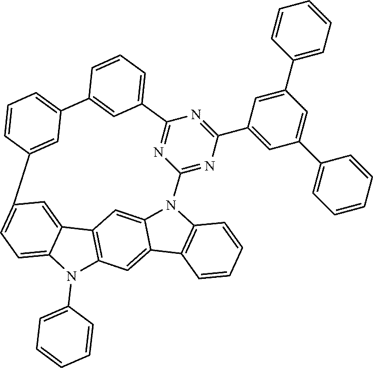 [SMILES: c1ccc(-c2cc(-c3ccccc3)cc(-c3nc4nc(n3)n3c5ccccc5c5cc6c(cc53)c3cc(ccc3n6-c3ccccc3)c3cccc(c3)c3cccc4c3)c2)cc1] 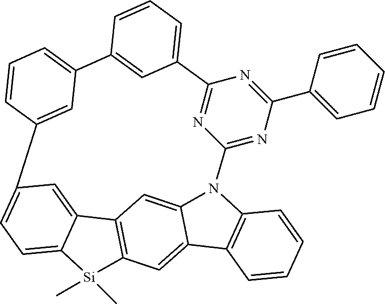 [SMILES: C[Si]1(C)c2ccc3cc2-c2cc4c(cc21)c1ccccc1n4-c1nc(-c2ccccc2)nc(n1)-c1cccc(c1)-c1cccc-3c1] 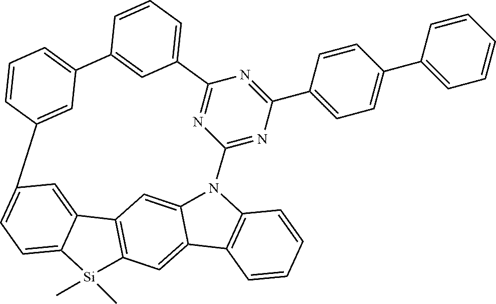 [SMILES: C[Si]1(C)c2ccc3cc2-c2cc4c(cc21)c1ccccc1n4-c1nc(-c2ccc(-c4ccccc4)cc2)nc(n1)-c1cccc(c1)-c1cccc-3c1] 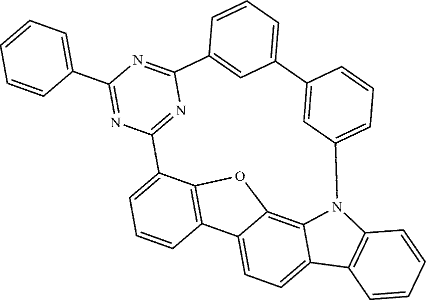 [SMILES: c1ccc(-c2nc3nc(n2)c2cccc4c5ccc6c7ccccc7n(c7cccc(c7)c7cccc3c7)c6c5oc24)cc1] 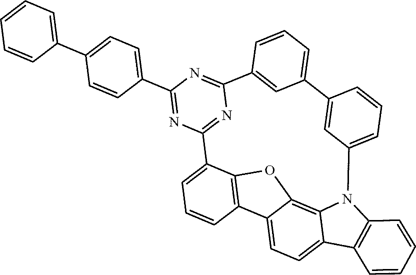 [SMILES: c1ccc(-c2ccc(-c3nc4nc(n3)c3cccc5c6ccc7c8ccccc8n(c8cccc(c8)c8cccc4c8)c7c6oc35)cc2)cc1] 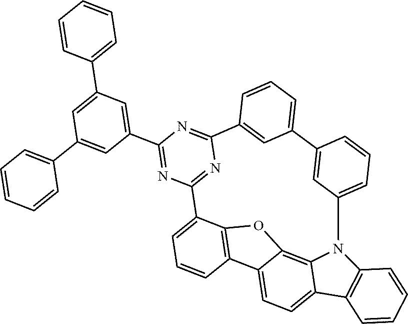 [SMILES: c1ccc(-c2cc(-c3ccccc3)cc(-c3nc4nc(n3)c3cccc5c6ccc7c8ccccc8n(c8cccc(c8)c8cccc4c8)c7c6oc35)c2)cc1] 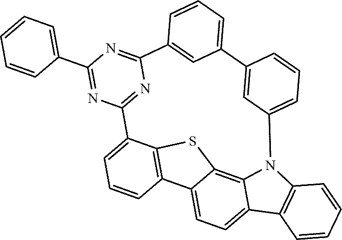 [SMILES: c1ccc(-c2nc3nc(n2)c2cccc4c5ccc6c7ccccc7n(c7cccc(c7)c7cccc3c7)c6c5sc24)cc1] 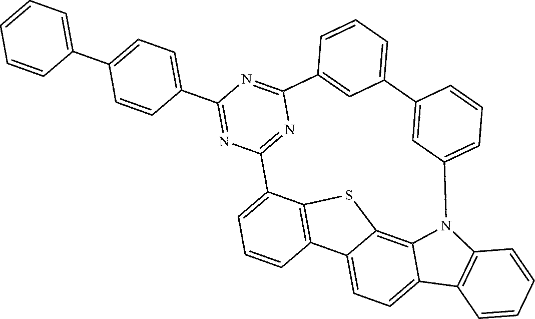 [SMILES: c1ccc(-c2ccc(-c3nc4nc(n3)c3cccc5c6ccc7c8ccccc8n(c8cccc(c8)c8cccc4c8)c7c6sc35)cc2)cc1] 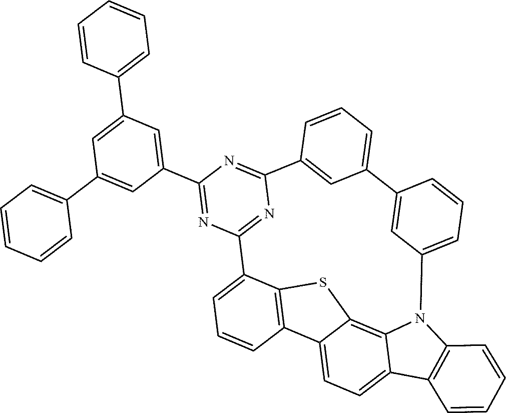 [SMILES: c1ccc(-c2cc(-c3ccccc3)cc(-c3nc4nc(n3)c3cccc5c6ccc7c8ccccc8n(c8cccc(c8)c8cccc4c8)c7c6sc35)c2)cc1] 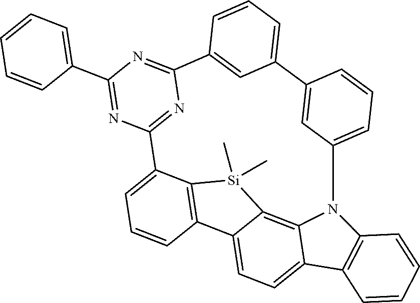 [SMILES: C[Si]1(C)c2c3cccc2-c2ccc4c5ccccc5n(c4c21)-c1cccc(c1)-c1cccc(c1)-c1nc(-c2ccccc2)nc-3n1] 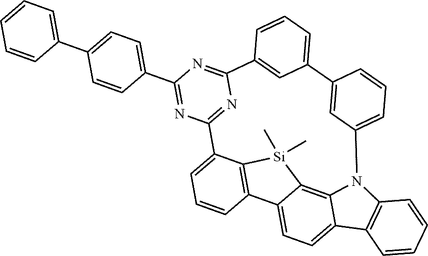 [SMILES: C[Si]1(C)c2c3cccc2-c2ccc4c5ccccc5n(c4c21)-c1cccc(c1)-c1cccc(c1)-c1nc(-c2ccc(-c4ccccc4)cc2)nc-3n1] 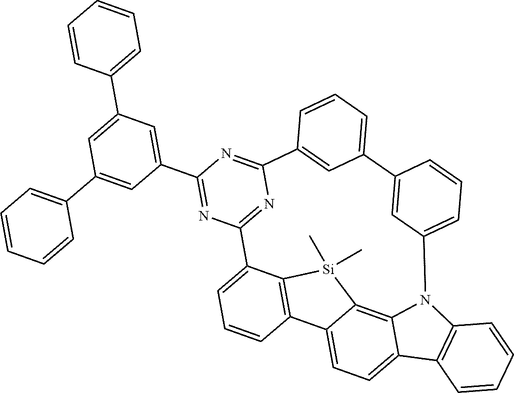 [SMILES: C[Si]1(C)c2c3cccc2-c2ccc4c5ccccc5n(c4c21)-c1cccc(c1)-c1cccc(c1)-c1nc(-c2cc(-c4ccccc4)cc(-c4ccccc4)c2)nc-3n1] 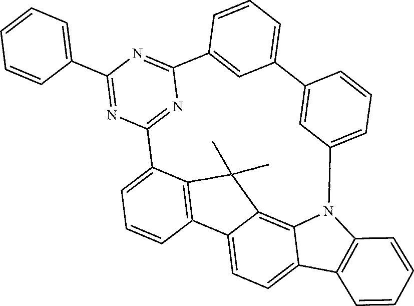 [SMILES: CC1(C)c2c3cccc2-c2ccc4c5ccccc5n(c4c21)-c1cccc(c1)-c1cccc(c1)-c1nc(-c2ccccc2)nc-3n1] 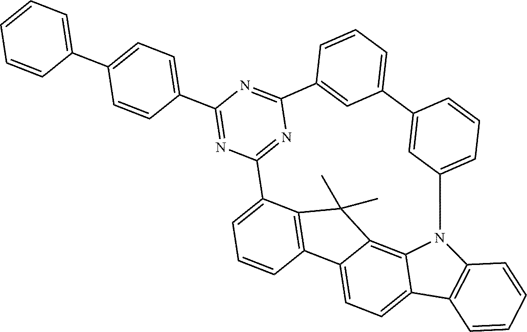 [SMILES: CC1(C)c2c3cccc2-c2ccc4c5ccccc5n(c4c21)-c1cccc(c1)-c1cccc(c1)-c1nc(-c2ccc(-c4ccccc4)cc2)nc-3n1] 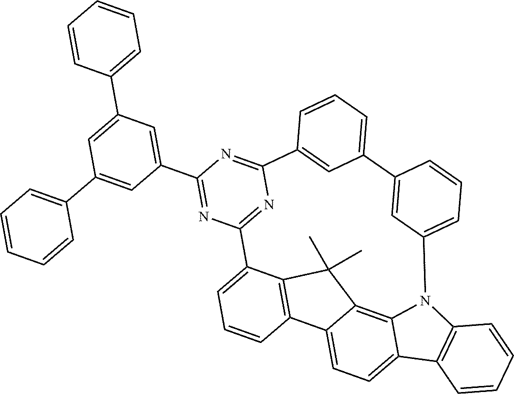 [SMILES: CC1(C)c2c3cccc2-c2ccc4c5ccccc5n(c4c21)-c1cccc(c1)-c1cccc(c1)-c1nc(-c2cc(-c4ccccc4)cc(-c4ccccc4)c2)nc-3n1] 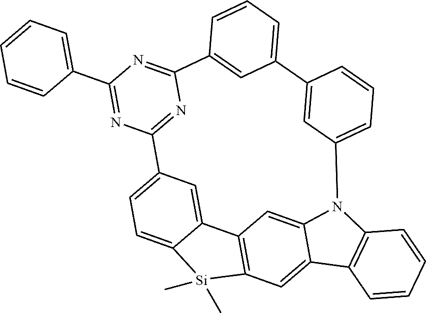 [SMILES: C[Si]1(C)c2ccc3cc2-c2cc4c(cc21)c1ccccc1n4-c1cccc(c1)-c1cccc(c1)-c1nc(-c2ccccc2)nc-3n1] 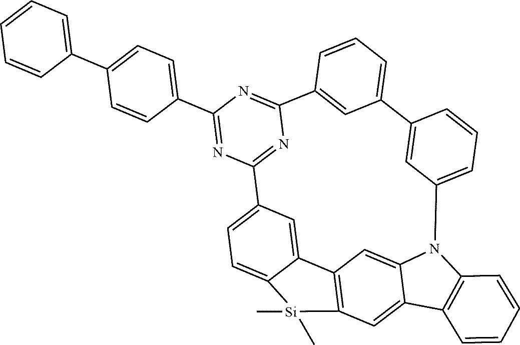 [SMILES: C[Si]1(C)c2ccc3cc2-c2cc4c(cc21)c1ccccc1n4-c1cccc(c1)-c1cccc(c1)-c1nc(-c2ccc(-c4ccccc4)cc2)nc-3n1] 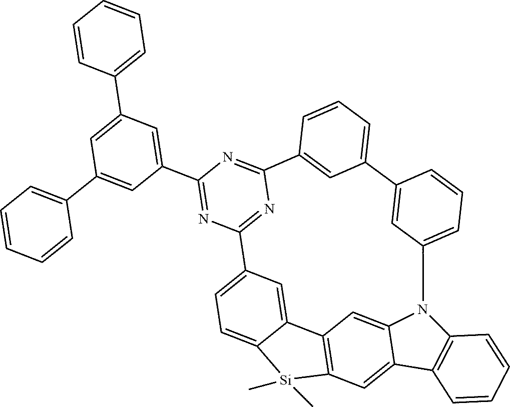 [SMILES: C[Si]1(C)c2ccc3cc2-c2cc4c(cc21)c1ccccc1n4-c1cccc(c1)-c1cccc(c1)-c1nc(-c2cc(-c4ccccc4)cc(-c4ccccc4)c2)nc-3n1] 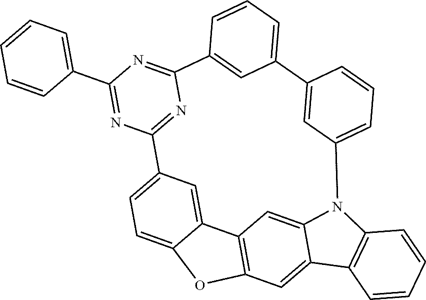 [SMILES: c1ccc(-c2nc3nc(n2)c2ccc4oc5cc6c7ccccc7n(c7cccc(c7)c7cccc3c7)c6cc5c4c2)cc1] 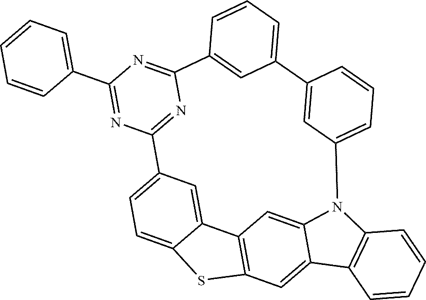 [SMILES: c1ccc(-c2nc3nc(n2)c2ccc4sc5cc6c7ccccc7n(c7cccc(c7)c7cccc3c7)c6cc5c4c2)cc1] 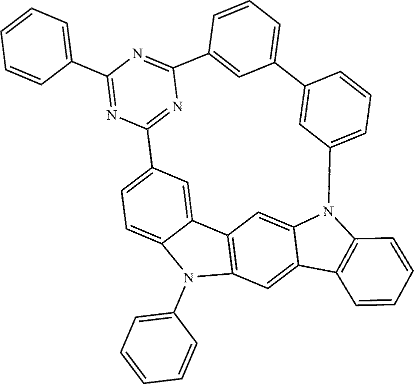 [SMILES: c1ccc(-c2nc3nc(n2)c2ccc4c(c2)c2cc5c(cc2n4-c2ccccc2)c2ccccc2n5c2cccc(c2)c2cccc3c2)cc1] 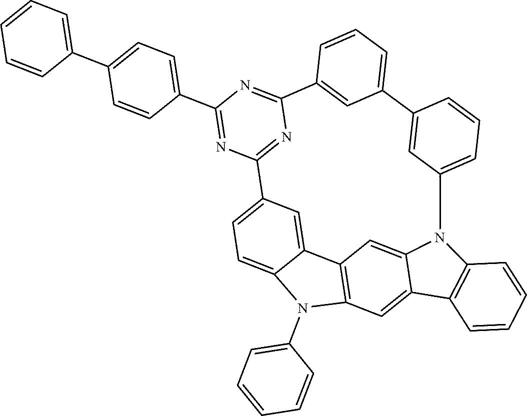 [SMILES: c1ccc(-c2ccc(-c3nc4nc(n3)c3ccc5c(c3)c3cc6c(cc3n5-c3ccccc3)c3ccccc3n6c3cccc(c3)c3cccc4c3)cc2)cc1] 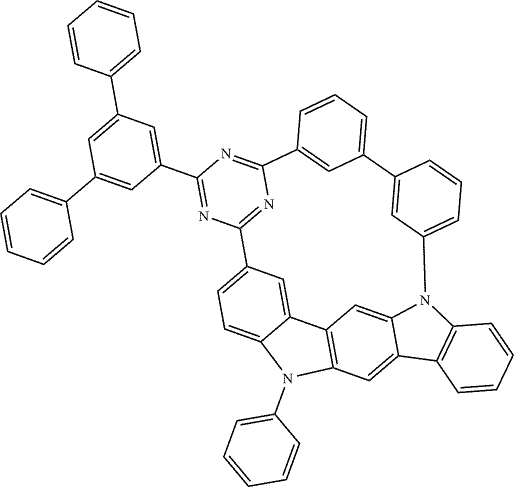 [SMILES: c1ccc(-c2cc(-c3ccccc3)cc(-c3nc4nc(n3)c3ccc5c(c3)c3cc6c(cc3n5-c3ccccc3)c3ccccc3n6c3cccc(c3)c3cccc4c3)c2)cc1] 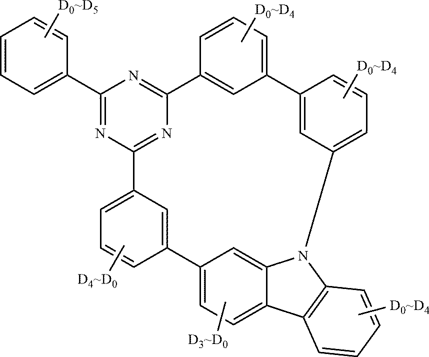 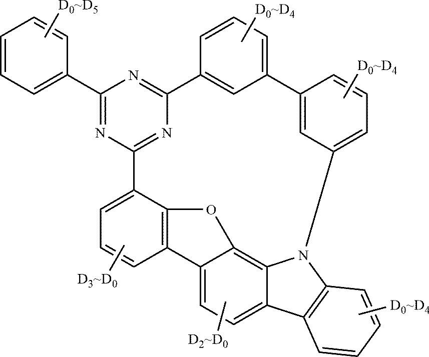 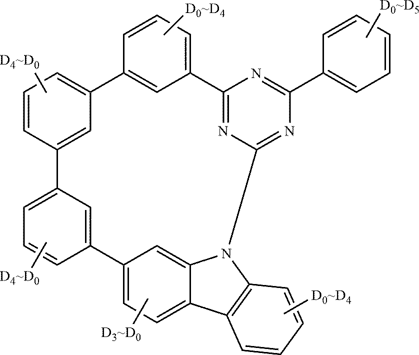 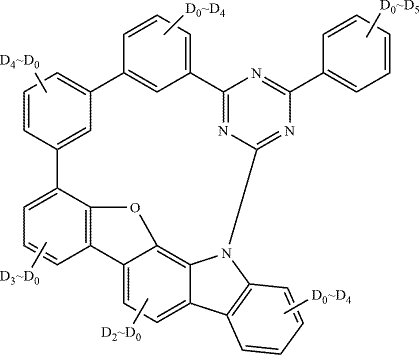 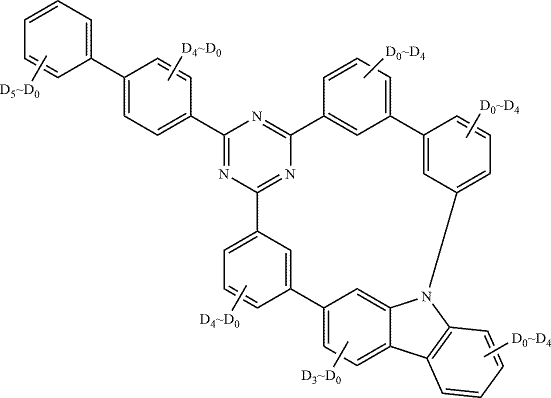 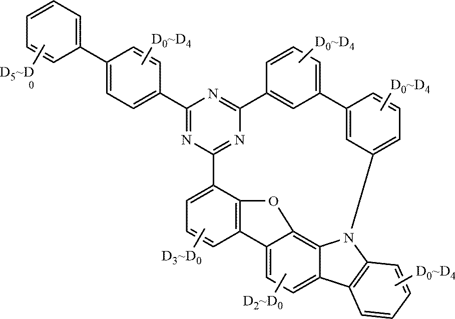 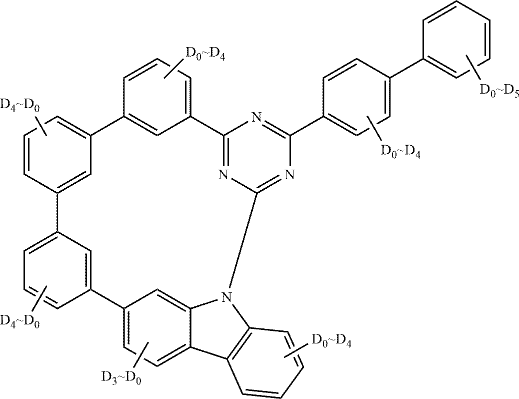 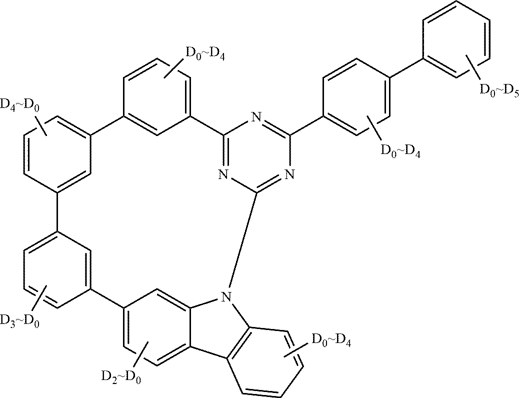
.

10. A composition for an organic optoelectronic device, comprising: a first compound and a second compound,
wherein the first compound is the compound as claimed in claim 1, and
the second compound is a compound represented by Chemical Formula 2, or a compound represented by a combination of Chemical Formula 3 and Chemical Formula 4: 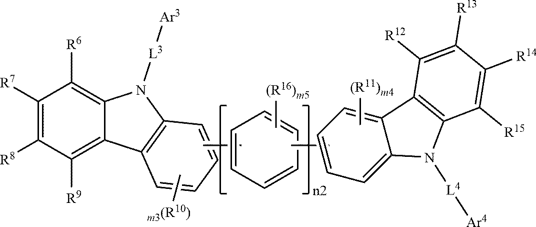
wherein, in Chemical Formula 2,
Ar³ and Ar⁴ are each independently a substituted or unsubstituted C6 to C20 aryl group or a substituted or unsubstituted C2 to C30 heterocyclic group,
L³ and L⁴ are each independently a single bond or a substituted or unsubstituted C6 to C20 arylene group,
R⁶ to R¹⁶ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
m3 and m4 are each independently an integer from 1 to 3,
m5 is an integer from 1 to 4, and
n2 is an integer from 0 to 2; 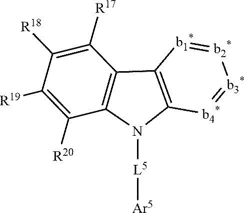 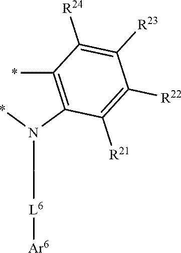
wherein, in Chemical Formulas 3 and 4,
Ar⁵ and Ar⁶ are each independently a substituted or unsubstituted C6 to C20 aryl group or a substituted or unsubstituted C2 to C30 heterocyclic group,
adjacent two of b₁^{∗} to b₄^{∗} of Chemical Formula 3 are each a carbon (C) linked to * in Chemical Formula 4,
the other two not linked to ^{∗} of Chemical Formula 4 of b₁^{∗}to b₄^{∗} in Chemical Formula 3 are each independently C- L^{d}-R^{f},
L^{d}, L⁵, and L⁶ are each independently a single bond or a substituted or unsubstituted C6 to C20 arylene group, and
R^{f} and R¹⁷ to R²⁴ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group.

11. The composition as claimed in claim 10, wherein the second compound is the compound represented by Chemical Formula 2, and Chemical Formula 2 is represented by Chemical Formula 2-8: 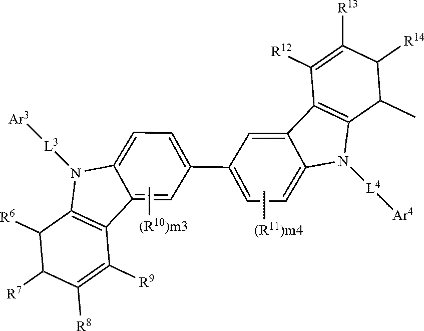
wherein, in Chemical Formula 2–8,
R⁶ to R¹⁵ are each independently hydrogen, deuterium, or a substituted or unsubstituted C6 to C12 aryl group,
m3 and m4 are each independently an integer from 1 to 3, and
^{∗}-L³-Ar³ and *-L⁴-Ar⁴ are each independently one of the substituents of Group II, 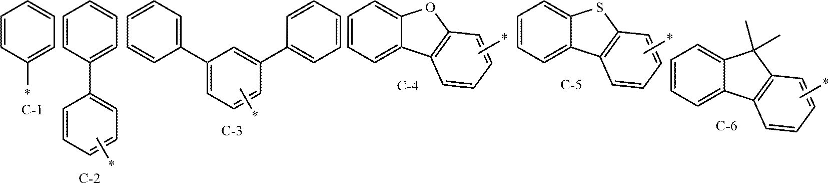 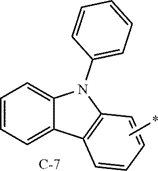 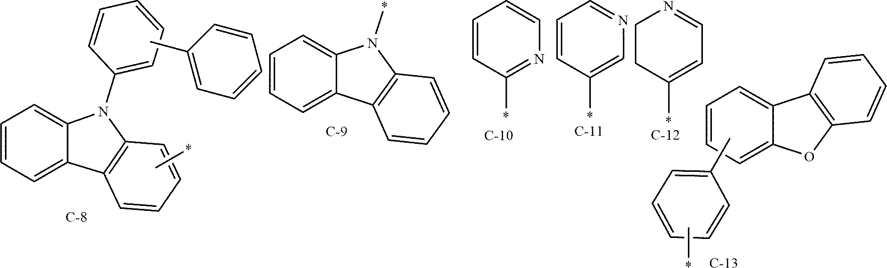 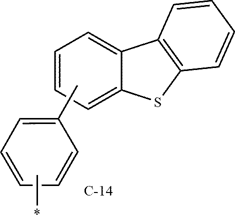 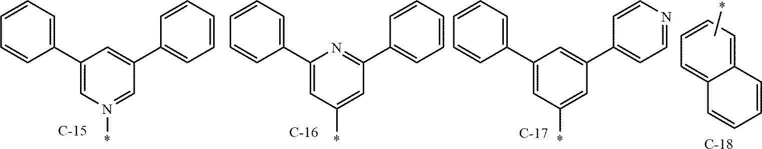 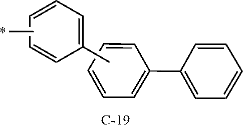 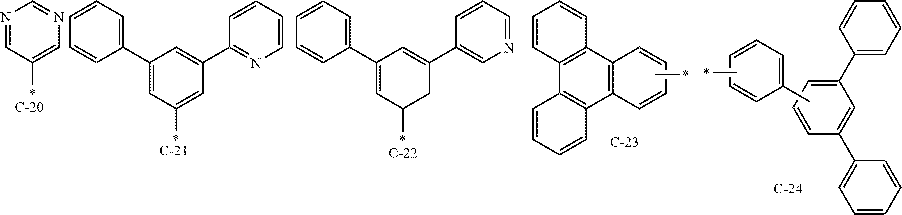 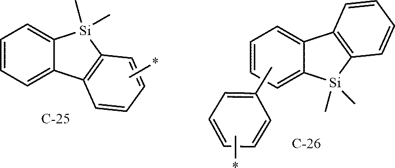
wherein, in Group II, * is a linking point.

12. The compound as claimed in claim 10, wherein the second compound is the compound represented by the combination of Chemical Formula 3 and Chemical Formula 4, and the combination of Chemical Formula 3 and Chemical Formula 4 is represented by Chemical Formula 3C: 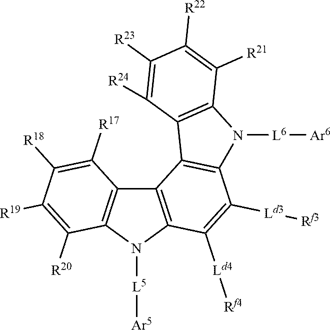
wherein, in Chemical Formula 3C,
L^{d3} and L^{d4} are each a single bond,
L⁵ and L⁶ are each independently a single bond or a substituted or unsubstituted C6 to C12 arylene group,
R¹⁷ to R²⁴, R^{f3}, and R^{f4} are each independently hydrogen, deuterium, or a C6 to C12 aryl group, and
Ar⁵ and Ar⁶ are each independently a substituted or unsubstituted phenyl group, a substituted or unsubstituted naphthyl group, or a substituted or unsubstituted biphenyl group.

13. An organic optoelectronic device, comprising:
an anode and a cathode facing each other, and
at least one organic layer between the anode and the cathode,
wherein the at least one organic layer includes the compound as claimed in claim 1.

14. The organic optoelectronic device as claimed in claim 13, wherein:
the at least one organic layer includes a light emitting layer, and
the light emitting layer includes the compound for the organic optoelectronic device.

15. A display device comprising the organic optoelectronic device as claimed in claim 13.

16. An organic optoelectronic device, comprising:
an anode and a cathode facing each other, and
at least one organic layer between the anode and the cathode,
wherein the at least one organic layer includes the composition for the organic optoelectronic device as claimed in claim 10.

17. The organic optoelectronic device as claimed in claim 16, wherein:
the at least one organic layer includes a light emitting layer, and
the light emitting layer includes the composition for the organic optoelectronic device.

18. A display device comprising the organic optoelectronic device as claimed in claim 16.
